(19) United States
(12) Patent Application Publication
(10) Pub. No.: US 2025/0171778 A1
(43) Pub. Date: May 29, 2025

(54) Treatments of Disorders of Myelin

(71) Applicant: Myrtelle Inc., Wakefield, MA (US)

(72) Inventors: Richard T. Layer, Marlborough, MA (US); Mark Pykett, York Harbor, ME (US); Jeffrey Allen, Londonberry, NH (US)

(73) Assignee: Myrtelle Inc., Wakefield, MA (US)

(21) Appl. No.: 18/859,602

(22) PCT Filed: Apr. 24, 2023
(86) PCT No.: PCT/US2023/066117

§ 371 Date: Oct. 24, 2024

(30) Foreign Application Priority Data
Apr. 25, 2022 (US) 63363529

(51) Int. Cl. C12N 15/113; A61P 25/00; C12N 15/11; C12N 15/86

(52) U.S. Cl. CPC C12N 15/113; A61P 25/00; C12N 15/111; C12N 15/86; C12N 2310/141; C12N 2750/14143; C12N 2750/14145; C12N 2830/50

(57) ABSTRACT

The present disclosure relates to RNA molecules, recombinant nucleic acids, and gene therapy vectors for use in the treatment of diseases and disorders associated with a deficiency or dysfunction of myelin.

### CROSS REFERENCE TO RELATED APPLICATION

This application claims priority to U.S. Provisional Application No. 63/363,529 filed on Apr. 25, 2022. The content of the application is incorporated herein by reference in its entirety.

### REFERENCE TO A SEQUENCE LISTING

This application incorporates by reference the Sequence Listing submitted in Computer Readable Form as file Sequence_Listing_323429-00201.xml, created on Apr. 19, 2023 and containing 258,210 bytes.

### FIELD OF THE INVENTION

This disclosure relates to compositions and methods for treating inherited and/or acquired disorders of myelin.

### BACKGROUND

The human central nervous system (CNS), including the brain and spinal cord, is composed of two types of cells, neurons and glia. Neurons have a cell body that holds the nucleus (where the cell's genes are located), an axon that extends away from the cell body (ending in an axon terminal), and dendrites that branch off from the cell body and make connections with axon terminals from other neurons. The neuron receives information at the cell body and dendrites (from other neurons) and sends this information to other neurons along its axon. Information travels down the length of the axon in the form of an electrical signal known as an action potential. At the end of the axon, the action potential triggers the release of neurotransmitters (into the synapse, a small space between the cells across which the neurotransmitters move). All the activities of the central nervous system, i.e., thinking, processing sensory information, storing memories, and controlling muscles and glands, are performed by interconnected networks of neurons.

The glial cells of the CNS support the function of neurons. These cells include astrocytes, microglia, and oligodendrocytes. Astrocytes comprise the blood-brain barrier (BBB), provide nutrients to neurons, and maintain extracellular ion balance and neurotransmitter levels. Microglia are phagocytic cells similar to macrophages that migrate through the CNS and remove damaged or unnecessary material. Oligodendrocytes are the myelin-producing cells of the central nervous system.

Myelin is a structurally complex substance composed of high levels of saturated, long-chain fatty acids, glycosphingolipids, cholesterol, and proteins. Oligodendrocytes create myelin by extending sheet like processes, each of which contacts and wraps tightly around a segment of axon multiple times to create the unique architecture of the myelin sheath. On the same axon, adjacent myelin segments belong to different oligodendrocytes, and a single oligodendrocyte can myelinate up to 50 axonal segments (Stadelmann et al., 2019). Oligodendrocyte myelin facilitates rapid conduction of action potentials and supports axonal metabolic needs. Oligodendrocytes also participate in extracellular fluid regulation and provide neuronal trophic support through production of neurotrophic factors such as glial cell line-derived neurotrophic factor (GDNF), brain-derived neurotrophic factor (BDNF), or insulin-like growth factor-1 (IGF-1) (Bradl and Lassmann, 2010).

The central nervous system is organized into “gray matter,” which generally contains the cell bodies and dendrite networks of neurons, and “white matter,” which consists of axon bundles encased by myelin produced by oligodendrocytes. The myelin sheath has a high lipid fat content, which accounts for the whitish appearance. Myelin plays a critical role in neuronal communication. Impairment of oligodendrocytes disrupts white matter integrity and results in white matter degeneration (demyelination) and loss of neuronal communication within the brain and spinal cord.

Myelin-related disorders, inherited or acquired, impact millions of people, levying a heavy burden on affected individuals and their families. The pathological processes underlying many of these disorders remain poorly understood and few disease-modifying therapies exist. Thus, there is need for therapeutics for treating these disorders.

### SUMMARY

This disclosure addresses the need mentioned above in a number of aspects.

One aspect of the invention provides a method of reducing expression of a gene in an oligodendrocyte or a method of treating an inherited or acquired disorder of myelin, the method comprising providing a composition which comprises an adeno-associated virus (AAV) particle with preferential tropism for the oligodendrocyte cell surface; wherein the AAV particle encapsidates a polynucleotide; wherein the polynucleotide comprises a 5′ inverted terminal repeat (ITR), a promoter sequence region, a polynucleotide encoding a pri- or pre-miRNA targeting said gene, an optional post-transcriptional regulatory element (e.g., a woodchuck hepatitis post-transcriptional regulatory element), a polyA signal sequence region, and a 3′ ITR.

In certain embodiments, the polynucleotide is a pri- or pre-miRNA scaffold derived from human mir-16-1, miR-21, miR-23a, miRNA-30a, miR-31, miR-122, miR-155, or miR-451, wherein the pri- or pre-miRNA scaffold excludes the native sequence for the guide strand and the passenger strand of the pre-miRNA; wherein a heterologous guide strand is inserted into the scaffold as a replacement for the native sequence of the guide strand, wherein the heterologous guide strand is complementarity to the mRNA of the target gene, wherein upon processing of the pri- or pre-miRNA by a cytosolic nuclease the heterologous guide strand is incorporated into a RISC complex to permit the RISC complex to target the mRNA of the target gene and down regulate expression of the gene.

In certain embodiments, the heterologous guide strand is complementarity to the mRNA of a protein the elimination of which improves a treatment outcome of an inherited or acquired disorder of myelin, such as Pelizaeus-Merzbacher disease, multiple system atrophy, or Hypomyelination with Atrophy of the Basal Ganglia and Cerebellum.

In one aspect, the disclosure provides a method of reducing expression of a target gene in an oligodendrocyte or a method of treating an inherited or acquired disorder of myelin. The method comprises (1) providing an AAV particle with preferential tropism for the oligodendrocyte cell surface; wherein the AAV particle encapsidates a nucleic acid that comprises from 5′ to 3′: a 5′ ITR, a promoter sequence region, a polynucleotide encoding a pri- or pre-miRNA targeting said target gene, and a 3′ ITR, and (2) contacting the AAV particle with the oligodendrocyte. In some embodiments, the nucleic acid can further comprise one or more of a post-transcriptional regulatory element and a polyA signal sequence region between the polynucleotide and the 3′ ITR. The pri- or pre-miRNA may comprise (a) a pri- or pre-miRNA scaffold, (b) a heterologous guide strand, and (c) a heterologous passenger strand.

In one embodiment, the pri- or pre-miRNA scaffold is a human pri- or pre-miRNA scaffold derived from a human microRNA. Examples of the human microRNA include those derived from human mir-16-1, miR-21, miR-23a, miRNA-30a, miR-31, miR-122, miR-155, or miR-451. Preferably, the human pri- or pre-miRNA scaffold does not include the native sequence for the native guide strand and the native sequence for the native passenger strand of the human microRNA. The heterologous guide strand can be inserted into the human pri- or pre-miRNA scaffold as a replacement for the native sequence of the native guide strand. The heterologous guide strand may be complementarity to the mRNA of the target gene. Upon processing of the pri- or pre-miRNA by a cytosolic nuclease the heterologous guide strand is incorporated into a RISC complex to permit the RISC complex to target the mRNA of the target gene and down regulate expression of the target gene.

In the method described above, the heterologous guide strand can be complementary to the mRNA of a protein the elimination of which improves a treatment outcome of an inherited or acquired disorder of myelin. In one example, the inherited or acquired disorder of myelin can be Pelizaeus-Merzbacher disease. In that case, the protein can be PLP1. To that end, the heterologous guide strand may comprise a nucleotide sequence having at least 70%, 80%, 85%, 90%, or 95% identity to one of SEQ ID NOs: 42 to 80, such as SEQ ID NO: 54 or SEQ ID NO: 75. In another example, the inherited or acquired disorder of myelin is multiple system atrophy. In that case, the targeted protein may be alpha-synuclein. To that end, the heterologous guide strand may comprise a nucleotide sequence having at least 70%, 80%, 85%, 90%, or 95% identity to one of SEQ ID NOs: 122 to 161. In a further example, the inherited or acquired disorder of myelin is Hypomyelination with Atrophy of the Basal Ganglia and Cerebellum (H-ABC). In that case, the targeted protein may be microtubule associated protein tubulin beta-4a. Accordingly, the heterologous guide strand may comprise a nucleotide sequence having at least 70%, 80%, 85%, 90%, or 95% identity to one of SEQ ID NOs: 204 to 244. Preferably, the nucleotide sequence has a length of 21-30 nucleotides.

In another aspect, the disclosure features an RNA molecule comprising a first RNA sequence and a second RNA sequence. The first sequence and the second RNA sequence are substantially complementary, wherein the first RNA sequence has a sequence length of at least 19 nucleotides and is at least 70%, 80%, 85%, 90% or 95% or 100% complementarity to one target sequence selected from the group consisting of SEQ ID NOs: 2-40, 82-121, and 163-203. In one embodiment, the target sequence comprises or is SEQ ID NO: 14 or 35. In one embodiment, the RNA molecule may be comprised in a pre-miRNA scaffold or a pri-miRNA scaffold, such as a human pri- or pre-miRNA scaffold derived from a human microRNA. Examples of the human microRNA include human mir-16-1, miR-21, miR-23a, miRNA-30a, miR-31, miR-122, miR-155, and miR-451. In one example, the human microRNA is miRNA-30a. Preferably, the human pri- or pre-miRNA scaffold does not include the native sequence for the native guide strand and native sequence for the native passenger strand of the human microRNA. In one example, the first strand is a heterologous guide strand inserted into the human pri- or pre-miRNA scaffold as a replacement for the native sequence of the native guide strand. In one embodiment, the RNA molecule comprises, or consists essentially of, or consists of the sequence of SEQ ID NO: 284 or 285 as shown in FIG. 11 or 12.

Within scope of this disclosure are (1) a polynucleotide encoding the RNA molecule described above, (2) an expression cassette or expression vector comprising the polynucleotide, and (3) a host cell comprising the polynucleotide or the expression cassette or expression vector. The expression cassette or expression vector may further comprise from 5′ to 3′ one or more of: a 5′ ITR, a promoter sequence region, a post-transcriptional regulatory element, a polyA signal sequence region, and a 3′ ITR. In some embodiments, the expression vector is a viral vector, such as an AAV vector. In one example, the AAV vector has a preferential tropism for an oligodendrocyte cell. Examples of such an AAV vector include AAV/Olig001, AAV/Olig002, and AAV/Olig003. Another example is AAV9 with six glutamate residues were inserted into the VP2 region such as that described in Powell S K et al. Mol Ther 28(5):1373-1380.

The RNA molecule, or the polynucleotide, or the expression cassette, or the expression vector, or the host cell described above can be used to treat a myelin-related disorder. Accordingly, the disclosure further provides a pharmaceutical composition comprising (a) one or more of the RNA molecule, the polynucleotide, the expression cassette, the expression vector, and the host cell, and (b) a pharmaceutical acceptable carrier.

Also provided is a method of treating a disorder of myelin. The method comprises administering to a subject in need thereof one or more of the above-described RNA molecule, polynucleotide, expression cassette, expression vector, and host cell. The subject can be a human. The administration can be carried out via injection. In some embodiments, the RNA molecule, or the polynucleotide, or the expression cassette, or the expression vector, or the host cell, or the pharmaceutical composition can be administered to a region of the central nervous system selected from the group consisting of brain parenchyma, spinal canal, subarachnoid space, a ventricle of the brain, cisterna magna and a combination thereof. The RNA molecule, or the polynucleotide, or the expression cassette, or the expression vector, or the host cell, or the pharmaceutical composition may be administered by a method selected from the group consisting of intraparenchymal administration, intrathecal administration, intracerebroventricular administration, intracisternal magna administration and a combination thereof. In some embodiments, the disorder is Pelizaeus-Merzbacher disease, multiple system atrophy, or Hypomyelination with Atrophy of the Basal Ganglia and Cerebellum.

The disclosure also provides similar RNA molecules, or polynucleotides, or expression cassettes, or expression vectors, or host cells, or pharmaceutical compositions, or methods for treating other myelin disorders in a subject in the same manner described above. In that case, the RNA molecules can reduce or inhibit the level or function of related genes, the gain of function of which leads to the disorders. Examples of the disorders and related genes include: Alexander disease with gain-of-function mutations in glial fibrillary acidic protein (GFAP), Mitchell disease with gain of function mutation in acyl-CoA oxidase 1 (ACOX1), autosomal dominant leukodystrophy with autonomic diseases (ADLD) with genomic duplications (or deletions upstream of the gene) of the lamin B1 gene (LMNB1) resulting in increased LMNB1 gene expression, central dysmyelinating leukodystrophy, Waardenburg syndrome, or Hirschsprung disease with duplication at 22q11.2q13, including SOX10, the gene encoding the transcription factor SOX-10, adult Polyglucosan Body Disease (APBD) with a mutation modifying the folding of glycogen branching enzyme (GBE1) leading to a gain of function, hereditary diffuse leukoencephalopathy with spheroids, with a gain-of-function mutation in CSF1R, the gene encoding the colony-stimulating factor 1 receptor, or Aicardi-Goutieres syndrome with gain-of-function mutations in IFIH1, the gene encoding Interferon-induced helicase C domain-containing protein 1.

Accordingly, the disclosure features an RNA molecule that reduces or inhibits the level or function of one of the genes described herein. The RNA molecule comprises a first RNA sequence and a second RNA sequence. The first sequence and the second RNA sequence are substantially complementary, wherein the first RNA sequence has a sequence length of at least 19 nucleotides and is at least 90% complementarity to an mRNA encoding one of the genes mentioned herein. In one embodiment, the RNA molecule may be comprised in a pre-miRNA scaffold or a pri-miRNA scaffold, such as a human pri- or pre-miRNA scaffold derived from a human microRNA. Examples of the human microRNA include human mir-16-1, miR-21, miR-23a, miRNA-30a, miR-31, miR-122, miR-155, and miR-451. Preferably, the human pri- or pre-miRNA scaffold does not include the native sequence for the native guide strand and native sequence for the native passenger strand of the human microRNA. In one example, the first strand is a heterologous guide strand inserted into the human pri- or pre-miRNA scaffold as a replacement for the native sequence of the native guide strand.

In other embodiments, the disorder is Canavan disease, Krabbe disease, Globoid cell leukodystrophy, X-linked adrenoleukodystrophy, Metachromatic leukodystrophy, hypomyelinating leukodystrophy-2, Niemann-Pick disease type C, 4H Leukodystrophy/Pol III-related leukodystrophy, Zellweger Spectrum Disorders, Childhood ataxia with central nervous system hypomyelination, Cerebrotendinous xanthomatosis, SOX10-associated peripheral demyelinating neuropathy, Adult Refsum disease, Autism Spectrum Disorder, Alzheimer's disease, Parkinson's disease, Fragile X syndrome, schizophrenia, multiple sclerosis, neuromyelitis optica, progressive multifocal leukoencephalopathy, encephalomyelitis, central pontine myelolysis, adrenoleukodystrophy, Wallerian Degeneration, optic neuritis, transverse myelitis, amyotrophic lateral sclerosis, Huntington's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Marie-Charcot-Tooth disease, or Bell's palsy.

The details of one or more embodiments of the disclosure are set forth in the description below. Other features, objectives, and advantages of the disclosure will be apparent from the description and from the claims.

### BRIEF DESCRIPTION OF THE DRAWINGS

FIG. 1 shows the 5′ Flanking sequence, the Stem Loop sequence with guide strand and passenger strand sequences depicted as red (bold and underlined) and blue (italic and underlined), the 3′ Flanking sequence, and the Stem-loop structure of human mir-16-1 NR_029486 (mbase accession MI0000070) (Han et al., 2006) with imperfect complementation resulting in nucleotide bulges.

FIG. 2 shows the 5′ Flanking sequence, the Stem Loop sequence with guide strand and passenger strand sequences depicted as red (bold and underlined) and blue (italic and underlined), the 3′ Flanking sequence, and the Stem-loop structure of human miR-21 NC_000017.11 (Yue et al., 2010) (mbase accession MI0000077) with imperfect complementation resulting in nucleotide bulges.

FIG. 3 shows the 5′ Flanking sequence, the Stem Loop sequence with guide strand and passenger strand sequences depicted as red (bold and underlined) and blue (italic and underlined), the 3′ Flanking sequence, and the Stem-loop structure of human miR-23a NR_029495 (mbase accession MI0000079) (van den Berg et al., 2016) with imperfect complementation resulting in nucleotide bulges.

FIG. 4 shows the 5′ Flanking sequence, the Stem Loop sequence with guide strand and passenger strand sequences depicted red (bold and underlined) and blue (italic and underlined), the 3′ Flanking sequence, and the Stem-loop structure of human miRNA-30a NR_029504 (mbase accession MI0000088) (Zeng et al., 2002) with imperfect complementation resulting in nucleotide bulges.

FIG. 5 shows the 5′ Flanking sequence, the Stem Loop sequence with guide strand and passenger strand sequences depicted red (bold and underlined) and blue (italic and underlined), the 3′ Flanking sequence, and the Stem-loop structure of human miR-31 NR_029505.1 (mbase accession MI0000089) (Ely et al., 2008) with imperfect complementation resulting in nucleotide bulges.

FIG. 6 shows the 5′ Flanking sequence, the Stem Loop sequence with guide strand and passenger strand sequences depicted as red (bold and underlined) and blue (italic and underlined), the 3′ Flanking sequence, and the Stem-loop structure of human miR-122 NR_029667 (mbase accession MI0000442) (Ely et al., 2008) with imperfect complementation resulting in nucleotide bulges.

FIG. 7 shows the 5′ Flanking sequence, the Stem Loop sequence with guide strand and passenger strand sequences depicted red (bold and underlined) and blue (italic and underlined), the 3′ Flanking sequence, and the Stem-loop structure of human miR-155 NR_030784 NC_000021.9 (mbase accession MI0000681) with imperfect complementation resulting in nucleotide bulges.

FIG. 8 shows the 5′ Flanking sequence, the Stem Loop sequence with the guide strand sequence depicted as red (bold and underlined), the 3′ Flanking sequence, and the Stem-loop structure of human miR-451 NR_029970 (mbase accession MI0001729) (Yoda et al., 2013) with imperfect complementation resulting in nucleotide bulges.

FIG. 9 shows an example of target region selection. Non-commercial web-based algorithm-based design tools were used to identify target regions in the coding sequence of the gene of interest. One target region is SEQ ID NO: 35, sequence shown in red (bold and underlined). The nucleotide sequence that is perfectly complementary to the target sequence was determined and is shown aligned 3′ to 5′ to demonstrate complementarity. Below the arrow, SEQ ID NO: 75 is shown in the 5′ to 3′ direction, in blue (italic and underlined).

FIG. 10 shows the 3′ and 5′ flanking regions (of 50-100 nucleotides) and the loop region (in black) of human miR-30a which was used as a backbone. The guide strand of human miR-30a is shown in red (bold and underlined). The 20-22 nucleotide sequence that is perfectly complementary to the PLP1 target sequence is substituted after 5′ flank sequence, followed by the loop sequence from the naturally occurring microRNA, the reverse complement of the 20-22 nucleotide guide strand (modified by deletion in positions 10-11 to create a bulge which permits preferential loading of the guide strand into the RISC complex, depicted in blue (bold and underlined)), then followed by the 3′ flank sequence.

FIG. 11 shows an example of the conserved secondary structures of the pre-miRNA polynucleotide of human miR-30a (SEQ ID NO: 283) and a modified miR-30a-PLP1 polynucleotide of the disclosure as predicted by mfold (SEQ ID NO: 284). The designed pre-miRNA miR-30a-PLP1 shares the same secondary structure as the native sequence (having the same framework regions, but different guide and strand sequences) and has a similar free energy (dG) as the native sequence. The resulting hairpin has a conserved loop region and contains the cleavage sites needed for Dicer to remove the loop and leave the dsRNA duplex. However, the two structures will target different mRNA molecules. In this case, miR-30a-PLP1 targets human PLP1 mRNA. The miR-30a-PLP1 sequence on the right contains the RNA sequence of SEQ ID NO: 75 where Ts are replaced by Us.

FIG. 12 shows another modified miR-30a-PLP1 polynucleotide of the disclosure as predicted by mfold (SEQ ID NO: 285). The designed pre-miRNA miR-30a-PLP1 shares the same secondary structure as the native sequence (having the same framework regions, but different guide and strand sequences) and has a similar free energy (dG) as the native sequence. The resulting hairpin has a conserved loop region and contains the cleavage sites needed for Dicer to remove the loop and leave the dsRNA duplex. In this case, the miR-30a-PLP1 contains 21-nucleotide RNA sequences from SEQ ID NO: 14 and SEQ ID 54 and targets human PLP1 mRNA.

### DETAILED DESCRIPTION OF THE INVENTION

This disclosure relates to compositions and methods for treating inherited and/or acquired disorders of myelin such as demyelination.

### 1. Myelin-Related Disorders

Myelin-related disorders include any diseases or conditions related to demyelination, insufficient myelination and remyelination, or dysmyelination in a subject. Such a disorder can be inherited or acquired or both. Demyelination in the CNS may occur in response to genetic mutation (leukodystrophies), autoimmune disease (e.g., multiple sclerosis), or trauma (e.g., traumatic brain injury, spinal cord injury, or ischemic stroke). Moreover, perturbation of myelin function may play a critical role in neurologic and psychiatric disorders such as Autism Spectrum Disorder (ASD), Alzheimer's disease (Nasrabady et al., 2018), Multiple System Atrophy (Wenning et al., 2008), Parkinson's disease (Bohnen and Albin, 2011), Fragile X syndrome (Filley, 2016), and schizophrenia (Najjar and Pearlman, 2015).

Leukodystrophies are a group of rare, primarily inherited neurological disorders that result from the abnormal production, processing, or development of myelin and are the result of genetic defects (mutations). Some forms are present at birth, while others may not produce symptoms until a child becomes a toddler. A few primarily affect adults. Leukodystrophies include Canavan disease (MIM #271900), Pelizaeus-Merzbacher disease (MIM #312080), Hypomyelination with Atrophy of the Basal Ganglia and Cerebellum (OMIM #612438), Krabbe disease (Globoid cell leukodystrophy, MIM #245200), X-linked adrenoleukodystrophy (MIM #300100), Metachromatic leukodystrophy (MLD, MIM #250100), Pelizaeus-Merzbacher-like disease (or hypomyelinating leukodystrophy-2, MIM #608804), Niemann-Pick disease type C (NPC, MIM #257220), Autosomal dominant leukodystrophy with autonomic diseases (ADLD, MIM #169500), 4H Leukodystrophy (Pol III-related leukodystrophy, MIM #607694), Zellweger Spectrum Disorders (ZSD, MIM #various), Childhood ataxia with central nervous system hypomyelination or CACH (also called vanishing white matter disease or VWMD, MIM #603896), Cerebrotendinous xanthomatosis (CTX, MIM #213700), Alexander disease: (AXD, MIM #203450), SOX10-associated peripheral demyelinating neuropathy, central dysmyelinating leukodystrophy, Waardenburg syndrome, and Hirschsprung disease (PCWH, MIM #609136), Adult polyglucosan body disease (APBD, MIM #263570), Hereditary diffuse leukoencephalopathy with spheroids (HDLS, MIM #221820), Aicardi-Goutieres syndrome (AGS, MIM #numerous), and Adult Refsum disease (MIM #266500).

### A. Pelizaeus-Merzbacher Disease

Pelizaeus-Merzbacher disease (PMD) is a rare, fatal, X-linked, recessive dysmyelinating leukodystrophy often presenting in the first year of life. Symptoms include hypotonia, nystagmus, and delayed developmental milestones, especially in motor function. PMD is progressive, with deteriorating coordination, motor abilities, and intellectual function, leading to early death, often before adulthood. There are approximately 3,500 patients in the US with PMD (Schmidt et al., 2020), and there are no effective therapies.

The most common (Elitt et al., 2020) form of PMD is caused by a duplication mutation of the gene encoding myelin protein proteolipid protein 1 (PLP1), one of the most abundant protein constituents of myelin. PLP1 contributes to the adhesion of the external membrane surfaces to create the unique architecture of the myelin sheath.

Overexpression of PLP1 protein in oligodendrocytes triggers dysfunction, prevents proper myelin formation, and results in extensive loss of myelinating oligodendrocytes in the CNS (Osório and Goldman, 2018). Reducing PLP1 expression to normal levels in patients with gene duplications can be used to restore oligodendrocyte function and improve outcome.

Overexpression of PLP1 is an example of a “toxic gain-of-function.” In this case, the overexpressed protein results in cellular toxicity. One way to address the overexpression of PLP1 is through the use of RNA interference to reduce expression of the PLP1 gene. This can be accomplished with a microRNA-based gene therapy. A single administration of an AAV vector delivering an expression cassette of a therapeutic miRNA precursor that targets PLP1 mRNA can be used to activate the endogenous mRNA silencing machinery to reduce PLP1 translation in oligodendrocytes. Moreover, since PLP1 overexpression is largely confined to oligodendrocytes, the use of AAV vectors with higher tropism for oligodendrocytes can be used to improve safety and therapeutic efficacy.

An antisense oligonucleotide strategy can be used to suppress the overexpression of PLP1 in jimpy mice (which model PMD by expressing abnormal PLP and recapitulating the cellular, molecular, and neurologic features seen in severe PMD). Administration of a single dose of PPL1-targeting antisense oligonucleotides in postnatal jimpy mice restored oligodendrocyte numbers, increased myelination, improved motor performance, normalized respiratory function and extended lifespan up to an eight-month end point (Elitt et al., 2020).

An miRNA strategy can also be used to reduce overexpression of PLP1 in Plp1-Tg mice, (a PMD model caused by PLP1 duplication). Direct intraparenchymal injection (into brain tissue, specifically, corpus striatum and internal capsule where oligodendrocytes are enriched) of an AAV vector with a transgene encoding a PLP1 mRNA targeting artificial miRNA (synthetic miR-155 harboring a PLP1 directed short hairpin) under the control of an oligodendrocyte specific (human CNP) prevented oligodendrocyte demise, restored myelin, and improved neurological phenotypes and survival in a mouse PMD model (Plp1-transgenic mice, a PLP1 duplication model) (Li et al., 2019).

Therapeutic approaches to PMD that combine AAV vectors that efficiently target oligodendrocytes with miRNA strategies for reducing PLP1 gene overexpression may be advantageous for treating PMD.

### B. Multiple System Atrophy

Multiple system atrophy (MSA) is a rare, fatal, and progressive neurodegenerative disorder characterized by symptoms that resemble the symptoms of Parkinson's disease, including slowness of movement, tremor, rigidity (stiffness), incoordination, and impaired speech. Symptoms typically appear when a person is between 50 and 60 years of age and progress rapidly. Most MSA patients die from the disease or its complications within 6 to 10 years of the onset of symptoms. There are approximately 15,000 to 50,000 patients in the US with MSA (Multiple System Atrophy Fact Sheet|National Institute of Neurological Disorders and Stroke, n.d.). While some medications provide symptomatic relief, there are no therapies that slow disease progression and there is no cure.

The pathology of MSA, like Parkinson's disease, is characterized by the accumulation and aggregation of the synaptic protein alpha-synuclein (a protein involved with neurotransmitter release). However, unlike Parkinson's disease, in which alpha-synuclein accumulates and aggregates in neurons, MSA is characterized by abnormal accumulations of alpha-synuclein, called glial cytoplasmic inclusions, within oligodendrocytes. Disruption of the oligodendrocyte-myelin-axon complex due to toxic accumulation alpha-synuclein in the form of glial cytoplasmic inclusions results in inflammation, demyelination, and subsequent neuronal loss.

Accumulation of alpha-synuclein protein in oligodendrocytes triggers dysfunction, prevents proper myelin formation, and leads to secondary neurodegeneration in the CNS (Marmion et al., 2021). Reducing the levels of alpha-synuclein in oligodendrocytes of MSA patients would be expected to restore suppress formation of glial cell inclusions and improve outcome.

One way to address the overexpression of alpha-synuclein is the use of RNA interference to reduce expression of the alpha-synuclein gene. This can be accomplished with a microRNA-based gene therapy. A single administration of an AAV vector delivering an expression cassette of a therapeutic miRNA precursor that targets alpha-synuclein mRNA would be expected to activate the endogenous mRNA silencing machinery to reduce alpha-synuclein translation and subsequent accumulation in oligodendrocytes. Moreover, since alpha-synuclein accumulation in MSA is largely confined to oligodendrocytes, the use of AAV vectors with higher tropism for oligodendrocytes would be expected to improve safety and therapeutic efficacy.

Recently, AAV-Olig001, an AAV vector with unique tropism for oligodendrocytes (Powell et al., 2016), was used to produce a model of MSA in mice. AAV-Olig001 with an α-synuclein transgene produced selective overexpression of α-synuclein in oligodendrocytes with >95% oligodendrocyte tropism in the dorsal striatum, resulting in demyelination and neuroinflammation similar to human MSA (Williams et al., 2020). Similarly, intrastriatal injection of AAV-Olig001 expressing the α-synuclein transgene in rhesus macaques resulted in widespread α-synuclein expression throughout the striatum. Demyelination was observed in the white matter tracts of the corpus callosum and striatum of AAV-Olig001-α-synuclein but not AAV-Olig001-GFP injected animals, similar to the human disease (Mandel et al., 2017).

Recently, Mavroeidi et al., showed that administration of alpha-synuclein to cultured mouse oligodendrocytes recruited endogenous oligodendrocyte alpha-synuclein in toxic aggregates (Mavroeidi et al., 2019). Similarly, endogenous oligodendrocyte alpha-synuclein was incorporated into pathological aggregates brought on by alpha-synuclein administration to mouse brains in vivo (Mavroeidi et al., 2019). Furthermore, this was mitigated in alpha-synuclein knock out mice. These results demonstrate that endogenous alpha-synuclein is necessary for the formation of intracellular alpha-synuclein aggregates. Manipulation of the expression of alpha-synuclein in oligodendrocytes may provide a rational approach to reduce alpha-synuclein accumulation in glial cytoplasmic inclusions and thereby delay or halt the rapid progression of MSA.

As disclosed herein, the use of novel therapeutic approaches to MSA that combine AAV vectors that efficiently target oligodendrocytes with miRNA strategies for reducing alpha-synuclein gene expression may be advantageous.

C. Hypomyelination with Atrophy of the Basal Ganglia and Cerebellum

Hypomyelination with Atrophy of the Basal Ganglia and Cerebellum (H-ABC) is a rare, autosomal dominant, dysmyelinating pediatric leukodystrophy. H-ABC typically presents in the toddler years, with symptoms such as dystonia, progressive gait impairment, and speech and cognitive deficits. Symptoms and progression of H-ABC are more severe when presenting in the first few months of life, but less severe when symptoms begin later in childhood. Magnetic resonance imaging (MRI) typically demonstrates a characteristic hypomyelination and atrophy of the caudate and putamen along with cerebellar atrophy (Simons et al., 2013). There are approximately 2,600 patients in the US with H-ABC (Schmidt et al., 2020), and there are no effective therapies.

H-ABC is caused by a toxic gain of function mutations in the TUBB4A gene, which encodes the microtubule associated protein tubulin beta-4a, which heterodimerizes with α-tubulin to form subunits that assemble into microtubules. Microtubules are the intracellular cables that help support the shape of the cell and move proteins to where they need to be. The tubulin beta-4a protein is a microtubule component highly expressed in mature oligodendrocytes. Mutations in the Tubb4a gene alter and disrupt microtubule dynamics and eventually result in the loss of oligodendrocytes (Curiel et al., 2017; Sase et al., 2020).

Overexpression of tubulin beta-4a protein alters microtubule dynamics in oligodendrocytes and triggers dysfunction, prevents proper myelin formation, and results in extensive loss of myelinating oligodendrocytes in the CNS (Curiel et al., 2017; Sase et al., 2020). Normalizing tubulin beta-4a protein expression in patients can be used to restore oligodendrocyte function and improve outcome.

The overexpression of tubulin beta-4a protein is another example of a “toxic gain-of-function.” In this case, the overexpressed protein results in disruption of microtubule dynamics leading to cellular toxicity. One way to address the overexpression of tubulin beta-4a protein is through the use of RNA interference to reduce expression of the TUBB4A gene. This can be accomplished with a microRNA-based gene therapy. A single administration of an AAV vector delivering an expression cassette of a therapeutic miRNA precursor that targets TUBB4A mRNA can be used to activate the endogenous mRNA silencing machinery to reduce TUBB4A translation in oligodendrocytes. Moreover, since TUBB4A overexpression is largely confined to oligodendrocytes, the use of AAV vectors with higher tropism for oligodendrocytes can be used to improve safety and therapeutic efficacy.

The most common mutation is patients with H-ABC is TUBB4A^{D249N}. Like all proteins, tubulin beta-4a protein is composed of amino acids linked together like a chain and then folded into a precise configuration. Mutations in the genes encoding proteins can result in the incorporation of an incorrect amino acid into the chain. In this case, tubulin beta-4a normally has the amino acid aspartate at the 249^{th} position in the chain. The mutation results in a switch to the amino acid asparagine at this position. This small change affects how the protein functions, often in large ways.

Recently, Sase et al., developed a mouse model of H-ABC in which mice have the same mutation in the mouse TUBB4A gene (Sase et al., 2020). These mice (Tubb4a^{D249N/D249N} mice) exhibited a progressive motor dysfunction, with abnormal walking gait, poor coordination, and involuntary movements such as twitching and reduced reflexes, similar to H-ABC patients. Tubb4a^{D249N/D249N} also exhibit shortened survival relative to controls, loss of myelin staining relative to control mice, alterations in the behavior and formation of microtubules, and a dramatic loss of oligodendrocytes. These results demonstrate that Tubb4a^{D249N/D249N} mice share many similar symptoms and pathologies with H-ABC disease in humans. Therapeutic approaches to H-ABC that combine AAV vectors that efficiently target oligodendrocytes with miRNA and/or gene replacement strategies for normalizing TUBB4A gene expression may be advantageous.

The present disclosure employs viral vectors such as AAV vectors to deliver therapeutic nucleic acids, such as siRNAs, targeting one or more genes or RNAs encoding proteins of toxic gain-of-function, into cells with high efficiency. In some embodiments, the AAV vectors encoding RNAi molecules, e.g., siRNA molecules of the present disclosure may increase the delivery of active agents into oligodendrocytes. The therapeutic nucleic acids or polynucleotides may be able to inhibit gene expression (e.g., mRNA level) of a toxic gain-of-function protein significantly inside cells; therefore, ameliorating defects induced or caused by the protein inside the cells such as aggregation of protein and formation of inclusions.

Such inhibitory nucleic acids, e.g., siRNAs. may be used for treating various inherited and/or acquired disorders of myelin such as demyelination. According to the present disclosure, methods for treating and/or ameliorating the disorder in a patient comprises administering to the patient an effective amount of at least one therapeutic nucleic acid (e.g., a polynucleotide encoding one or more siRNA duplexes) into cells and allowing the inhibition/silence of the gene expression.

### 2. Nucleic Acids

Certain aspects of the disclosure provide one or more inhibitory nucleic acids (e.g., inhibitory RNA molecules), polynucleotides encoding such inhibitory nucleic acids, and transgenes engineered to express such inhibitory nucleic acids. The one or more inhibitory nucleic acids may target the same gene (e.g., hybridize or specifically bind to a same mRNA sequence or different mRNA sequences of the same gene) or different genes (e.g., hybridize or specifically bind to mRNAs of different genes).

### A. Inhibitory Nucleic Acids

An inhibitory nucleic acid refers to a nucleic acid that can bind to a target nucleic acid (e.g., a target RNA) in a cell and reduce or inhibit the level or function of the target nucleic acid in the cell. Example of the inhibitory nucleic acid include antisense oligonucleotides, ribozymes, external guide sequence (EGS) oligonucleotides, small interfering (si)RNA compounds, single- or double-stranded RNA interference (RNAi) compounds, modified bases/locked nucleic acids (LNAs), peptide nucleic acids (PNAs), and other oligomeric compounds or oligonucleotide mimetics that specifically hybridize to at least a portion of a target nucleic acid and modulate its level or function. In some embodiments, the inhibitory nucleic acid can be an antisense RNA, an antisense DNA, a chimeric antisense oligonucleotide, an antisense oligonucleotide comprising modified linkages, an interference RNA (iRNA), a short or small interfering RNA (siRNA), a micro RNA or micro interfering RNA (miRNA), a small temporal RNA (stRNA), a short hairpin RNA (shRNA), a small RNA-induced gene activation agent (RNAa), a small activating RNA (saRNA), or combinations thereof. In some examples, the inhibitory nucleic acid is an inhibitory RNA molecule that mediates RNA interference.

RNA interference (RNAi) is a process discovered in 1998 (Fire et al., 1998) by which cells regulate gene expression. A double-stranded RNA (dsRNA) in the cell cytoplasm triggers the RNAi pathway in which the double-stranded RNA is processed into small double-stranded fragments of approximately 21-23 nucleotides in length by the RNAse III-like enzyme DICER. These double-stranded fragments are integrated into a multi-subunit protein called the RNA-induced silencing complex (RISC). The RISC contains Argonaute proteins that unwind the double-stranded fragment into a passenger strand that is removed from the complex and a guide strand that is complementary to a target sequence in a specific mRNA and which directs the RISC complex to cleave or suppress the translation of the specific target mRNA molecule (Kotowska-Zimmer et al., 2021). In this way the gene that encoded the mRNA molecule is rendered essentially inactive or “silenced.”

RNAi technology may employ three kinds of tools: synthetic siRNAs, vector-based shRNAs, and artificial miRNAs (amiRNAs). Synthetic siRNAs are exogenous double stranded RNAs that must be delivered into cells and must overcome stability and pharmacokinetic challenges. shRNAs are artificial RNA molecules with a tight hairpin loop structure that are delivered to cells using plasmids or viral expression vectors. shRNAs are typically transcribed from strong pol III promoters (e.g., U6 or H1) and enter the RNAi pathway as hairpins. However, transcription driven by strong pol III promoters can produce supraphysiologic levels of shRNA that saturate the endogenous miRNA biogenesis machinery, resulting in toxicity. AmiRNAs embed a target-specific shRNA insert in a scaffold based on a natural primary miRNA (pri-miRNA). This ensures proper processing and transport similar to endogenous miRNAs, resulting in lower toxicity (Kotowska-Zimmer et al., 2021).

In some embodiments of this disclosure, the inhibitory RNA molecule can be an siRNA, a miRNA (including an amiRNA), or an shRNA.

An siRNA is known in the art as a double-stranded RNA molecule of approximately 19-25 (e.g., 19-23) base pairs in length that induces RNAi in a cell. In some embodiments, the siRNA sequence can also be inserted into an artificial miRNA scaffold (“shmiRNA”).

An shRNA is known in the art as an RNA molecule comprising approximately 19-25 (e.g., 19-23) base pairs of double stranded RNA linked by a short loop (e.g., about 4-11 nucleotides) that induces RNAi in a cell.

An miRNA is known in the art as an RNA molecule that induces RNAi in a cell comprising a short (e.g., 19-25 base pairs) sequence of double-stranded RNA linked by a loop and containing one or more additional sequences of double-stranded RNA comprising one or more bulges (e.g., mis-paired or unpaired base pairs). As used herein, the term “miRNA” encompasses endogenous miRNAs as well as exogenous or heterologous miRNAs. In some embodiments, “miRNA” may refer to a pri-miRNA or a pre-miRNA. During miRNA processing, a pri-miRNA transcript is produced. The pri-miRNA is processed by Drosha-DGCR8 to produce a pre-miRNA by excising one or more sequences to leave a pre-miRNA with a 5′ flanking region, a guide strand, a loop region, a non-guide strand, and a 3′ flanking region; or a 5′ flanking region, a non-guide strand, a loop region, a guide strand, and a 3′ flanking region. The pre-miRNA is then exported to the cytoplasm and processed by Dicer to yield a siRNA with a guide strand and a non-guide (or passenger) strand. The guide strand is then used by the RISC complex to catalyze gene silencing, e.g., by recognizing a target RNA sequence complementary to the guide strand. Further description of miRNAs may be found, e.g., in WO 2008/150897. The recognition of a target sequence by a miRNA is primarily determined by pairing between the target and the miRNA seed sequence, e.g., nucleotides 1-8 (5′ to 3′) of the guide strand (see, e.g., Boudreau, R. L. et al. (2013) Nucleic Acids Res. 41:e9).

In some embodiments of this disclosure, an inhibitory RNA molecule forms a hairpin structure. Generally, hairpin-forming RNAs are arranged into a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA may have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue.

In some embodiments, an inhibitory RNA molecule is processed in a cell (or subject) to form a “mature miRNA”. Mature miRNA is the result of a multistep pathway which is initiated through the transcription of primary miRNA from its miRNA gene or intron, by RNA polymerase II or III generating the initial precursor molecule in the biological pathway resulting in miRNA. Once transcribed, pri-miRNA (often over a thousand nucleotides long with a hairpin structure) is processed by the Drosha enzyme which cleaves pri-miRNA near the junction between the hairpin structure and the ssRNA, resulting in precursor miRNA (pre-miRNA). The pre-miRNA is exported to the cytoplasm where is further reduced by Dicer enzyme at the pre-miRNA loop, resulting in duplexed miRNA strands.

Of the two strands of a miRNA duplex, one arm, the guide strand (miR), is typically found in higher concentrations and binds and associates with the Argonaute protein which is eventually loaded into the RNA-inducing silencing complex. The guide strand miRNA-RISC complex helps regulates gene expression by binding to its complementary sequence of mRNA, often in the 3′ UTR of the mRNA. The non-guide strand of the miRNA duplex is known as the passenger strand and is often degraded, but may persist and also act either intact or after partial degradation to have a functional role in gene expression.

In some embodiments, a transgene is engineered to express an inhibitory nucleic acid (e.g., an miRNA) having a guide strand that targets a human gene. “Targeting” refers to hybridization or specific binding of an inhibitory nucleic acid to its cognate (e.g., complementary) sequence on a target gene (e.g., mRNA transcript of a target gene). In some embodiments, an inhibitory nucleic acid that targets a gene transcript shares a region of complementarity with the target gene that is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, a region of complementarity is more than 30 nucleotides in length.

Typically, the guide strand may target a human gene transcript associated with a disease or disorder of myelin. Examples include that for PLP1 (associated with PMD), SNCA (associated with MSA), or TUBB4A (associated with H-ABC). In some embodiments, a guide strand that targets any of these gene transcripts is encoded by an isolated nucleic acid comprising the sequence set forth below.

Shown below is a sequence encoding Homo sapiens proteolipid protein 1 (PLP1), transcript variant 1 mRNA.

NCBI Reference Sequence: PLP1 (NM_000533.5)
coding sequence
(SEQ ID NO: 1)
ATGGGCTTGTTAGAGTGCTGTGCAGTGGCACTGTTCTGTGGCTGTGGAC
ATGAAGCCCTCACTGGCACAGAAAAGCTAATTGAGACCTATTTCTCCAA
AAACTACCAAGACTATGAGTATCTCATCAATGTGATCCATGCCTTCCAG
TATGTCATCTATGGAACTGCCTCTTTCTTCTTCCTTTATGGGGCCCTCC
TGCTGGCTGAGGGCTTCTACACCACCGGCGCAGTCAGGCAGATCTTTGG
CGACTACAAGACCACCATCTGCGGCAAGGGCCTGAGCGCAACGGTAACA
GGGGGCCAGAAGGGGAGGGGTTCCAGAGGCCAACATCAAGCTCATTCTT
TGGAGCGGGTGTGTCATTGTTTGGGAAAATGGCTAGGACATCCCGACAA
GTTTGTGGGCATCACCTATGCCCTGACCGTTGTGTGGCTCCTGGTGTTT
GCCTGCTCTGCTGTGCCTGTGTACATTTACTTCAACACCTGGACCACCT
GCCAGTCTATTGCCTTCCCCAGCAAGACCTCTGCCAGTATAGGCAGTCT
CTGTGCTGATGCCAGAATGTATGGTGTTCTCCCATGGAATGCTTTCCCT
GGCAAGGTTTGTGGCTCCAACCTTCTGTCCATCTGCAAAACAGCTGAGT
TCCAAATGACCTTCCACCTGTTTATTGCTGCATTTGTGGGGGCTGCAGC
TACACTGGTTTCCCTGCTCACCTTCATGATTGCTGCCACTTACAACTTT
GCCGTCCTTAAACTCATGGGCCGAGGCACCAAGTTCTGA

Shown in Table 1 below are exemplary target sequences and guide strands for targeting PLP1.

TABLE 1
PLP1 Target Sequences

| SEQ | | SEQ | |
|---|---|---|---|
| ID | | ID | |
| NO: | Target Sequence | NO: | Guide Strand |
| 2 | CTACCAAGACTATGAGTATCT | 42 | AGATACTCATAGTCTTGGTAG |
| 3 | ACTACCAAGACTATGAGTATC | 43 | GATACTCATAGTCTTGGTAGT |
| 4 | CAAGACTATGAGTATCTCATC | 44 | GATGAGATACTCATAGTCTTG |
| 5 | GACTATGAGTATCTCATCAAT | 45 | ATTGATGAGATACTCATAGTC |
| 6 | ACTATGAGTATCTCATCAATG | 46 | CATTGATGAGATACTCATAGT |
| 7 | GAGTATCTCATCAATGTGATC | 47 | GATCACATTGATGAGATACTC |
| 8 | TGATCCATGCCTTCCAGTATG | 48 | CATACTGGAAGGCATGGATCA |
| 9 | CCTTCCAGTATGTCATCTATG | 49 | CATAGATGACATACTGGAAGG |
| 10 | CAGTATGTCATCTATGGAACT | 50 | AGTTCCATAGATGACATACTG |
| 11 | ATCTATGGAACTGCCTCTTTC | 51 | GAAAGAGGCAGTTCCATAGAT |
| 12 | CCTCTTTCTTCTTCCTTTATG | 52 | CATAAAGGAAGAAGAAAGAGG |
| 13 | TGCCTCTTTCTTCTTCCTTTA | 53 | TAAAGGAAGAAGAAAGAGGCA |
| 14 | GCCTCTTTCTTCTTCCTTTAT | 54 | ATAAAGGAAGAAGAAAGAGGC |
| 15 | CCTCTTTCTTCTTCCTTTATG | 55 | CATAAAGGAAGAAGAAAGAGG |
| 16 | GGCGACTACAAGACCACCATC | 56 | GATGGTGGTCTTGTAGTCGCC |
| 17 | GCGACTACAAGACCACCATCT | 57 | AGATGGTGGTCTTGTAGTCGC |
| 18 | AGGCCAACATCAAGCTCATTC | 58 | GAATGAGCTTGATGTTGGCCT |
| 19 | CAACATCAAGCTCATTCTTTG | 59 | CAAAGAATGAGCTTGATGTTG |
| 20 | TTTGGAGCGGGTGTGTCATTG | 60 | CAATGACACACCCGCTCCAAA |
| 21 | GAGCGGGTGTGTCATTGTTTG | 61 | CAAACAATGACACACCCGCTC |
| 22 | GGTGTGTCATTGTTTGGGAAA | 62 | TTTCCCAAACAATGACACACC |
| 23 | GGCTAGGACATCCCGACAAGT | 63 | ACTTGTCGGGATGTCCTAGCC |
| 24 | AGGACATCCCGACAAGTTTGT | 64 | ACAAACTTGTCGGGATGTCCT |
| 25 | AGTTTGTGGGCATCACCTATG | 65 | CATAGGTGATGCCCACAAACT |
| 26 | GCTCTGCTGTGCCTGTGTACA | 66 | TGTACACAGGCACAGCAGAGC |
| 27 | GCTGTGCCTGTGTACATTTAC | 67 | GTAAATGTACACAGGCACAGC |
| 28 | CTGCTGTGCCTGTGTACATTT | 68 | AAATGTACACAGGCACAGCAG |
| 29 | CTGTGCCTGTGTACATTTACT | 69 | AGTAAATGTACACAGGCACAG |
| 30 | TGCTGTGCCTGTGTACATTTA | 70 | TAAATGTACACAGGCACAGCA |
| 31 | GCTGTGCCTGTGTACATTTAC | 71 | GTAAATGTACACAGGCACAGC |
| 32 | CTGTGTACATTTACTTCAACA | 72 | TGTTGAAGTAAATGTACACAG |
| 33 | TGGACCACCTGCCAGTCTATT | 73 | AATAGACTGGCAGGTGGTCCA |
| 34 | AGCAAGACCTCTGCCAGTATA | 74 | TATACTGGCAGAGGTCTTGCT |
| 35 | ATGACCTTCCACCTGTTTATT | 75 | AATAAACAGGTGGAAGGTCAT |
| 36 | TGACCTTCCACCTGTTTATTG | 76 | CAATAAACAGGTGGAAGGTCA |
| 37 | CCACCTGTTTATTGCTGCATT | 77 | AATGCAGCAATAAACAGGTGG |
| 38 | CCTGTTTATTGCTGCATTTGT | 78 | ACAAATGCAGCAATAAACAGG |
| 39 | TACAACTTTGCCGTCCTTAAA | 79 | TTTAAGGACGGCAAAGTTGTA |
| 40 | ACAACTTTGCCGTCCTTAAAC | 80 | GTTTAAGGACGGCAAAGTTGT |

Shown below is a sequence encoding the Homo sapiens synuclein alpha (SNCA), transcript variant 1, mRNA.

NCBI Reference Sequence: NM_000345.4
coding sequence
(SEQ ID NO: 81)
ATGGATGTATTCATGAAAGGACTTTCAAAGGCCAAGGAGGGAGTTGTGG
CTGCTGCTGAGAAAACCAAACAGGGTGTGGCAGAAGCAGCAGGAAAGAC
AAAAGAGGGTGTTCTCTATGTAGGCTCCAAAACCAAGGAGGGAGTGGTG
CATGGTGTGGCAACAGTGGCTGAGAAGACCAAAGAGCAAGTGACAAATG
TTGGAGGAGCAGTGGTGACGGGTGTGACAGCAGTAGCCCAGAAGACAGT
GGAGGGAGCAGGGAGCATTGCAGCAGCCACTGGCTTTGTCAAAAAGGAC
CAGTTGGGCAAGAATGAAGAAGGAGCCCCACAGGAAGGAATTCTGGAAG
ATATGCCTGTGGATCCTGACAATGAGGCTTATGAAATGCCTTCTGAGGA
AGGGTATCAAGACTACGAACCTGAAGCCTAA

Shown in Table 2 below are exemplary target sequences and guide strands for targeting human alpha synuclein.

TABLE 2
human alpha synuclein target sequences

| SEQ | | SEQ | |
|---|---|---|---|
| ID | | ID | |
| NO: | Target Sequence | NO: | Guide Strand |
| 82 | GGATGTATTCATGAAAGGACT | 122 | AGTCCTTTCATGAATACATCC |
| 83 | ATGTATTCATGAAAGGACTTT | 123 | AAAGTCCTTTCATGAATACAT |
| 84 | TGTATTCATGAAAGGACTTTC | 124 | GAAAGTCCTTTCATGAATACA |
| 85 | AGTTGTGGCTGCTGCTGAGAA | 125 | TTCTCAGCAGCAGCCACAACT |
| 86 | GTTGTGGCTGCTGCTGAGAAA | 126 | TTTCTCAGCAGCAGCCACAAC |
| 87 | TGTGGCAGAAGCAGCAGGAAA | 127 | TTTCCTGCTGCTTCTGCCACA |
| 88 | TGGCAGAAGCAGCAGGAAAGA | 128 | TCTTTCCTGCTGCTTCTGCCA |
| 89 | AGAGGGTGTTCTCTATGTAGG | 129 | CCTACATAGAGAACACCCTCT |
| 90 | CAGTGGCTGAGAAGACCAAAG | 130 | CTTTGGTCTTCTCAGCCACTG |
| 91 | GACCAAAGAGCAAGTGACAAA | 131 | TTTGTCACTTGCTCTTTGGTC |
| 92 | CCAAAGAGCAAGTGACAAATG | 132 | CATTTGTCACTTGCTCTTTGG |
| 93 | CAAAGAGCAAGTGACAAATGT | 133 | ACATTTGTCACTTGCTCTTTG |
| 94 | ACCAAAGAGCAAGTGACAAAT | 134 | ATTTGTCACTTGCTCTTTGGT |
| 95 | CCAAAGAGCAAGTGACAAATG | 135 | CATTTGTCACTTGCTCTTTGG |
| 96 | CAAAGAGCAAGTGACAAATGT | 136 | ACATTTGTCACTTGCTCTTTG |
| 97 | AGAGCAAGTGACAAATGTTGG | 137 | CCAACATTTGTCACTTGCTCT |
| 98 | GCAAGTGACAAATGTTGGAGG | 138 | CCTCCAACATTTGTCACTTGC |
| 99 | AGTGACAAATGTTGGAGGAGC | 139 | GCTCCTCCAACATTTGTCACT |
| 100 | GGACCAGTTGGGCAAGAATGA | 140 | TCATTCTTGCCCAACTGGTCC |
| 101 | AGGACCAGTTGGGCAAGAATG | 141 | CATTCTTGCCCAACTGGTCCT |
| 102 | GACCAGTTGGGCAAGAATGAA | 142 | TTCATTCTTGCCCAACTGGTC |
| 103 | GTTGGGCAAGAATGAAGAAGG | 143 | CCTTCTTCATTCTTGCCCAAC |
| 104 | GGGCAAGAATGAAGAAGGAGC | 144 | GCTCCTTCTTCATTCTTGCCC |
| 105 | GGAAGGAATTCTGGAAGATAT | 145 | ATATCTTCCAGAATTCCTTCC |
| 106 | GAAGGAATTCTGGAAGATATG | 146 | CATATCTTCCAGAATTCCTTC |
| 107 | GGAAGATATGCCTGTGGATCC | 147 | GGATCCACAGGCATATCTTCC |
| 108 | GCCTGTGGATCCTGACAATGA | 148 | TCATTGTCAGGATCCACAGGC |
| 109 | TGCCTGTGGATCCTGACAATG | 149 | CATTGTCAGGATCCACAGGCA |
| 110 | GATCCTGACAATGAGGCTTAT | 150 | ATAAGCCTCATTGTCAGGATC |
| 111 | ATCCTGACAATGAGGCTTATG | 151 | CATAAGCCTCATTGTCAGGAT |
| 112 | TCCTGACAATGAGGCTTATGA | 152 | TCATAAGCCTCATTGTCAGGA |
| 113 | CCTGACAATGAGGCTTATGAA | 153 | TTCATAAGCCTCATTGTCAGG |
| 114 | GACAATGAGGCTTATGAAATG | 154 | CATTTCATAAGCCTCATTGTC |
| 115 | CTGACAATGAGGCTTATGAAA | 155 | TTTCATAAGCCTCATTGTCAG |
| 116 | TGACAATGAGGCTTATGAAAT | 156 | ATTTCATAAGCCTCATTGTCA |
| 117 | GACAATGAGGCTTATGAAATG | 157 | CATTTCATAAGCCTCATTGTC |
| 118 | AGGAAGGGTATCAAGACTACG | 158 | CGTAGTCTTGATACCCTTCCT |
| 119 | AGGGTATCAAGACTACGAACC | 159 | GGTTCGTAGTCTTGATACCCT |
| 120 | TCAAGACTACGAACCTGAAGC | 160 | GCTTCAGGTTCGTAGTCTTGA |
| 121 | CTACGAACCTGAAGCCTAA | 161 | TTAGGCTTCAGGTTCGTAG |

Shown below is a sequence encoding the Homo sapiens tubulin beta 4A class IVa (TUBB4A), transcript variant 1; NCBI Reference Sequence: NM_001289123.2 (SEQ ID NO: 162).

ATGAGAAGGGGGGCTGCGGACCGAGAAACTGAGCGGCTCCCGGGGGCGCA
GGGACCGTGCTCCGCCGTCTCCGCCGCATCTTCCACCCTCGCCGCCGCCG
CAGCTCCCCGCGCTCGTGCCACCGCCGCCGCGTCCACCCTCAGCGCCACC
GCCATGCGGGAGATCGTGCACCTGCAGGCCGGCCAGTGCGGCAACCAGAT
CGGGGCCAAGTTTTGGGAGGTTATCAGTGACGAACATGGCATCGACCCCA
CAGGCACATACCATGGGGACAGTGACCTGCAACTGGAGAGGATCAACGTG
TACTACAACGAGGCCACAGGAGGAAATTATGTCCCCAGAGCGGTGCTGGT
GGACCTGGAACCCGGCACCATGGACTCTGTCCGTTCTGGCCCCTTCGGTC
AGATCTTTCGGCCGGACAACTTCGTGTTTGGCCAATCCGGAGCCGGCAAC
AACTGGGCAAAGGGGCACTACACGGAGGGCGCAGAGCTGGTGGACGCTGT
CCTGGACGTAGTCCGGAAGGAGGCCGAGAGCTGCGACTGCCTTCAGGGCT
TCCAGCTGACCCACTCGCTGGGGGGTGGCACGGGGTCCGGAATGGGCACG
CTGCTCATCAGTAAGATCCGCGAGGAGTTCCCAGACCGCATCATGAACAC
CTTCAGCGTGGTGCCCTCGCCCAAAGTGTCAGACACGGTGGTGGAGCCCT
ACAACGCCACGCTGTCTGTGCACCAGCTGGTGGAGAATACGGATGAGACC
TACTGCATCGACAACGAGGCACTCTACGACATCTGTTTCCGCACCCTCAA
GCTGACCACCCCCACCTACGGGGACCTCAACCACCTGGTGTCGGCCACCA
TGAGCGGGGTCACCACCTGCCTGCGCTTCCCGGGCCAGCTGAACGCCGAC
CTGCGCAAGCTGGCCGTCAACATGGTTCCCTTTCCTCGCCTGCACTTCTT
CATGCCCGGCTTCGCACCCCTGACCAGCCGGGGCAGCCAGCAGTACCGGG
CCCTGACGGTGCCCGAGCTCACCCAGCAGATGTTCGATGCCAAGAACATG
ATGGCGGCGTGCGACCCGCGCCACGGCCGCTACCTGACCGTGGCCGCCGT
GTTCCGGGGCCGCATGTCCATGAAGGAGGTGGACGAGCAGATGCTGAGCG
TGCAGAGCAAGAACAGCAGCTACTTCGTGGAGTGGATCCCCAACAACGTG
AAGACGGCCGTGTGCGACATCCCGCCCCGCGGCCTGAAGATGGCCGCGAC
CTTCATCGGCAACAGCACGGCCATCCAGGAGCTGTTCAAGCGCATCTCCG
AGCAGTTCACGGCCATGTTCCGGCGCAAGGCCTTCTTGCACTGGTACACG
GGCGAGGGCATGGACGAGATGGAGTTCACCGAGGCCGAGAGCAACATGAA
TGACCTGGTATCTGAGTACCAGCAGTACCAGGACGCCACGGCCGAGGAGG
GCGAGTTCGAGGAGGAGGCGGAGGAGGAGGTGGCCTAG

Shown in Table 3 below are exemplary target sequences and guide strands for targeting Human TUBB4A.

TABLE 3
Human TUBB4A target sequences

| SEQ | | SEQ | |
|---|---|---|---|
| ID | | ID | |
| NO: | Target Sequence | NO: | Guide Strand |
| 163 | GGGAGGTTATCAGTGACGAAC | 204 | GTTCGTCACTGATAACCTCCC |
| 164 | GGAGGTTATCAGTGACGAACA | 205 | TGTTCGTCACTGATAACCTCC |
| 165 | GGTTATCAGTGACGAACATGG | 206 | CCATGTTCGTCACTGATAACC |
| 166 | GAGGTTATCAGTGACGAACAT | 207 | ATGTTCGTCACTGATAACCTC |
| 167 | AGGTTATCAGTGACGAACATG | 208 | CATGTTCGTCACTGATAACCT |
| 168 | CCTGCAACTGGAGAGGATCAA | 209 | TTGATCCTCTCCAGTTGCAGG |
| 169 | CTGCAACTGGAGAGGATCAAC | 210 | GTTGATCCTCTCCAGTTGCAG |
| 170 | TGGAGAGGATCAACGTGTACT | 211 | AGTACACGTTGATCCTCTCCA |
| 171 | GGAGAGGATCAACGTGTACTA | 212 | TAGTACACGTTGATCCTCTCC |
| 172 | GAGAGGATCAACGTGTACTAC | 213 | GTAGTACACGTTGATCCTCTC |
| 173 | AGAGGATCAACGTGTACTACA | 214 | TGTAGTACACGTTGATCCTCT |
| 174 | GGATCAACGTGTACTACAACG | 215 | CGTTGTAGTACACGTTGATCC |
| 175 | GAGGATCAACGTGTACTACAA | 216 | TTGTAGTACACGTTGATCCTC |
| 176 | ACGAGGCCACAGGAGGAAATT | 217 | AATTTCCTCCTGTGGCCTCGT |
| 177 | CGAGGCCACAGGAGGAAATTA | 218 | TAATTTCCTCCTGTGGCCTCG |
| 178 | GAGGCCACAGGAGGAAATTAT | 219 | ATAATTTCCTCCTGTGGCCTC |
| 179 | AGGCCACAGGAGGAAATTATG | 220 | CATAATTTCCTCCTGTGGCCT |
| 180 | GGCCACAGGAGGAAATTATGT | 221 | ACATAATTTCCTCCTGTGGCC |
| 181 | CCAGAGCGGTGCTGGTGGACC | 222 | GGTCCACCAGCACCGCTCTGG |
| 182 | GGTGCTGGTGGACCTGGAACC | 223 | GGTTCCAGGTCCACCAGCACC |
| 183 | AGATCTTTCGGCCGGACAACT | 224 | AGTTGTCCGGCCGAAAGATCT |
| 184 | CAACTTCGTGTTTGGCCAATC | 225 | GATTGGCCAAACACGAAGTTG |
| 185 | GCACGCTGCTCATCAGTAAGA | 226 | TCTTACTGATGAGCAGCGTGC |
| 186 | CACGCTGCTCATCAGTAAGAT | 227 | ATCTTACTGATGAGCAGCGTG |
| 187 | CGGTGGTGGAGCCCTACAACG | 228 | CGTTGTAGGGCTCCACCACCG |
| 188 | TGCACCAGCTGGTGGAGAATA | 229 | TATTCTCCACCAGCTGGTGCA |
| 189 | GCTGGTGGAGAATACGGATGA | 230 | TCATCCGTATTCTCCACCAGC |
| 190 | CGGATGAGACCTACTGCATCG | 231 | CGATGCAGTAGGTCTCATCCG |
| 191 | GAGACCTACTGCATCGACAAC | 232 | GTTGTCGATGCAGTAGGTCTC |
| 192 | AGACCTACTGCATCGACAACG | 233 | CGTTGTCGATGCAGTAGGTCT |
| 193 | CCTACTGCATCGACAACGAGG | 234 | CCTCGTTGTCGATGCAGTAGG |
| 194 | TCGACAACGAGGCACTCTACG | 235 | CGTAGAGTGCCTCGTTGTCGA |
| 195 | ACGAGGCACTCTACGACATCT | 236 | AGATGTCGTAGAGTGCCTCGT |
| 196 | GCACTCTACGACATCTGTTTC | 237 | GAAACAGATGTCGTAGAGTGC |
| 197 | CCGTCAACATGGTTCCCTTTC | 238 | GAAAGGGAACCATGTTGACGG |
| 198 | GCTACTTCGTGGAGTGGATCC | 239 | GGATCCACTCCACGAAGTAGC |
| 199 | CAGGAGCTGTTCAAGCGCATC | 240 | GATGCGCTTGAACAGCTCCTG |
| 200 | AGGCCTTCTTGCACTGGTACA | 241 | TGTACCAGTGCAAGAAGGCCT |
| 201 | GAGGCCGAGAGCAACATGAAT | 242 | ATTCATGTTGCTCTCGGCCTC |
| 202 | AGGCCGAGAGCAACATGAATG | 243 | CATTCATGTTGCTCTCGGCCT |
| 203 | CAACATGAATGACCTGGTATC | 244 | GATACCAGGTCATTCATGTTG |

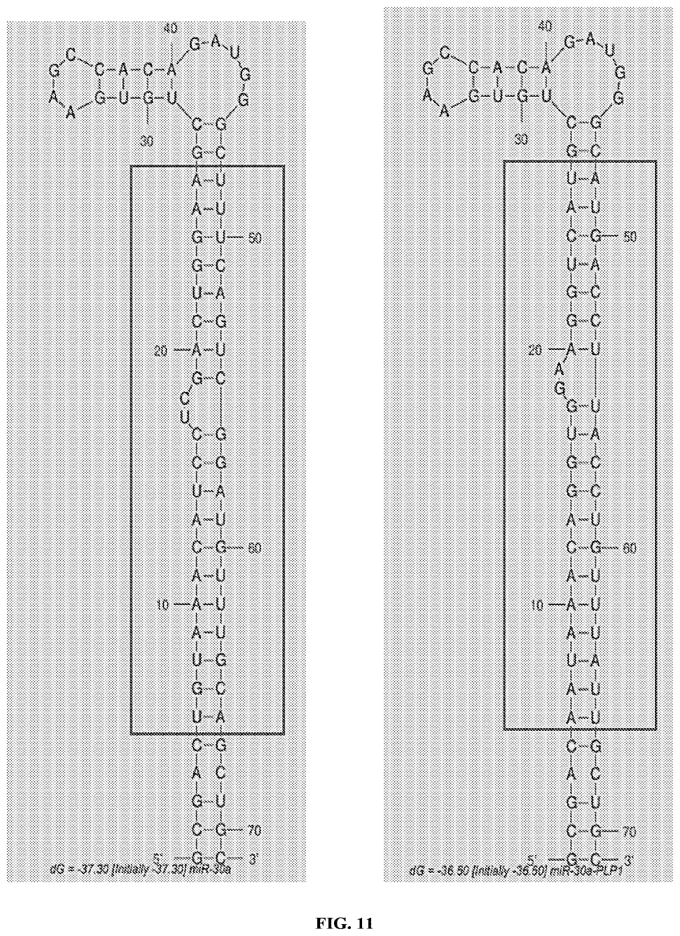

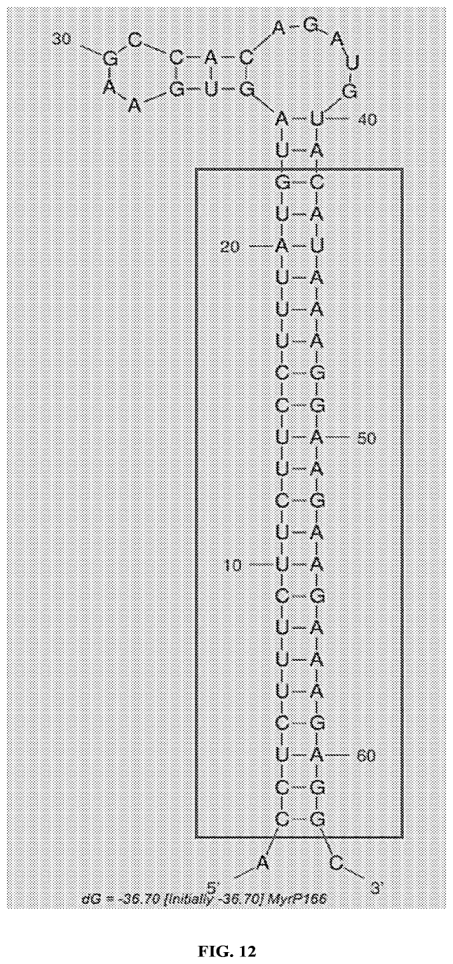

In some embodiments, the inhibitory nucleic acid is 5 to 300 bases in length (e.g., 10-30, 15-25, 19-22, 25-50, 40-90, 60-90, 75-100, 90-150, 110-200, 150-250, 200-300, etc. nucleotides in length). The inhibitory nucleic acid sequence encoding a pre-miRNA or mature miRNA may be 10-50, or 5-50 bases length. In some embodiments, the inhibitory nucleic acid encodes, or comprises, or consists essentially of, or consists of the sequence of SEQ ID NO: 284 or 285 as shown in FIG. 11 or 12.

### B. Scaffold

In certain embodiments, an inhibitory RNA molecule may be encoded in an inhibitory nucleic acid that comprises a molecular scaffold. As used herein a “molecular scaffold” is a framework or starting molecule that forms the sequence or structural basis against which to design or make a subsequent molecule.

In some embodiments, the molecular scaffold comprises at least one 5′ flanking, or one 3′ flanking region, or both. As a non-limiting example, the 5′ or 3′ flanking region may comprise a 5′ or 3′ flanking sequence which may be of any length and may be derived in whole or in part from wild type microRNA sequence or be a completely artificial sequence.

In some embodiments, one or both of the 5′ and 3′ flanking sequences may be absent. In some embodiments the 5′ and 3′ flanking sequences may be of the same or different length. In some embodiments the 5′ or 3′ flanking sequence may be from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In some embodiments, an inhibitory nucleic acid sequence comprising or encoding a pri-miRNA scaffold and is at least 200, 250, 260, 270, 280, 290, or 300 bases in length. In some embodiments, the inhibitory nucleic acid comprises or consists of a sequence of bases at least 80% or 90% complementary to, e.g., at least 5, 10, 15, 20, 25 or 30 bases of, or up to 30 or 40 bases of, a target nucleic acid (e.g., a human mRNA, such as that of PLP1, SNCA, or TUBB4A), or comprises a sequence of bases with up to 3 mismatches (e.g., up to 1, or up to 2 mismatches) over 10, 15, 20, 25 or 30 bases of the target nucleic acid.

In some embodiments, an inhibitory nucleic acid is an artificial miRNA (amiRNA). An amiRNA is derived by modifying a native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated.

Forming a stem of a stem loop structure is a minimum of the inhibitory nucleic acid encoding at least one siRNA, miRNA, shRNA or other RNAi agent described herein. In some embodiments, the siRNA, miRNA, shRNA, or other RNAi agent described herein comprises at least one nucleic acid sequence which is in part complementary or will hybridize to a target sequence. In some embodiments, the 5′ arm of the stem loop structure of the inhibitory nucleic acid comprises a nucleic acid sequence encoding an anti-sense sequence (i.e., a guide sequence/strand). In some other embodiments, the 3′ arm of the stem loop structure of the inhibitory nucleic acid comprises a nucleic acid sequence encoding the anti-sense/guide sequence.

In certain embodiments, separating the sense sequence and antisense sequence of the stem loop structure of the inhibitory nucleic acid is a loop sequence (also known as a loop motif, linker or linker motif). The loop sequence may be of any length, between 4-30 nucleotides, between 4-20 nucleotides, between 4-15 nucleotides, between 5-15 nucleotides, between 6-12 nucleotides, 6 nucleotides, 7 nucleotides, 8 nucleotides, 9 nucleotides, 10 nucleotides, 11 nucleotides, 12 nucleotides, 13 nucleotides, 14 nucleotides, and/or 15 nucleotides.

Some aspects of the disclosure relate to a nucleic acid sequence encoding a guide strand targeting a human gene that is inserted in a human or non-human (e.g., mouse) pri-miRNA scaffold. In some embodiments, a pri-miRNA scaffold can be selected from mir-16-1, miR-21, miR-23a, miRNA-30a, miR-31, miR-122, miR-155, or miR-451. In some embodiments, the pri-miRNA scaffold flanks an inhibitory nucleic acid targeting a human mRNA (such as that of PLP1, SNCA, or TUBB4A) or a target sequence thereof, (e.g., one encoded by those set forth in Tables 1-3 above, such as SEQ ID NO: 14 and SEQ ID NO: 35). In some embodiments, the pri-miRNA scaffold flanks an inhibitory nucleic acid comprising or encoding a guide strand (e.g., a guide strand RNA sequence corresponding to or encoded by one as set forth in Tables 1-3 above, such as SEQ ID NO: 54 and SEQ ID NO: 75). In some embodiments, the pri-miRNA scaffold can be from human miRNA-30a (FIG. 11, left). The related guide strand RNA sequence can be on either the 5′ arm or the 3′ arm of the stem loop. In one example, the guide strand RNA sequence corresponding to or encoded by SEQ ID NO: 75 can be on the 5′ arm (see FIG. 11, right). In another example, the guide strand RNA sequence corresponding to or encoded by SEQ ID NO: 54 can be on the 3′ arm (see FIG. 12).

### C. Recombinant Nucleic Acids

Recombinant nucleic acids of the present disclosure include inhibitory nucleic acids described above as well as plasmids and vector genomes that comprise an inhibitory nucleic acid. A recombinant nucleic acid, plasmid or vector genome may comprise regulatory sequences to modulate propagation (e.g., of a plasmid) and/or control expression of a transgene (e.g., an inhibitory nucleic acid). Recombinant nucleic acids may also be provided as a component of a viral vector (e.g., an rAAV vector). Generally, a viral vector includes a vector genome comprising a recombinant nucleic acid packaged in a capsid.

### D. Regulatory Elements

The present disclosure includes a recombinant nucleic acid including a transgene (e.g., one encoding an RNA) and various regulatory or control elements (e.g., a woodchuck hepatitis post-transcriptional regulatory element). Typically, regulatory elements are nucleic acid sequence(s) that influence expression of an operably linked polynucleotide. The precise nature of regulatory elements useful for gene expression will vary from organism to organism and from cell type to cell type including, for example, a promoter, enhancer, intron etc., with the intent to facilitate proper heterologous polynucleotide transcription and/or translation. Regulatory control can be affected at the level of transcription, translation, splicing, message stability, etc. Typically, a regulatory control element that modulates transcription is juxtaposed near the 5′ end of the transcribed polynucleotide (i.e., upstream). Regulatory control elements may also be located at the 3′ end of the transcribed sequence (i.e., downstream) or within the transcript (e.g., in an intron). Regulatory control elements can be located at a distance away from the transcribed sequence (e.g., 1 to 100, 100 to 500, 500 to 1000, 1000 to 5000, 5000 to 10,000 or more nucleotides). However, due to the length of a vector genome (e.g., an AAV vector genome), regulatory control elements are typically within 1 to 1000 nucleotides from the polynucleotide.

a. Promoter

As used herein, the term “promoter,” such as a “eukaryotic promoter,” refers to a nucleotide sequence that initiates transcription of a particular gene, or one or more coding sequences in eukaryotic cells (e.g., an oligodendrocyte). A promoter can work with other regulatory elements or regions to direct the level of transcription of the gene or coding sequence(s). These regulatory elements include, for example, transcription binding sites, repressor and activator protein binding sites, and other nucleotide sequences known to act directly or indirectly to regulate the amount of transcription from the promoter, including, for example, attenuators, enhances and silencers. The promoter is most often located on the same strand and near the transcription start site, 5′ of the gene or coding sequence to which it is operably linked. A promoter is generally 100-1000 nucleotides in length. A promoter typically increases gene expression relative to expression of the same gene in the absence of a promoter.

As used herein, a “core promoter” or “minimal promoter” refers to the minimal portion of a promoter sequence required to properly initiate transcription. It may include any of the following: a transcription start site, a binding site for RNA polymerase and a general transcription factor binding site. A promoter may also comprise a proximal promoter sequence (5′ of a core promoter) that contains other primary regulatory elements (e.g., enhancer, silencer, boundary element, insulator) as well as a distal promoter sequence (3′ of a core promoter).

Examples of suitable a promoter include adenoviral promoters, such as the adenoviral major late promoter; heterologous promoters, such as the cytomegalovirus (CMV) promoter; the respiratory syncytial virus promoter; the Rous Sarcoma Virus (RSV) promoter; the albumin promoter; inducible promoters, such as the Mouse Mammary Tumor Virus (MMTV) promoter; the metallothionein promoter; heat shock promoters; the α-1-antitrypsin promoter; the hepatitis B surface antigen promoter; the transferrin promoter; the apolipoprotein A-1 promoter; chicken 3-actin (CBA) promoter, the elongation factor 1a promoter (EF1a), the hybrid form of the CBA promoter (CBh promoter), and the CAG promoter (cytomegalovirus early enhancer element and the promoter, the first exon, and the first intron of chicken beta-actin gene and the splice acceptor of the rabbit beta-globin gene) (Alexopoulou et al. (2008) BioMed. Central Cell Biol. 9:2).

A promoter may be constitutive, tissue-specific or regulated. Constitutive promoters are those which cause an operably linked gene to be expressed at all times. In some embodiments, a constitutive promoter is active in most eukaryotic tissues under most physiological and developmental conditions.

Regulated promoters are those which can be activated or deactivated. Regulated promoters include inducible promoters, which are usually “off” but which may be induced to turn “on,” and “repressible” promoters, which are usually “on” but may be turned “off.” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

A tissue-specific promoter is a promoter that is active in only specific types of tissues, cells or organs. Typically, a tissue-specific promoter is recognized by transcriptional activator elements that are specific to a particular tissue, cell and/or organ. For example, a tissue-specific promoter may be more active in one or several particular tissues (e.g., two, three or four) than in other tissues. In some embodiments, expression of a gene modulated by a tissue-specific promoter is much higher in the tissue for which the promoter is specific than in other tissues. In some embodiments, there may be little, or substantially no activity, of the promoter in any tissue other than the one for which it is specific.

b. Enhancer

In another aspect, a recombinant nucleic acid described herein can further comprise an enhancer to increase expression of the transgene (e.g., a RNA molecule disclosed herein). Typically, an enhancer element is located upstream of a promoter element but may also be located downstream or within another sequence (e.g., a transgene). An enhancer may be located 100 nucleotides, 200 nucleotides, 300 nucleotides or more upstream or downstream of a modified nucleic acid. An enhancer typically increases expression of a transgene (e.g., encoding an inhibitory nucleic acid) beyond the increased expression provided by a promoter element alone.

Many enhancers are known in the art, including, but not limited to, the cytomegalovirus major immediate-early enhancer. More specifically, the CMV MIE promoter comprises three regions: the modulator, the unique region and the enhancer (Isomura and Stinski (2003) J. Virol. 77(6):3602-3614). The CMV enhancer region can be combined with another promoter, or a portion thereof, to form a hybrid promoter to further increase expression of a nucleic acid operably linked thereto. For example, a CBA promoter, or a portion thereof, can be combined with a CMV promoter/enhancer, or a portion thereof, to make a version of CBA termed the “CBh” promoter, which stands for chicken beta-actin hybrid promoter, as described in Gray et al. (2011, Human Gene Therapy 22:1143-1153). Like promoters, enhancers may be constitutive, tissue-specific or regulated.

c. Fillers, Spacers and Stuffers

As disclosed herein, a recombinant nucleic acid can be used in an rAAV vector. In that case, the recombinant nucleic acid may include an additional nucleic acid element to adjust the length of the nucleic acid to near, or at the normal size (e.g., approximately 4.7 to 4.9 kilobases), of the viral genomic sequence acceptable for AAV packaging into an rAAV vector (Grieger and Samulski (2005) J. Virol. 79(15):9933-9944). Such a sequence may be referred to interchangeably as filler, spacer or stuffer. In some embodiments, filler DNA is an untranslated (non-protein coding) segment of nucleic acid. In some embodiments, a filler or stuffer polynucleotide sequence is a sequence between about 1-10, 10-20, 20-30, 30-40, 40-50, 50-60, 60-70, 70-80, 80-90-90-100, 100-150, 150-200, 200-250, 250-300, 300-400, 400-500, 500-750, 750-1000, 1000-1500, 1500-2000, 2000-3000 or more in length.

AAV vectors typically accept inserts of DNA having a size ranging from about 4 kb to about 5.2 kb or about 4.1 to 4.9 kb for optimal packaging of the nucleic acid into the AAV capsid. In some embodiments, an rAAV vector comprises a vector genome having a total length between about 3.0 kb to about 3.5 kb, about 3.5 kb to about 4.0 kb, about 4.0 kb to about 4.5 kb, about 4.5 kb to about 5.0 kb or about 5.0 kb to about 5.2 kb. In some embodiments, an rAAV vector comprises a vector genome having a total length of about 4.7 kb. In some embodiments, an rAAV vector comprises a vector genome that is self-complementary. While the total length of a self-complementary (sc) vector genome in an rAAV vector is equivalent to a single-stranded (ss) vector genome (i.e., from about 4 kb to about 5.2 kb), the nucleic acid sequence (i.e., comprising the transgene, regulatory elements and ITRs) encoding the sc vector genome must be only half as long as a nucleic acid sequence encoding a ss vector genome in order for the sc vector genome to be packaged in the capsid.

d. Introns and Exons

In some embodiments, a recombinant nucleic acid disclosed herein includes, for example, an intron, exon and/or a portion thereof. An intron may function as a filler or stuffer polynucleotide sequence to achieve an appropriate length for vector genome packaging into an rAAV vector. An intron and/or an exon sequence can also enhance expression of a transgene (e.g., an RNA disclosed herein) as compared to expression in the absence of the intron and/or exon element (Kurachi et al. (1995) J. Biol. Chem. 270 (10):576-5281; WO 2017/074526). Furthermore, filler/stuffer polynucleotide sequences (also referred to as “insulators”) are well known in the art and include, but are not limited to, those described in WO 2014/144486 and WO 2017/074526.

e. Polyadenylation Signal Sequence (polyA)

Further regulatory elements may include a stop codon, a termination sequence, and a polyadenylation (polyA) signal sequence, such as, but not limited to a bovine growth hormone poly A signal sequence (BHG polyA). A polyA signal sequence drives efficient addition of a poly-adenosine “tail” at the 3′ end of a eukaryotic mRNA which guides termination of gene transcription (see, e.g., Goodwin and Rottman J. Biol. Chem. (1992) 267(23):16330-16334). A polyA signal acts as a signal for the endonucleolytic cleavage of the newly formed precursor mRNA at its 3′ end and for addition to this 3′ end of an RNA stretch consisting only of adenine bases. A polyA tail is important for the nuclear export, translation and stability of mRNA. In some embodiments, a poly A can be a SV40 early polyadenylation signal, a SV40 late polyadenylation signal, an HSV thymidine kinase polyadenylation signal, a protamine gene polyadenylation signal, an adenovirus 5 E1b polyadenylation signal, a growth hormone polyadenylation signal, a PBGD polyadenylation signal or an in silico designed polyadenylation signal.

### 3. Expression Cassettes and Expression Vectors

The disclosure also provides an expression cassette, comprising or consisting of a recombinant nucleic acid encoding an inhibitory nucleic acid as described above. Where such recombinant nucleic acid may not already comprise a promoter, the expression cassette may additionally comprise a promoter. Thus, an expression cassette according to the present invention comprises, in 5′ to 3′ direction, a promoter, a coding sequence, and optionally a terminator or other elements. The expression cassette allows an easy transfer of a nucleic acid sequence of interest into an organism, preferably a cell and preferably a disease cell.

The expression cassette of the present disclosure is preferably comprised in a vector. Thus, the vector of the present disclosure allows to transform a cell with a nucleic acid sequence of interest. Correspondingly the disclosure provides a host cell comprising an expression cassette according to the present disclosure or a recombinant nucleic acid according to the present disclosure. The recombinant nucleic acid may also comprise a promoter or enhancer such as to allow for the expression of the nucleic acid sequence of interest.

Exogenous genetic material (e.g., a nucleic acid, an expression cassette, or an expression vector encoding one or more therapeutic or inhibitory RNAs) can be introduced into a target cells of interest in vivo by genetic transfer methods, such as transfection or transduction, to provide a genetically modified cell. Various expression vectors (i.e., vehicles for facilitating delivery of exogenous genetic material into a target cell) are known to one of ordinary skill in the art. As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into an RNA.

As used herein, “transfection of cells” refers to the acquisition by a cell of new genetic material by incorporation of added nucleic acid (DNA, RNA, or a hybrid thereof) without use of a viral delivery vehicle. Thus, transfection refers to the introducing of nucleic acid into a cell using physical or chemical methods. Several transfection techniques are known to those of ordinary skill in the art including: calcium phosphate nucleic acid co-precipitation, strontium phosphate nucleic acid co-precipitation, DEAE-dextran, electroporation, cationic liposome-mediated transfection, and tungsten particle-facilitated microparticle bombardment. In contrast, “transduction of cells” refers to the process of transferring nucleic acid into a cell using a DNA or RNA virus. An RNA virus (e.g., a retrovirus) for transferring a nucleic acid into a cell is referred to herein as a transducing chimeric virus. Exogenous genetic material contained within the virus can be incorporated into the genome of the transduced cell. A cell that has been transduced with a chimeric DNA virus (e.g., an adenovirus carrying a DNA encoding a therapeutic agent), may not have the exogenous genetic material incorporated into its genome but may be capable of expressing the exogenous genetic material that is retained extrachromosomally within the cell.

Typically, the exogenous genetic material may include a heterologous gene (coding for a therapeutic RNA or protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase, dihydrofolate reductase, adenosine deaminase, phosphoglycerol kinase, pyruvate kinase, phosphoglycerol mutase, the actin promoter, ubiquitin, elongation factor-1 and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only in, or largely controlled by, the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene or a fluorescent protein gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene, and/or signal sequence is deemed to be within the scope of one of ordinary skill in the art without undue experimentation.

A coding sequence of the present disclosure can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

As disclosed herein, the RNA molecules described above can be used for treating a disorder in a subject. In some embodiments, a polynucleotide encoding the RNA molecule can be inserted into, or encoded by, vectors such as plasmids or viral vectors. Preferably, the polynucleotide is inserted into, or encoded by, viral vectors. Viral vectors may be Herpesvirus (HSV) vectors, retroviral vectors, adenoviral vectors, AAV vectors, lentiviral vectors, and the like. In some specific embodiments, the viral vectors are AAV vectors. In some embodiments, the RNA may be encoded by a retroviral vector (See, e.g., U.S. Pat. Nos. 5,399,346; 5,124,263; 4,650,764 and 4,980,289; the content of each of which is incorporated herein by reference in their entirety).

Adenoviruses are eukaryotic DNA viruses that can be modified to efficiently deliver a nucleic acid to a variety of cell types in vivo, and have been used extensively in gene therapy protocols, including for targeting genes to neural cells and glial cells. Various replication defective adenovirus and minimum adenovirus vectors have been described for nucleic acid therapeutics (See, e.g., PCT Patent Publication Nos. WO199426914, WO 199502697, WO199428152, WO199412649, WO199502697 and WO199622378; the content of each of which is incorporated by reference in their entirety). Such adenoviral vectors may also be used to deliver RNA molecules of the present disclosure to cells.

### 4. AAV

The adeno-associated virus is a widely used gene therapy vector due to its clinical safety record, non-pathogenic nature, ability to infect non-dividing cells (like neurons), and ability to provide long-term gene expression after a single administration (Hocquemiller et al., 2016). Currently, many human and non-human primate AAV serotypes have been identified (Gao et al., 2004). AAV vectors have demonstrated safety in hundreds of clinical trials worldwide, and clinical efficacy has been shown in trials of hemophilia B, spinal muscular atrophy, alpha 1 antitrypson, and Leber congenital amaurosis (Keeler et al., 2017). Three AAV-based gene therapies have been approved. The first, Glybera, was approved by the European Medicines Agency (EMA) in 2012 (though withdrawn in 2017 mainly due to commercial failure). Luxturna was approved by FDA in 2017 for a rare inherited retinal dystrophy, and Zolgensma was approved by FDA in 2019 for spinal muscular atrophy.

Because of their safety, nonpathogenic nature, and ability to infect neurons, AAVs such as AAV1, AAV2, AAV4, AAV5, AAV6, AAV8, and AAV9 are commonly used gene therapy vectors for CNS applications. However, after direct CNS infusion, these serotypes exhibit a dominant neuronal tropism and expression in oligodendrocytes is low, especially when gene expression is driven by a constitutive promoter, which restricts their potential for use in treating white matter diseases. AAV1/2, AAV2, and AAV8 have been shown transduce oligodendrocytes, but only when oligodendrocyte-specific promoters are used (Chen et al., 1998; Lawlor et al., 2009; Li et al., 2019). Reliance on cell-specific promoters for expression specificity allows for the possibility of nonselective cellular uptake and leaky transgene expression through cryptic promoter activity in non-oligodendrocyte lineage cells.

The approach described herein to alleviate these issues includes using AAV serotypes with high tropism for oligodendrocytes. Recently, using DNA shuffling and directed evolution, a chimeric AAV capsid with strong selectivity for oligodendrocytes, AAV/Olig001, has been described (Powell et al., 2016). Subsequently, AAV/Olig001 was shown to transduce neonatal oligodendrocytes in a mouse model of Canavan disease (Francis et al., 2021). Other approaches such as random mutagenesis and peptide library insertion can be used to generate capsid libraries that can be screened for tropism and selectivity for oligodendrocytes.

As discussed above, the terms “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, RNAs or polypeptides encoded by the nucleic acid can be expressed by the cell. Because AAV is not associated with any pathogenic disease in humans, a nucleic acid delivered by AAV can be used to express a therapeutic RNA or polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. Examples include AAV1, AAV2, AAV, AAV3 (including AAV3A and AAV3B), AAV4, AAV5, AAV6, AAV7, AAV8, AAV9, AAV10, AAV12, AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV2i8, NP4, NP22, NP66, DJ, DJ/8, DJ/9, LK3, RHM4-1, among many others. “Primate AAV” refers to AAV that infect primates, “non-primate AAV” refers to AAV that infect non-primate mammals, “bovine AAV” refers to AAV that infect bovine mammals, and so on.

Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, as well as viruses that are not serologically distinct but that may be within a subgroup or a variant of a given serotype.

A comprehensive list and alignment of amino acid sequences of capsids of known AAV serotypes is provided by Marsic et al. (2014) Molecular Therapy 22(11):1900-1909. Genomic sequences of various serotypes of AAV, as well as sequences of the native ITRs, rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001863 (AAV3B), NC_001829 (AAV4), U89790 (AAV4), NC_006152 (AAV5), NC_001862 (AAV6), AF513851 (AAV7), AF513852 (AAV8), and NC_006261 (AAV8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111.

As discussed herein, a “recombinant adeno-associated virus” or “rAAV” is distinguished from a wild-type AAV by replacement of all or part of the endogenous viral genome with a non-native sequence. Incorporation of a non-native sequence within the virus defines the viral vector as a “recombinant” vector, and hence a “rAAV vector.” An rAAV vector can include a heterologous polynucleotide encoding a desired RNA or protein or polypeptide (e.g., an RNA molecule disclosed herein). A recombinant vector sequence may be encapsidated or packaged into an AAV capsid and referred to as an “rAAV vector,” an “rAAV vector particle,” “rAAV viral particle” or simply a “rAAV.”

For the production of an rAAV vector, the desired ratio of VP1:VP2:VP3 can be in the range of about 1:1:1 to about 1:1:100, preferably in the range of about 1:1:2 to about 1:1:50, more preferably in the range of about 1:1:5 to about 1:1:20. Although the desired ratio of VP1:VP2 can be 1:1, the ratio range of VP1:VP2 could vary from 1:50 to 50:1.

The present disclosure provides for an rAAV vector comprising a polynucleotide sequence not of AAV origin (e.g., a polynucleotide heterologous to AAV). The heterologous polynucleotide may be flanked by at least one, and sometimes by two, AAV terminal repeat sequences (e.g., inverted terminal repeats). The heterologous polynucleotide flanked by ITRs, also referred to herein as a “vector genome,” typically encodes an RNA or a polypeptide of interest, or a gene of interest, such as a target for therapeutic treatment. Delivery or administration of an rAAV vector to a subject (e.g. a patient) provides encoded RNAs/proteins/peptides to the subject. Thus, an rAAV vector can be used to transfer/deliver a heterologous polynucleotide for expression for, e.g., treating a variety of diseases, disorders and conditions.

rAAV vector genomes generally retain 145 base ITRs in cis to the heterologous nucleic acid sequence that replaced the viral rep and cap genes. Such ITRs are useful to produce a recombinant AAV vector; however, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. An rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV.

An rAAV vector of the disclosure may comprise an ITR from an AAV serotype (e.g., wild-type AAV2, a fragment or variant thereof) that differs from the serotype of the capsid (e.g., AAV8, Olig001). Such an rAAV vector comprising at least one ITR from one serotype, but comprising a capsid from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, an rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from an rAAV vector can be hindered by the need to convert a single stranded rAAV genome (ssAAV) into double-stranded DNA prior to expression. This step can be circumvented by using a self-complementary AAV genome (scAAV) that can package an inverted repeat genome that can fold into double-stranded DNA without the need for DNA synthesis or base-pairing between multiple vector genomes. See, e.g., U.S. Pat. No. 8,784,799; McCarty, (2008) Molec. Therapy 16(10):1648-1656; and McCarty et al., (2001) Gene Therapy 8:1248-1254; McCarty et al., (2003) Gene Therapy 10:2112-2118.

A viral capsid of an rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4^{th} ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In some embodiments, an rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or subunit can be replaced by a B19 cap protein or subunit. For example, in one embodiment, a VP3 subunit of an AAV capsid can be replaced by a VP2 subunit of B19. In some embodiments, a chimeric capsid is an Olig001 capsid as described in WO2021221995 and WO2014052789, which are incorporated herein by reference.

In some embodiments, chimeric vectors have been engineered to exhibit altered tropism or tropism for a particular tissue or cell type. The term “tropism” refers to preferential entry of the virus into certain cell (e.g., oligodendrocytes) or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., an rAAV vector genome) are expressed.

A “tropism profile” refers to a pattern of transduction of one or more target cells in various tissues and/or organs. For example, a chimeric AAV capsid may have a tropism profile characterized by efficient transduction of oligodendrocytes with only low transduction of neurons, astrocytes and other CNS cells. See WO2014/052789, incorporated herein by reference. Such a chimeric capsid may be considered “specific for oligodendrocytes” exhibiting tropism for oligodendrocytes, and referred to herein as “oligotropism,” if when administered directly into the CNS, preferentially transduces oligodendrocytes over neurons, astrocytes and other CNS cell types. In some embodiments, at least about 80% of cells that are transduced by a capsid specific for oligodendrocytes are oligodendrocytes, e.g., at least about 85%, 90%, 95%, 96%, 97%, 98% 99% or more of the transduced cells are oligodendrocytes.

In some embodiments, an rAAV vector is useful for treating or preventing a “disorder associated with oligodendrocyte dysfunction.” As used herein, the term “associated with oligodendrocyte dysfunction” refers to a disease, disorder or condition in which oligodendrocytes are damaged, lost or function improperly compared to otherwise identical normal oligodendrocytes. The term includes diseases, disorders and conditions in which oligodendrocytes are directly affected as well as diseases, disorders or conditions in which oligodendrocytes become dysfunctional secondary to damage to other cells. In some embodiments, a disorder associated with oligodendrocyte dysfunction is demyelination.

In some embodiments, a chimeric AAV capsid with tropism for oligodendrocytes is Olig001 (also known as BNP61) or a functional variant of Olig001, which comprises sequences from AAV1, AAV2, AAV6, AAV8 and AAV9 (see WO2021221995 and WO 2014/052789). The amino acid sequence of the Olig001 capsid protein is set forth in the sequence below with VP1 starting at amino acid residue 1 (methionine), VP2 starting at amino acid residue 148 (threonine) and VP3 starting at amino acid residue 203 (methionine).

Amino acid sequence for Olig001 (BNP61) capsid
(SEQ ID NO: 245)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRLLEPLGLVEEAAKTAPGKKRPVE
QSPQEPDSSSGIGKTGQQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNGTSGGATNDNTYFGYSTPWGYFDFNRFHCHFS
PRDWQRLINNNWGFRPKRLSFKLFNIQVKEVTQNEGTKTIANNLTSTVQ
VFTDSEYQLPYVLGSAHQGCLPPFPADVFMIPQYGYLTLNNGSQAVGRS
SFYCLEYFPSQMLRTGNNFQFTYTFEDVPFHSSYAHSQSLDRLMNPLID
QYLYYLSRTQTTGGTANTQTLGFSQGGPNTMANQAKNWLPGPCYRQQRV
STTTGQNNNSNFAWTAGTKYHLNGRNSLANPGIAMATHKDDKERFFPSN
GILIFGKQNAARDNADYSDVMLTSEEEIKTTNPVATEEYGIVADNLQQQ
NTAPQIGTVNSQGALPGMVWQNRDVYLQGPIWAKIPHTDGNFHPSPLMG
GFGLKHPPPQILIKNTPVPADPPTTFNQSKINSFITQYSTGQVSVEIEW
ELQKENSKRWNPEIQYTSNYYKSTSVDFAVNTEGVYSEPHPIGTRYLTR
PL

In some embodiments, a chimeric AAV capsid with tropism for oligodendrocytes is Olig002 (also known as BNP62) or Olig003 (also known as BNP63) (see WO2021221995 and WO 2014/052789). In some embodiments, the Olig002 capsid VP1 comprises or consists of the amino acid sequence shown below or a functional variant thereof.

Amino acid sequence for Olig002 (BNP62) capsid
(SEQ ID NO: 246)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAVFQAKKRVLEPLGLVEEGAKTAPGKKRPVE
QSPQEPDSSSGIGKTGQQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISSASTGASNDNHYFGYSTPWGYFDFNRFHCHFSP
RDWQRLINNNWGFRPKRLNFKLFNIQVKEVTDNNGVKTIANNLTSTVQV
FTDSDYQLPYVLGSAHQGCLPPFPADVFMIPQYGYLTLNNGSQAVGRSS
FYCLEYFPSQMLRTGNNFQFTYTFEDVPFHSSYAHSQSLDRLMNPLIDQ
YLYYLSRTQTTGGTANTQTLGFSQGGPNTMANQAKNWLPGPCYRQQRVS
TTTGQNNNSNFAWTAGTKYHLNGRNSLANPGIAMATHKDDKERFFPSNG
ILIFGKQNAARDNADYSDVMLTSEEEIKTTNPVATEEYGIVADNLQQQN
TAPQIGTVNSQGALPGMVWQNRDVYLQGPIWAKIPHTDGNFHPSPLMGG
FGLKHPPPQILIKNTPVPADPPTTFNQSKLNSFITQYSTGQVSVEIEWE
LQKENSKRWNPEIQYTSNYYKSTSVDFAVNTEGVYSEPHPIGTRYLTRP
L

In some embodiments, the Olig003 capsid comprises or consists of the amino acid sequence the amino acid sequence shown below or a functional variant thereof.

Amino acid sequence for Olig003 (BNP63) capsid
(SEQ ID NO: 247)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLQGDTSFGGNLGRAVFQAKKRVLEPLGLVEEGAKTAPGKKRPVE
QSPQEPDSSSGIGETGQQPAKKRLNFGQTGDSESVPDPQPLGEPPATPA
AVGPTTMASGGGAPMADNNEGADGVGSSSGNWHCDSQWLGDRVITTSTR
TWALPTYNNHLYKQISSASTGASNDNHYFGYSTPWGYFDFNRFHCHFSP
RDWQRLINNNWGFRPKRLSFKLFNIQVKEVTDNNGVKTIANNLTSTVQV
FTDSEYQLPYVLGSAHQGCLPPFPADVFMIPQYGYLTLNNGSQAVGRSS
FYCLEYFPSQMLRTGNNFTFSYTFEDVPFHSSYAHSQSLDRLMNPLIDQ
YLYYLSRTQTTGGTANTQTLGFSQGGPNTMANQAKNWLPGPCYRQQRVS
TTTGQNNNSNFAWTAGTKYHLNGRNSLANPGIAMATHKDDKERFFPSNG
ILIFGKQNAARDNADYSDVMLTSEEEIKTTNPVATEEYGIVADNLQQQN
TAPQIGTVNSQGALPGMVWQNRDVYLQGPIWAKIPHTDGNFHPSPLMGG
FGLKHPPPQILIKNTPVPADPPTTFNQSKLNSFITQYSTGQVSVEIEWE
LQKENSKRWNPEIQYTSNYYKSTSVDFAVNTEGVYSEPHPIGTRYLTRP
L

In some embodiments, an rAAV vector comprising a chimeric AAV capsid (e.g., Olig001, Olig002, or Olig003) and a therapeutic transgene may be used to treat a disease, disorder or condition associated with oligodendrocyte dysfunction. In such a disease, disorder or condition, oligodendrocytes are damaged, lost or function improperly. This may be the result of a direct effect on the oligodendrocyte or result when oligodendrocytes become dysfunctional secondary to damage to other cells. In some embodiments, an rAAV vector comprising an AAV/Olig001 capsid and a polynucleotide encoding a RNA molecule described herein is used to treat an inherited and acquired disorder of myelin.

### 5. Viral Particles and Productions

A viral vector (e.g., rAAV vector) carrying a transgene (e.g., one encoding an RNA disclosed herein) can be assembled from a polynucleotide encoding a transgene, suitable regulatory elements and elements necessary for production of viral proteins which mediate cell transduction. Examples of a viral vector include but are not limited to adenoviral, retroviral, lentiviral, herpesvirus and AAV vectors, and in particular rAAV vector.

A vector genome component of an rAAV vector produced according to the methods of the disclosure include at least one transgene (e.g., a polynucleotide encoding the RNA molecule) and associated expression control sequences for controlling expression of the RNA. In a preferred embodiment, a vector genome includes a portion of a parvovirus genome, such as an AAV genome with rep and cap deleted and/or replaced by a transgene and its associated expression control sequences. The transgene is typically inserted adjacent to one or two (i.e., is flanked by) AAV ITRs or ITR elements adequate for viral replication, in place of the nucleic acid encoding viral rep and cap proteins. Other regulatory sequences suitable for use in facilitating tissue-specific expression of the transgene in the target cell (e.g., oligodendrocyte) may also be included.

### A. Packaging Cell

One skilled in the art would appreciate that an rAAV vector comprising a transgene, and lacking virus proteins needed for viral replication (e.g., cap and rep), cannot replicate since such proteins are necessary for virus replication and packaging. Cap and rep genes may be supplied to a cell (e.g., a host cell, e.g., a packaging cell) as part of a plasmid that is separate from a plasmid supplying the vector genome with the transgene.

Packaging cell or producer cell means a cell or cell line which may be transfected with a vector, plasmid or DNA construct, and provides in trans all the missing functions which are required for the complete replication and packaging of a viral vector. The required genes for rAAV vector assembly include the vector genome (e.g., a transgene encoding an RNA, regulatory elements, and ITRs), AAV rep gene, AAV cap gene, and certain helper genes from other viruses such as, e.g., adenovirus. One of ordinary skill would understand that the requisite genes for AAV production can be introduced into a packaging cell in various ways including, for example, transfection of one or more plasmids. However, in some embodiments, some genes (e.g., rep, cap, helper) may already be present in a packaging cell, either integrated into the genome or carried on an episome. In some embodiments, a packaging cell expresses, in a constitutive or inducible manner, one or more missing viral functions.

Any suitable packaging cell known in the art may be employed in the production of a packaged viral vector. Mammalian cells or insect cells are preferred. Examples of cells useful for the production of a packaging cell in the practice of the disclosure include, for example, human cell lines, such as PER.C6, WI38, MRC5, A549, HEK293 cells (which express functional adenoviral E1 under the control of a constitutive promoter), B-50 or any other HeLa cell, HepG2, Saos-2, HuH7, and HT1080 cell lines. Suitable non-human mammalian cell lines include, for example, VERO, COS-1, COS-7, MDCK, BHK21-F, HKCC or CHO cells.

In some embodiments, a packaging cell is capable of growing in suspension culture. In some embodiments, a packaging cell is capable of growing in serum-free media. For example, HEK293 cells are grow in suspension in serum free medium. In another embodiment, a packaging cell is a HEK293 cell as described in U.S. Pat. No. 9,441,206 and deposited as American Type Culture Collection (ATCC) No. PTA 13274. Numerous rAAV packaging cell lines are known in the art, including, but not limited to, those disclosed in WO 2002/46359.

A cell line for use as a packaging cell includes insect cell lines. Any insect cell which allows for replication of AAV and which can be maintained in culture can be used in accordance with the present disclosure. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, NJ (1995); O'Reilly et al., Baculovirus Expression Vectors: A Laboratory Manual, Oxford Univ. Press (1994); Samulski et al. (1989) J. Virol. 63:3822-3828; Kajigaya et al. (1991) Proc. Nat'l. Acad. Sci. USA 88: 4646-4650; Ruffing et al. (1992) J. Virol. 66:6922-6930; Kimbauer et al. (1996) Virol. 219:37-44; Zhao et al. (2000) Virol. 272:382-393; and U.S. Pat. No. 6,204,059.

As a further alternative, viral vectors of the disclosure may be produced in insect cells using baculovirus vectors to deliver the rep/cap genes and rAAV template as described, for example, by Urabe et al. (2002) Human Gene Therapy 13:1935-1943. When using baculovirus production for AAV, in some embodiments, a vector genome is self-complementary. In some embodiments, a host cell is a baculovirus-infected cell (e.g., an insect cell) comprising, optionally, additional nucleic acids encoding baculovirus helper functions, thereby facilitating production of a viral capsid.

A packaging cell generally includes one or more viral vector functions along with helper functions and packaging functions sufficient to result in replication and packaging of the viral vector. These various functions may be supplied together, or separately, to the packaging cell using a genetic construct such as a plasmid or an amplicon, and they may exist extrachromosomally within the cell line, or integrated into the host cell's chromosomes.

### B. Helper Function

AAV cannot replicate in a cell without co-infection of the cell by a helper virus. Helper functions include helper virus elements needed for establishing active infection of a packaging cell, which is required to initiate packaging of the viral vector. Helper viruses include, typically, adenovirus or herpes simplex virus. Adenovirus helper functions typically include adenovirus components adenovirus early region 1A (E1a), E1b, E2a, E4, and viral associated (VA) RNA. Helper functions (e.g., E1a, E1b, E2a, E4, and VA RNA) can be provided to a packaging cell by transfecting the cell with one or more nucleic acids encoding various helper elements. Alternatively, a host cell (e.g., a packaging cell) can comprise a nucleic acid encoding the helper protein. For instance, HEK293 cells were generated by transforming human cells with adenovirus 5 DNA and now express a number of adenoviral genes, including, but not limited to E1 and E3 (see, e.g., Graham et al. (1977) J. Gen. Virol. 36:59-72). Thus, those helper functions can be provided by the HEK 293 packaging cell without the need of supplying them to the cell by, e.g., a plasmid encoding them.

In some embodiments, a packaging cell is transfected with at least (i) a plasmid comprising a vector genome comprising a transgene and AAV ITRs and further comprising at least one of the following regulatory elements: an enhancer, a promoter, an exon, an intron, and a poly A, (ii) a plasmid comprising a rep gene (e.g., AAV2 rep) and a cap gene (e.g., Olig001 cap) and (iii) a plasmid comprising a helper function.

Any method of introducing a nucleotide sequence carrying a helper function into a cellular host for replication and packaging may be employed, including but not limited to, electroporation, calcium phosphate precipitation, microinjection, cationic or anionic liposomes, and liposomes in combination with a nuclear localization signal. In some embodiments, helper functions are provided by transfection using a virus vector, or by infection using a helper virus, standard methods for producing viral infection may be used.

The vector genome may be any suitable recombinant nucleic acid, such as a DNA or RNA construct and may be single stranded, double stranded, or duplexed (i.e., self-complementary as described in WO 2001/92551).

### C. Production of Packaged Viral Vector

Viral vectors can be made by several methods known to skilled artisans (see, e.g., WO 2013/063379). A preferred method is described in Grieger, et al. (2015) Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using a triple transfection method (e.g., WO 96/40240), a HEK293 cell line suspension can generate greater than 1×10⁵ vector genome containing particles (vg)/cell, or greater than 1×10¹⁴ vg/L of cell culture, when harvested 48 hours post-transfection. More specifically, triple transfection refers a method whereby a packaging cell is transfected with three plasmids: one plasmid encodes the AAV rep and cap (e.g., Olig001 cap) genes, another plasmid encodes various helper functions (e.g., adenovirus or HSV proteins such as E1a, E1b, E2a, E4, and VA RNA, and another plasmid encodes a transgene (e.g., an RNA described herein) and various elements to control expression of the transgene.

Single-stranded vector genomes are packaged into capsids as the plus strand or minus strand in about equal proportions. In some embodiments of an rAAV vector, a vector genome is in the plus strand polarity (i.e., the sense or coding sequence of the DNA strand). In some embodiments an rAAV vector, a vector is in the minus strand polarity (i.e., the antisense or template DNA strand). Given the nucleotide sequence of a plus strand in its 5′ to 3′ orientation, the nucleotide sequence of a minus strand in its 5′ to 3′ orientation can be determined as the reverse-complement of the nucleotide sequence of the plus strand.

To achieve the desired yields, a number of variables are optimized such as selection of a compatible serum-free suspension media that supports both growth and transfection, selection of a transfection reagent, transfection conditions and cell density.

An rAAV vector may be purified by methods standard in the art such as by column chromatography or cesium chloride gradients. Methods for purifying rAAV vectors are known in the art and include methods described in Clark et al. (1999) Human Gene Therapy 10(6):1031-1039; Schenpp and Clark (2002) Methods Mol. Med. 69:427-443; U.S. Pat. No. 6,566,118 and WO 98/09657.

A universal purification strategy, based on ion exchange chromatography methods, may be used to generate high purity vector preps of AAV serotypes 1-6, 8, 9 and various chimeric capsids (e.g., Olig001). In some embodiment, this process can be completed within one week, result in high full to empty capsid ratios (>90% full capsids), provide post-purification yields (>1×10¹³ vg/L) and purity suitable for clinical applications. In some embodiments, such a method is universal with respect to all serotypes and chimeric capsids. Scalable manufacturing technology may be utilized to manufacture GMP clinical and commercial grade rAAV vectors (e.g., for the treatment of an inherited or acquired disorder of myelin).

After rAAV vectors of the present disclosure have been produced and purified, they can be titered (e.g., the amount of rAAV vector in a sample can be quantified) to prepare compositions for administration to subjects, such as human subjects with an inherited or acquired disorder of myelin. rAAV vector titering can be accomplished using methods know in the art.

In some embodiments, the number of viral particles, including particles containing a vector genome and “empty” capsids that do not contain a vector genome, can be determined by electron microscopy, e.g., transmission electron microscopy (TEM). Such a TEM-based method can provide the number of vector particles (or virus particles in the case of wild type AAV) in a sample.

In some embodiments, rAAV vector genomes can be titered using quantitative PCR (qPCR) using primers against sequences in the vector genome, for example ITR sequences, and/or sequences in the transgene or regulatory elements. By performing qPCR in parallel on dilutions of a standard of known concentration, such as a plasmid containing the sequence of the vector genome, a standard curve can be generated permitting the concentration of the rAAV vector to be calculated as the number of vector genomes (vg) per unit volume such as microliters or milliliters. By comparing the number of vector particles as measured by, e.g., electron microscopy, to the number of vector genomes in a sample, the number of empty capsids can be determined. Because the vector genome contains the therapeutic transgene, vg/kg or vg/ml of a vector sample may be more indicative of the therapeutic amount of the vector that a subject will receive than the number of vector particles, some of which may be empty and not contain a vector genome. Once the concentration of rAAV vector genomes in the stock solution is determined, it can be diluted into or dialyzed against suitable buffers for use in preparing a composition for administration to subjects (e.g., subjects with an inherited or acquired disorder of myelin).

### 6. Uses and Treatment Methods

A nucleic acid (such as an RNA molecule or polynucleotide encoding the RNA molecule) as disclosed herein may be used for gene therapy treatment and/or prevention of a disease, disorder or condition. In particular, it can be used for treating or preventing a disease, disorder or condition associated with deficiency or dysfunction of oligodendrocyte or myelin by targeting a particular target gene (e.g., PMD. MSA, or H-ABC), and of any other condition and or illness in which reducing the expression of the related target gene may produce a therapeutic benefit or improvement, e.g., a disease, disorder or condition mediated by, or associated with, an increase in the level or function of the related protein (e.g., PLP1, SNCA, or TUBB4A) compared with the level or function of the protein in an otherwise healthy individual. The vector genome and/or an rAAV vector described herein can be used for gene therapy treatment and/or prevention of the same disease, disorder or condition.

In some embodiments, methods of the disclosure include use of an rAAV vector, or a pharmaceutical composition thereof, in the treatment of the disease, disorder or condition in a subject. In some embodiments, methods of the disclosure include use of an rAAV vector (e.g., AAV/Olig001), or pharmaceutical composition thereof, to decrease the level of a gene of interest (e.g., PMD. MSA, or H-ABC) in a subject in need thereof.

The nucleic acid, a vector genome, and/or an rAAV vector described above can be used in the preparation of a medicament for use in the treatment and/or prevention of a disease, disorder or condition associated with or caused by deficiency or dysfunction of oligodendrocyte or myelin (e.g., PMD. MSA, or H-ABC) and of any other condition or illness in which down-regulation of the related protein(s) may produce a therapeutic benefit or improvement.

As used herein a disorder of myelin, a disease of myelin, a myelin-related disorder, a myelin-related disease, a myelin disorder, and a myelin disease are used interchangeably. They include any disease, condition (e.g., those occurring from traumatic spinal cord injury and cerebral infarction), or disorder related to demyelination, insufficient myelination and remyelination, or dysmyelination in a subject. Such a disorder can be inherited or acquired or both. It can arise from a myelination related disorder or demyelination resulting from a variety of neurotoxic insults. “Demyelination” as used herein, refers to the act of demyelinating, or the loss of the myelin sheath insulating the nerves, and is the hallmark of some neurodegenerative autoimmune diseases, including multiple sclerosis, transverse myelitis, chronic inflammatory demyelinating polyneuropathy, and Guillain-Barre Syndrome. Leukodystrophies are caused by inherited enzyme deficiencies, which cause abnormal formation, destruction, and/or abnormal turnover of myelin sheaths within the CNS white matter. Both acquired and inherited myelin disorders share a poor prognosis leading to major disability. Thus, some embodiments of the present disclosure can include methods for the treatment of neurodegenerative autoimmune diseases in a subject. Remyelination of neurons requires oligodendrocytes. The term “remyelination”, as used herein, refers to the re-generation of the nerve's myelin sheath by replacing myelin producing cells or restoring their function.

Myelin related diseases or disorders which may be treated or ameliorated by the methods of the present invention include diseases, disorders or injuries which relate to dysmyelination or demyelination in a subject's brain cells, e.g., CNS neurons. Such diseases include, but are not limited to, diseases and disorders in which the myelin which surrounds the neuron is either absent, incomplete, not formed properly, or is deteriorating. Such disease include, but are not limited to, multiple sclerosis (MS), neuromyelitis optica (NMO), progressive multifocal leukoencephalopathy (PML), encephalomyelitis (EPL), central pontine myelolysis (CPM), adrenoleukodystrophy, Alexander's disease, Pelizaeus Merzbacher disease (PMD), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, Guillian-Barre syndrome, Marie-Charcot-Tooth disease and Bell's palsy.

Myelin related diseases or disorders which may be treated or ameliorated by the methods of the present invention include a disease or disorder characterized by a myelin deficiency. Insufficient myelination in the central nervous system has been implicated in a wide array of neurological disorders. Among these are forms of cerebral palsy in which a congenital deficit in forebrain myelination in children with periventricular leukomalacia, contributes to neurological morbidity (Goldman et al., 2008) Goldman, S. A., Schanz, S., and Windrem, M. S. (2008). Stem cell-based strategies for treating pediatric disorders of myelin. Hum Mol Genet. 17, R76-83. At the other end of the age spectrum, myelin loss and ineffective repair may contribute to the decline in cognitive function associated with senescence (Kohama et al., 2011) Kohama, S. G., Rosene, D. L., and Sherman, L. S. (2011) Age (Dordr). Age-related changes in human and non-human primate white matter: from myelination disturbances to cognitive decline. Therefore, it is contemplated that effective compositions and methods of enhancing myelination and/or remyelination may have substantial therapeutic benefits in halting disease progression and restoring function in a wide array of myelin-related disorders.

In some embodiments, the compositions of the present invention can be administered to a subject that does not have, and/or is not suspected of having, a myelin related disorder in order to enhance or promote a myelin dependent process. In some embodiments, compositions described herein can be administered to a subject to promote myelination of CNS neurons in order to enhance cognition, which is known to be a myelin dependent process, in cognitive healthy subjects. In certain embodiments, compositions described herein can be administered in combination with cognitive enhancing (nootropic) agents. Exemplary agents include any drugs, supplements, or other substances that improve cognitive function, particularly executive functions, memory, creativity, or motivation, in healthy individuals. Non limiting examples include racetams (e.g., piracetam, oxiracetam, and aniracetam), nutraceuticals (e.g., Bacopa monnieri, Panax ginseng, Ginko biloba, and GABA), stimulants (e.g., amphetamine pharmaceuticals, methylphenidate, eugeroics, xanthines, and nicotine), L-Theanine, Tolcapone, Levodopa, Atomoxetine, and Desipramine.

The overall dosage of a therapeutic agent (e.g., an RNA molecule, a polynucleotide encoding the RNA molecule, a vector genome, or a vector, such as an rAAV vector, or a cell) will be a therapeutically effective amount depending on several factors including the overall health of a subject, the subject's disease state, severity of the condition, the observation of improvements and the formulation and route of administration of the selected agent(s). Determination of a therapeutically effective amount is within the capability of those skilled in the art. The exact formulation, route of administration and dosage can be chosen by the individual physician in view of the subject's condition.

In certain embodiments, the cell or nucleotide compositions described herein may be administered in an amount effective to enhance myelin production in the CNS of a subject by an increase in the amount of myelin proteins (e.g., MBP) of at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level of myelin proteins of an untreated subject.

In other embodiments, the cell or nucleotide compositions may be administered in an amount effective to promote survival of CNS neurons in a subject by an increase in the number of surviving neurons of at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the number of surviving neurons in an untreated CNS neurons or subject.

Another strategy for treating a subject suffering from myelin-related disorder is to administer a therapeutically effective amount of a cell or nucleotide composition described herein along with a therapeutically effective amount of an oligodendrocyte differentiation and/or proliferation inducing agent(s) and/or anti-neurodegenerative disease agent. Examples of anti-neurodegenerative disease agents include L-dopa, cholinesterase inhibitors, anticholinergics, dopamine agonists, steroids, and immunomodulators including interferons, monoclonal antibodies, and glatiramer acetate. Therefore, in a further aspect of the disclosure, the compositions described herein can be administered as part of a combination therapy with adjunctive therapies for treating neurodegenerative and myelin related disorders.

The phrase “combination therapy” embraces the administration of oligodendrocyte precursor differentiation inducing compositions described herein and a therapeutic agent as part of a specific treatment regimen intended to provide a beneficial effect from the co-action of these therapeutic agents. When administered as a combination, the oligodendrocyte precursor differentiation inducing compound and a therapeutic agent can be formulated as separate compositions. Administration of these therapeutic agents in combination typically is carried out over a defined time period (usually minutes, hours, days or weeks depending upon the combination selected).

### 7. Pharmaceutical Compositions

The present disclosure provides a pharmaceutical composition, or medicament, for preventing or treating an inherited or acquired disorder of myelin. In some embodiments, a pharmaceutical composition comprises one or more of the above-described RNA molecule, polynucleotide, expression cassette, expression vector (e.g., viral vector genome, expression vector, rAAV vector), and host cell.

The pharmaceutical composition further comprises a pharmaceutically-acceptable carrier, adjuvant, diluent, excipient and/or other medicinal agents. A pharmaceutically acceptable carrier, adjuvant, diluent, excipient or other medicinal agent is one that is not biologically or otherwise undesirable, e.g., the material may be administered to a subject without causing undesirable biological effects which outweigh the advantageous biological effects of the material. Any suitable pharmaceutically acceptable carrier or excipient can be used in the preparation of a pharmaceutical composition according to the invention (See e.g., Remington The Science and Practice of Pharmacy, Adeboye Adejare (Editor) Academic Press, November 2020).

A pharmaceutical composition is typically sterile, pyrogen-free and stable under the conditions of manufacture and storage. A pharmaceutical composition may be formulated as a solution (e.g., water, saline, dextrose solution, buffered solution, or other pharmaceutically sterile fluid), microemulsion, liposome, or other ordered structure suitable to accommodate a high product (e.g., viral vector particles, microparticles or nanoparticles) concentration.

In some embodiments, a pharmaceutical composition comprising the above-described RNA molecule, polynucleotide, expression cassette, expression vector, vector genome, host cell or rAAV vector of the disclosure is formulated in water or a buffered saline solution. A carrier may be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by use of a coating such as lecithin, by maintenance of a required particle size, in the case of dispersion, and by the use of surfactants. In some embodiments, it may be preferable to include isotonic agents, for example, a sugar, a polyalcohol such as mannitol, sorbitol, or sodium chloride in the composition. Prolonged adsorption of an injectable composition can be brought about by including, in the composition, an agent which delays absorption, e.g., a monostearate salt and gelatin. In some embodiments, a nucleic acid, vector and/or host cell of the disclosure may be administered in a controlled release formulation, for example, in a composition which includes a slow-release polymer or other carrier that protects the product against rapid release, including an implant and microencapsulated delivery system.

In some embodiments, a pharmaceutical composition of the disclosure is a parenteral pharmaceutical composition, including a composition suitable for intravenous, intraarterial, subcutaneous, intradermal, intraperitoneal, intramuscular, intraarticular, intraparenchymal (IP), intrathecal (IT), intracerebroventricular (ICV) and/or intracisternal magna (ICM) administration. In some embodiments, a pharmaceutical composition of this disclosure is formulated for administration by ICV injection. In some embodiments, an rAAV vector (e.g., AAV/Olig001) is formulated in 350 mM NaCl and 5% D-sorbitol in PBS.

### 8. Methods of Administration

The above-described RNA molecule, or polynucleotide, or a vector (e.g., vector genome, rAAV vector) may be administered to a subject (e.g., a patient) in order to treat the subject. Administration of a vector to a human subject, or an animal in need thereof, can be by any means known in the art for administering a vector. A target cell of a vector of the present disclosure includes cells of the CNS, preferably oligodendrocytes.

A vector can be administered in addition to, and as an adjunct to, the standard of care treatment. That is, the vector can be co-administered with another agent, compound, drug, treatment or therapeutic regimen, either simultaneously, contemporaneously, or at a determined dosing interval as would be determined by one skilled in the art using routine methods. Uses disclosed herein include administration of an rAAV vector of the disclosure at the same time, in addition to and/or on a dosing schedule concurrent with, the standard of care for the disease as known in the art.

In some embodiments, a combination composition includes one or more immunosuppressive agents. In some embodiments, a combination composition includes an rAAV vector comprising a transgene (e.g., a polynucleotide encoding an RNA molecule disclosed herein) and one or more immunosuppressive agents. In some embodiments, a method includes administering or delivering an rAAV vector comprising the transgene to a subject and administering an immunosuppressive agent to the subject either prophylactically prior to administration of the vector, or after administration of the vector (i.e., either before or after symptoms of a response against the vector and/or the protein provided thereby are evident).

In one embodiment, a vector of the disclosure (e.g., an rAAV vector) is administered systemically. Exemplary methods of systemic administration include, but are not limited to, intravenous (e.g., portal vein), intraarterial (e.g., femoral artery, hepatic artery), intravascular, subcutaneous, intradermal, intraperitoneal, transmucosal, intrapulmonary, intralymphatic and intramuscular administration, and the like, as well as direct tissue or organ injection. One skilled in the art would appreciate that systemic administration can deliver a nucleic acid to all tissues. In some embodiments, direct tissue or organ administration includes administration to areas directly affected by oligodendrocyte deficiency (e.g., brain and/or central nervous system). In some embodiments, vectors of the disclosure, and pharmaceutical compositions thereof, are administered to the brain parenchyma (i.e., by intraparenchymal administration), to the spinal canal or the subarachnoid space so that it reaches the cerebrospinal fluid (CSF) (i.e., by intrathecal administration), to a ventricle of the brain (i.e., by intracerebroventricular administration) and/or to the cisterna magna of the brain (i.e., by intracisternal magna administration).

Accordingly, in some embodiments, a vector of the present disclosure is administered by direct injection into the brain (e.g., into the parenchyma, ventricle, cisterna magna, etc.) and/or into the CSF (e.g., into the spinal canal or subarachnoid space) to treat a disorder of myelin. A target cell of a vector of the present disclosure includes a cell located in the cortex, subcortical white matter of the corpus callosum, striatum and/or cerebellum. In some embodiments, a target cell of a vector of the present disclosure is an oligodendrocyte. Additional routes of administration may also comprise local application of a vector under direct visualization, e.g., superficial cortical application, or other stereotaxic application.

In some embodiments, a vector of the disclosure is administered by at least two routes. For example, a vector is administered systemically and also directly into the brain. If administered via at least two routes, the administration of a vector can be, but need not be, simultaneous or contemporaneous. Instead, administration via different routes can be performed separately with an interval of time between each administration.

The above-described RNA molecule, or polynucleotide encoding the RNA molecule, or a vector genome, or an rAAV vector comprising the polynucleotide may be used for transduction of a cell ex vivo or for administration directly to a subject (e.g., directly to the CNS of a patient with a disease). In some embodiments, a transduced cell (e.g., a host cell) is administered to a subject to treat or prevent a disease, disorder or condition (e.g., cell therapy for the disease). An rAAV vector comprising a therapeutic nucleic acid (e.g., encoding the RNA molecule) is preferably administered to a cell in a biologically-effective amount. In some embodiments, a biologically-effective amount of a vector is an amount that is sufficient to result in reducing the expression of a related gene in a target cell.

In some embodiments, the disclosure includes a method of decreasing the level and/or activity of a gene in a cell by administering to a cell (in vivo, in vitro or ex vivo) a polynucleotide encoding an RNA molecule described herein, either alone or in a vector (including a plasmid, a virus vector, a nanoparticle, a liposome, or any known method for providing a nucleic acid to a cell).

The dosage amount of an rAAV vector depends upon, e.g., the mode of administration, disease or condition to be treated, the stage and/or aggressiveness of the disease, individual subject's condition (age, sex, weight, etc.), particular viral vector, stability of protein to be expressed, host immune response to the vector, and/or gene to be delivered. Generally, doses range from at least 1×10⁸, or more, e.g., 1×10⁹, 1×10¹⁰, 1×10¹¹, 1×10¹², 1×10¹³, 1×10¹⁴, 1×10¹⁵ or more vector genomes (vg) per kilogram (kg) of body weight of the subject to achieve a therapeutic effect.

In some embodiments, a polynucleotide encoding an RNA molecule described herein may be administered as a component of a DNA molecule (e.g., a recombinant nucleic acid) having a regulatory element (e.g., a promoter) appropriate for expression in a target cell (e.g., oligodendrocytes). The polynucleotide may be administered as a component of a plasmid or a viral vector, such as an rAAV vector. An rAAV vector may be administered in vivo by direct delivery of the vector (e.g., directly to the CNS) to a patient in need of treatment. An rAAV vector may be administered to a patient ex vivo by administration of the vector in vitro to a cell from a donor patient in need of treatment, followed by introduction of the transduced cell back into the donor (e.g., cell therapy).

### 9. Kit

The present disclosure provides a kit with packaging material and one or more components therein. A kit typically includes a label or packaging insert including a description of the components or instructions for use in vitro, in vivo or ex vivo, of the components therein. A kit can contain a collection of such components, e.g., the above-described RNA molecule, polynucleotide, nucleic acid, expression cassette, expression vector (e.g., viral vector genome, expression vector, rAAV vector), and host cell, and optionally a second active agent such as a compound, therapeutic agent, drug or composition.

A kit refers to a physical structure that contains one or more components of the kit. Packaging material can maintain the components in a sterile manner and can be made of material commonly used for such purposes (e.g., paper, glass, plastic, foil, ampules, vials, tubes, etc).

A label or insert can include identifying information of one or more components therein, dose amounts, clinical pharmacology of the active ingredients(s) including mechanism of action, pharmacokinetics and pharmacodynamics. A label or insert can include information identifying manufacture, lot numbers, manufacture location and date, expiration dates. A label or insert can include information on a disease (e.g., an inherited or acquired disorder of myelin such as PMD, MSA, and H-ABC) for which a kit component may be used. A label or insert can include instructions for a clinician or subject for using one or more of the kit components in a method, use or treatment protocol or therapeutic regimen. Instructions can include dosage amounts, frequency of duration and instructions for practicing any of the methods, uses, treatment protocols or prophylactic or therapeutic regimens described herein.

A label or insert can include information on potential adverse side effects, complications or reaction, such as a warning to a subject or clinician regarding situations where it would not be appropriate to use a particular composition.

### 10. Definitions

As used herein, the terms “nucleic acid sequence,” “nucleotide sequence,” and “polynucleotide” refer interchangeably to any molecule composed of or comprising monomeric nucleotides connected by phosphodiester linkages. A nucleic acid may be an oligonucleotide or a polynucleotide. Nucleic acid sequences are presented herein in the direction from the 5′ to the 3′ direction. A nucleic acid sequence (i.e., a polynucleotide) of the present disclosure can be a deoxyribonucleic acid (DNA) molecule or ribonucleic acid (RNA) molecule and refers to all forms of a nucleic acid such as, double stranded molecules, single stranded molecules, small or short hairpin RNA (shRNA), micro interfering RNA or micro RNA (miRNA), small or short interfering RNA (siRNA), trans-splicing RNA, antisense RNA, messenger RNA, transfer RNA, ribosomal RNA. Where a polynucleotide is a DNA molecule, that molecule can be a gene, a cDNA, an antisense molecule or a fragment of any of the foregoing molecules. Nucleotides are indicated herein by a single letter code: adenine (A), guanine (G), thymine (T), cytosine (C), inosine (I) and uracil (U). A nucleotide sequence may be chemically modified or artificial. Nucleotide sequences include peptide nucleic acids (PNA), morpholinos and locked nucleic acids (LNA), as well as glycol nucleic acids (GNA) and threose nucleic acids (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′-P5′-phosphoramidates, and oligoribonucleotide phosphorothioates and their 2′-O-allyl analogs and 2′-O-methylribonucleotide methylphosphonates which may be used in a nucleotide sequence of the disclosure.

In some embodiments, a protein or a nucleic acid is isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.). In some embodiments, any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U, and U may be replaced with T.

“Heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene” refers to a heterologous polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

As used herein, the term “recombinant,” refers to a vector, polynucleotide (e.g., a recombinant nucleic acid), polypeptide or cell that is the product of various combinations of cloning, restriction or ligation steps (e.g. relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements). The terms respectively include replicates of the original polynucleotide construct and progeny of the original virus construct.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences. Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

As used herein, the term “vector” refers to a plasmid, virus (e.g., an rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes an RNA, or a polypeptide or a protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a recombinant nucleic acid sequence that is packaged or encapsidated to form an rAAV vector. Typically, a vector genome includes a heterologous polynucleotide sequence, e.g., a transgene, regulatory elements, ITRs not originally present in the capsid. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is typically referred to as the “plasmid backbone,” which is important for cloning. selection and amplification of the plasmid, a process that is needed for propagation of recombinant viral vector production, but which is not itself packaged or encapsidated into an rAAV vector.

As used herein, the term “viral vector” generally refers to a viral particle that functions as a nucleic acid delivery vehicle and which comprises a vector genome (e.g., comprising a transgene instead of a nucleic acid encoding an AAV rep and cap) packaged within the viral particle (i.e., capsid) and includes, for example, lenti- and parvo-viruses, including AAV serotypes and variants (e.g., rAAV vectors). A recombinant viral vector does not comprise a vector genome comprising a rep and/or a cap gene.

As used herein “miRNA scaffold” may refer to a polynucleotide containing (i) a double-stranded sequence targeting a gene of interest for knockdown by RNAi and (ii) additional sequences that form a stem-loop structure resembling that of endogenous miRNAs. A sequence targeting a gene of interest for RNAi (e.g., a short, about 20-nt sequence) may be ligated to sequences that create a miRNA-like stem-loop and a sequence that base pairs with the sequence of interest to form a duplex when the polynucleotide is assembled into the miRNA-like secondary structure. As described herein, this duplex may hybridize imperfectly, e.g., it may contain one or more unpaired or mispaired bases. Upon cleavage of this polynucleotide by Dicer, this duplex containing the sequence targeting a gene of interest may be unwound and incorporated into the RISC complex. A miRNA scaffold may refer to the miRNA itself or to a DNA polynucleotide encoding the miRNA. An example of a miRNA scaffold is the miR-155 sequence (Lagos-Quintana, M. et al. (2002) Curr. Biol. 12:735-9). Commercially available kits for cloning a sequence into a miRNA scaffold are known in the art (e.g., the INVITROGEN BLOCK-IT Pol II miR RNAi expression vector kit from Life Technologies, Thermo Fisher Scientific; Waltham, Mass.).

A functional variant or equivalent of a reference peptide, polypeptide, or protein refers to a polypeptide derivative of the reference peptide, polypeptide, or protein, e.g., a protein having one or more point mutations, insertions, deletions, truncations, a fusion protein, or a combination thereof. It retains substantially the activity to of the reference peptide, polypeptide, or protein. In general, the functional equivalent is at least 60% (e.g., any number between 60% and 100%, inclusive, e.g., 60%, 70%, 80%, 85%, 90%, 95%, and 99%) identical to the reference peptide, polypeptide, or protein. In certain embodiments, a point mutation can be a conservative modification.

As used herein, the term “conservative modification” refers to amino acid modifications that do not significantly affect or alter the biological characteristics of a polypeptide or protein. Such conservative modifications include amino acid substitutions, additions and deletions. Modifications can be introduced into a polypeptide or protein by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine) and aromatic side chains (e.g., tyrosine, phenylalanine, tryptophan, histidine). Non-conservative substitutions will entail exchanging a member of one of these classes for another class.

As used herein, the terms “treat,” “treating” or “treatment” refer to administration of a therapy that partially or completely alleviates, ameliorates, relieves, inhibits, delays onset of, reduces severity of, and/or reduces incidence of one or more symptoms, features, and/or causes of a particular disease, disorder, and/or condition.

As used herein, the term “ameliorate” means a detectable or measurable improvement in a subject's disease, disorder or condition, or symptom thereof, or an underlying cellular response. A detectable or measurable improvement includes a subjective or objective decrease, reduction, inhibition, suppression, limit or control in the occurrence, frequency, severity, progression or duration of, complication cause by or associated with, improvement in a symptom of, or a reversal of a disease, disorder or condition.

As used herein, the term “associated with” refers to with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population).

As used herein, the term “prevent” or “prevention” refers to delay of onset, and/or reduction in frequency and/or severity of one or more sign or symptom of a particular disease, disorder or condition (e.g., a myelin disease). In some embodiments, prevention is assessed on a population basis such that an agent is considered to “prevent” a particular disease, disorder or condition if a statistically significant decrease in the development, frequency and/or intensity of one or more sign or symptom of the disease, disorder or condition is observed in a population susceptible to the disease, disorder or condition. Prevention may be considered complete when onset of disease, disorder or condition has been delayed for a predefined period of time.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a subject is a non-human disease model. In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional myelin. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered.

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

### EXAMPLES

### Example 1. Construction of AAV Expression Cassette

The 774 base pair coding sequence of human proteolipid protein 1 (PLP1), transcript variant 1, mRNA (NCBI Reference Sequence: NM_000533.5; SEQ ID NO: 1) was obtained from the National Center for Biotechnology Information (NCBI) ncbi.nlm.nih.gov/refseq/.

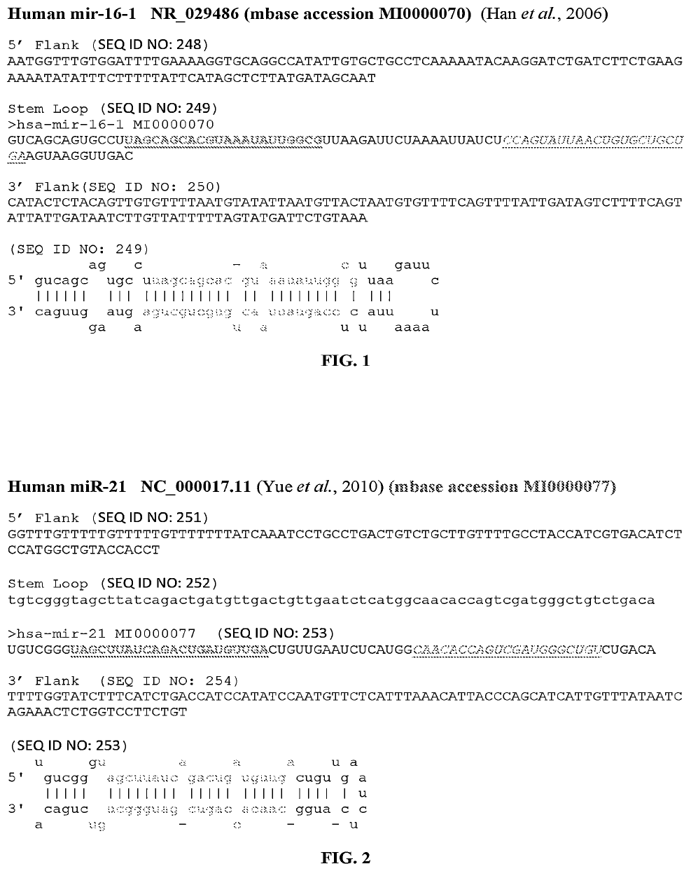

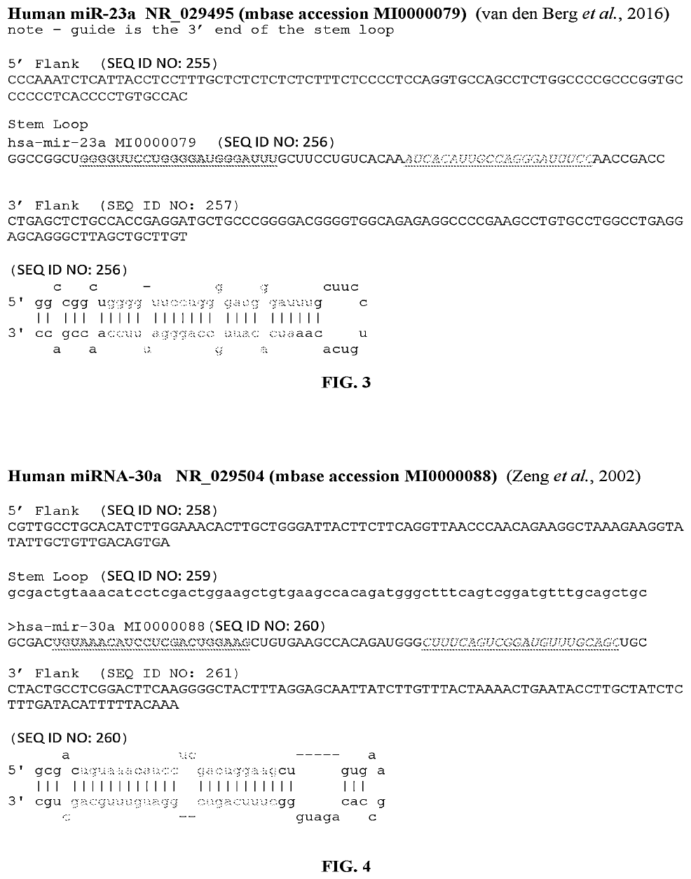

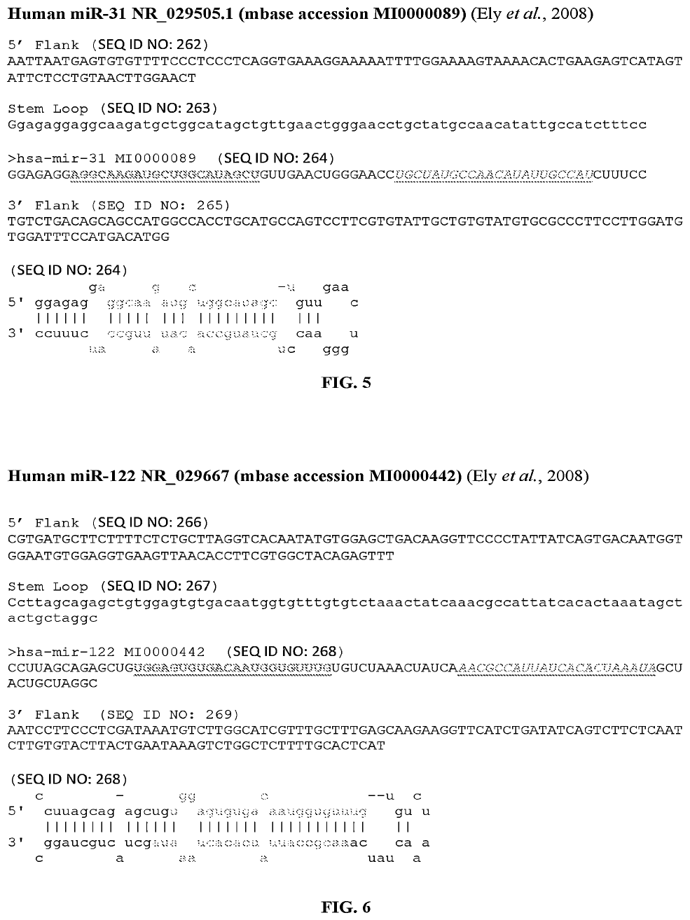

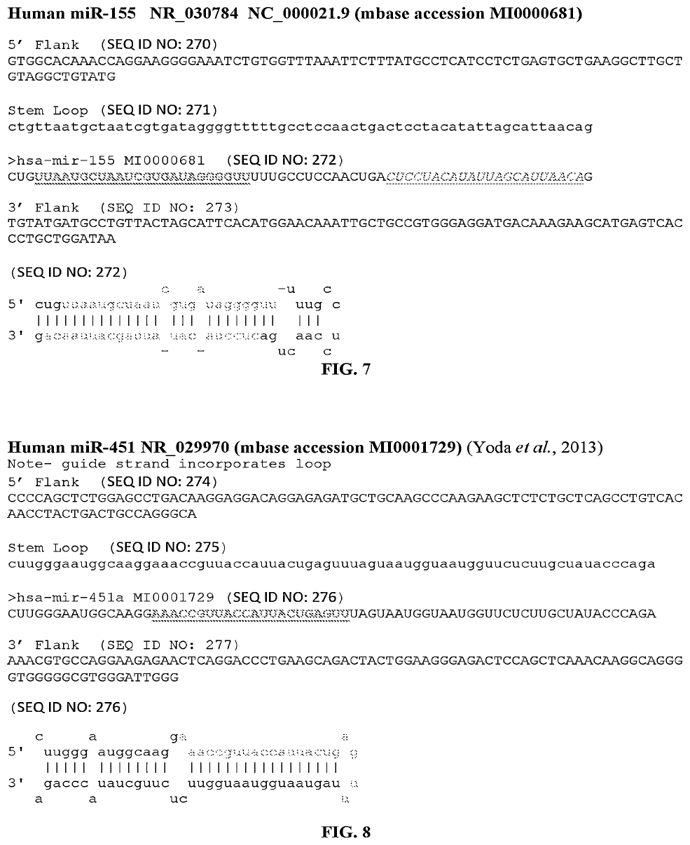

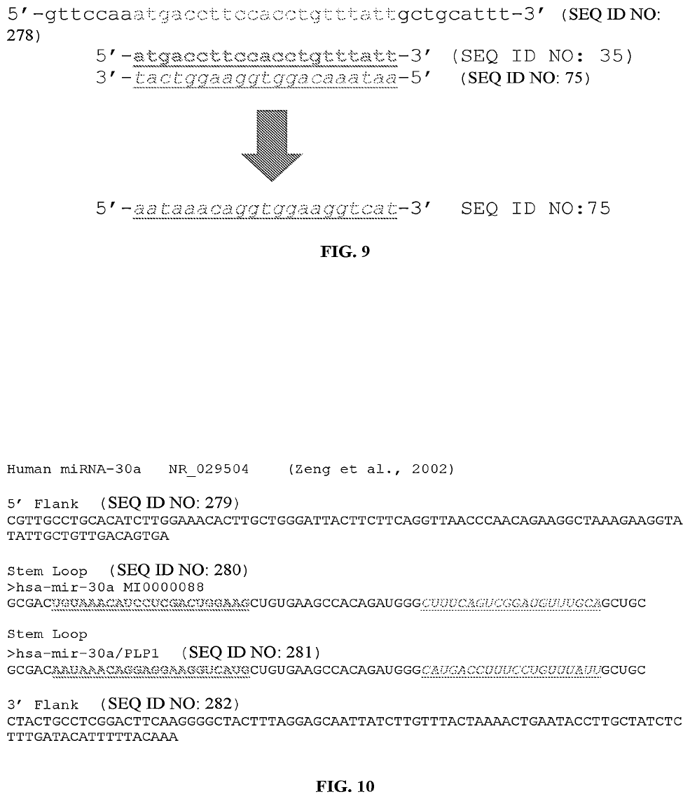

Non-commercial web-based algorithm-based design tools were used to identify target regions in the coding sequence of the gene of interest (See FIG. 1, SEQ ID NO: 35 sequence in red). Such design tools include Designer of Small Interfering RNA (DSIR) (Vert et al., 2006) and the Genetic Perturbation Platform made available by the Broad Institute. Design tools are used to generate 19-21 nucleotide target sequences in the human PLP1 coding sequence. The nucleotide sequence that is perfectly complementary to the target sequence was determined (See FIG. 9, SEQ ID NO: 75 sequence in blue, italicized and underlined, aligned 3′ to 5′ to demonstrate complementarity). In this example, human miR-30a was used as a backbone (see FIG. 4) to design the artificial microRNA, which includes the 3′ and 5′ flanking regions (of 50-100 nucleotides) as well as the loop region. The 20-22-nucleotide sequence that is perfectly complementary to the target sequence is placed immediately after 5′ flank sequence, followed by the loop sequence from the naturally occurring microRNA, the reverse complement of the 20-22 nucleotide guide strand (modified by deletion in positions 10-11 to create a bulge which permits preferential loading of the guide strand into the RISC complex), then followed by the 3′ flank sequence (FIG. 10).

Folding of the sequences of both the endogenous miRNA (miR-30a in this experiment) and the designed sequence (miR-30a/PLP1 incorporating SEQ ID NO: 75) are predicted using web-based software (mfold) for the prediction of the secondary structure of single stranded nucleic acids (Zuker, 2003). In this case, the designed pre-miRNA shares the same secondary structure as the native sequence (having the same framework regions, but different guide and strand sequences) and has a similar free energy (dG) as the native sequence (FIG. 11). The resulting hairpin has a conserved loop region and contains the cleavage sites needed for Dicer to remove the loop and leave the dsRNA duplex.

The designed oligonucleotide is synthesized and cloned into a plasmid containing the promoter of choice. A cell line able to expresses the PLP1 gene is selected. A common choice is human embryonic kidney cells HEK293. Transfection is performed and knockdown is assessed.

### Example 2. Additional AAV Expression Cassettes

Additional AAV expression cassettes are made in the same manner described in Example 1 using other suitable endogenous human miRNA structures as backbones or scaffolds. These human miRNA structures include Human mir-16-1 NR_029486 (mbase accession MI0000070) (Han et al., 2006), Human miR-21 NC_000017.11 (Yue et al., 2010) (mbase accession MI0000077), Human miR-23a NR_029495 (mbase accession MI0000079) (van den Berg et al., 2016), Human miRNA-30a NR_029504 (mbase accession MI0000088) (Zeng et al., 2002), Human miR-31 NR_029505.1 (mbase accession MI0000089) (Ely et al., 2008), Human miR-122 NR_029667 (mbase accession MI0000442) (Ely et al., 2008), Human miR-155 NR_030784 NC_000021.9 (mbase accession MI0000681), and Human miR-451 NR_029970 (mbase accession M10001729) (Yoda et al., 2013).

### Example 3 miRNA Down Regulation of PLP1 Protein Expression

This example demonstrates that the miRNA constructs can reduce the expression of a target protein. In the experiment, the abilities of three artificial Plp1-miRNAs to inhibit Plp1 protein expression were evaluated in vitro. As described above in Example 1, target regions of the PLP1 coding sequence (SEQ ID NO: 1) were identified using non-commercial web-based algorithm-based design tools for miRNA formation. Three target sequences and guide strands for targeting PLP1 were selected for targeting with miRNAs (SEQ ID NO: 14 and 54; SEQ ID NO: 34 and 74; and SEQ ID NO: 35 and 75). These three sets of target sequences and guide strands were incorporated into miR30-based shRNA knockdown vectors (constructed as plasmids in E. coli). The three plasmids each contained a polycistronic expression cassette consisting of a CBh promoter, one of the three miR30-based shRNA sequences targeting human Plp1 mRNA, and a codon optimized enhanced green fluorescent protein (eGFP) cloned between the two inverted terminal repeats (ITRs). A fourth plasmid contained a scrambled shRNA (none in human or mouse) and served as a control. Plasmid 1332 contained a miR30-based shRNA knockdown vector with SEQ ID NO: 35 and 75, plasmid 1333 contained a miR30-based shRNA knockdown vector with SEQ ID NO: 14 and 54, and plasmid 1336 contained a miR30-based shRNA knockdown vector with SEQ ID NO: 34 and 74. Plasmid 1307 contained a scrambled shRNA (not targeting any mRNA in human or mouse) and served as a control.

Knockdown efficiency was evaluated in vitro by co-transfection with a PLP1 cDNA expression plasmid vector in L cells using Lipofectamine300. 72 hours later, Western blotting was performed on cell lysates to evaluate the expression of PLP1 protein (using alpha-tubulin as a control). Ten ug of protein per lane was electrophoresed on SDS-polyacrylamide gels and transferred to nitrocellulose membranes. Western blotting was performed using standard techniques known to one of skill in the art. Briefly, blocked membranes were incubated overnight with Rat anti-PLP AA3 (1:250 dilution), then with an anti-Rat horseradish peroxidase-coupled secondary antibody (1:1000 dilution). Blots were washed and immunodetection was performed.

The results of Western blotting, where PLP protein runs as two bands, a monomer at approximately 22 kDa and a dimer at 44 kDa, show that at 72 hours, plasmids 1332 (containing a miR30-based shRNA knockdown vector with SEQ ID NO: 35 and 75) and plasmid 1333 (containing a miR30-based shRNA knockdown vector with SEQ ID NO: 14 and 54) achieved a significant reduction of PLP1 expression, whereas plasmid 1336 (containing a miR30-based shRNA knockdown vectors with SEQ ID NO: 34 and 74) did not.

A morphometric analysis to show fold changes was carried out using data obtained from 3 repeated experiments in which the two PLP1 bands at 22 kD and 44 kD were combined and normalized by eGFP from the blots. The results demonstrated quantitatively that artificial PLP1 miRNAs suppressed PLP1 protein expression in vitro. Specifically, plasmids 1332 and 1333, containing miR30-based shRNA knockdown vectors (with SEQ ID NO: 35 and 75 and SEQ ID NO: 14 and 54, respectively) achieved a significant reduction of PLP1 expression, whereas plasmids 1336, containing miR30-based shRNA knockdown vectors (with SEQ ID NO: 34 and 74) did not.

Statistical significance was determined using one-way ANOVA followed by Tukey's multiple comparison test. Both plasmid 1332 and plasmid 1333 resulted in more than 50% reduction as compared to plasmid 1307 (P<0.01) and to plasmid 1336 (P<0.001). The greatest reduction was achieved by plasmid 1333 containing the miR30-based shRNA knockdown vector (with SEQ ID NO: 14 and 54, shown in FIG. 12).

### REFERENCES

Chen H, McCarty D M, Bruce A T, Suzuki K, and Suzuki K (1998) Gene transfer and expression in oligodendrocytes under the control of myelin basic protein transcriptional control region mediated by adeno-associated virus. Gene Ther 5:50-58.
Curiel J, Rodriguez Bey G, Takanohashi A, Bugiani M, Fu X, Wolf N I, Nmezi B, Schiffmann R, Bugaighis M, Pierson T, Helman G, Simons C, van der Knaap M S, Liu J, Padiath Q, and Vanderver A (2017) TUBB4A mutations result in specific neuronal and oligodendrocytic defects that closely match clinically distinct phenotypes. Hum Mol Genet 26:4506-4518.
Elitt M S, Barbar L, Shick H E, Powers B E, Maeno-Hikichi Y, Madhavan M, Allan K C, Nawash B S, Gevorgyan A S, Hung S, Nevin Z S, Olsen H E, Hitomi M, Schlatzer D M, Zhao H T, Swayze A, LePage D F, Jiang W, Conlon R A, Rigo F, and Tesar P J (2020) Suppression of proteolipid protein rescues Pelizaeus-Merzbacher disease. Nature 585:397-403.
Ely A, Naidoo T, Mufamadi S, Crowther C, and Arbuthnot P (2008) Expressed Anti-HBV Primary MicroRNA Shuttles Inhibit Viral Replication Efficiently In vitro and In Vivo. Molecular Therapy 16:1105-1112.
Fire A, Xu S, Montgomery M K, Kostas S A, Driver S E, and Mello C C (1998) Potent and specific genetic interference by double-stranded RNA in Caenorhabditis elegans. Nature 391:806-811.
Francis J S, Markov V, Wojtas I D, Gray S, McCown T, Samulski R J, Figueroa M, and Leone P (2021) Preclinical biodistribution, tropism, and efficacy of oligotropic AAV/Olig001 in a mouse model of congenital white matter disease. Mol Ther Methods Clin Dev 20:520-534.
Han J, Lee Y, Yeom K-H, Nam J-W, Heo I, Rhee J-K, Sohn S Y, Cho Y, Zhang B-T, and Kim V N (2006) Molecular basis for the recognition of primary microRNAs by the Drosha-DGCR8 complex. Cell 125:887-901.
Keeler A, ElMallah M, and Flotte T (2017) Gene Therapy 2017: Progress and Future Directions: Gene Therapy 2017: Progress and Future Directions. Clinical and Translational Science 10:242-248.
Kotowska-Zimmer A, Pewinska M, and Olejniczak M (2021) Artificial miRNAs as therapeutic tools: Challenges and opportunities. Wiley Interdisciplinary Reviews: RNA 12:e1640, John Wiley & Sons, Ltd.
Lawlor P A, Bland R J, Mouravlev A, Young D, and During M J (2009) Efficient gene delivery and selective transduction of glial cells in the mammalian brain by AAV serotypes isolated from nonhuman primates. Mol Ther 17:1692-1702.
Li H, Okada H, Suzuki S, Sakai K, Izumi H, Matsushima Y, Ichinohe N, Goto Y-I, Okada T, and Inoue K (2019) Gene suppressing therapy for Pelizaeus-Merzbacher disease using artificial microRNA. JCI Insight 4:125052.
Mandel R J, Marmion D J, Kirik D, Chu Y, Heindel C, McCown T, Gray S J, and Kordower J H (2017) Novel oligodendroglial alpha synuclein viral vector models of multiple system atrophy: studies in rodents and nonhuman primates. Acta Neuropathol Commun 5:47.
Marmion D J, Peelaerts W, and Kordower J H (2021) A historical review of multiple system atrophy with a critical appraisal of cellular and animal models. J Neural Transm, doi: 10.1007/s00702-021-02419-8.
Mavroeidi P, Arvanitaki F, Karakitsou A-K, Vetsi M, Kloukina I, Zweckstetter M, Giller K, Becker S, Sorrentino Z A, Giasson B I, Jensen P H, Stefanis L, and Xilouri M (2019) Endogenous oligodendroglial alpha-synuclein and TPPP/p25α orchestrate alpha-synuclein pathology in experimental multiple system atrophy models. Acta Neuropathol 138:415-441.
Multiple System Atrophy Fact Sheet|National Institute of Neurological Disorders and Stroke (n.d.).
Osório MJ, and Goldman S A (2018) Neurogenetics of Pelizaeus-Merzbacher disease. Handb Clin Neurol 148:701-722.
Powell S K, Khan N, Parker C L, Samulski R J, Matsushima G, Gray S J, and McCown T J (2016) Characterization of a novel adeno-associated viral vector with preferential oligodendrocyte tropism. Gene Ther 23:807-814.
Sase S, Almad A A, Boecker C A, Guedes-Dias P, Li J J, Takanohashi A, Patel A, McCaffrey T, Patel H, Sirdeshpande D, Curiel J, Shih-Hwa Liu J, Padiath Q, Holzbaur E L, Scherer S S, and Vanderver A (2020) TUBB4A mutations result in both glial and neuronal degeneration in an H-ABC leukodystrophy mouse model. Elife 9:e52986.
Schmidt J L, Pizzino A, Nicholl J, Foley A, Wang Y, Rosenfeld J A, Mighion L, Bean L, da Silva C, Cho M T, Truty R, Garcia J, Speare V, Blanco K, Powis Z, Hobson G M, Kirwin S, Krock B, Lee H, Deignan J L, Westemeyer M A, Subaran R L, Thiffault I, Tsai E A, Fang T, Helman G, and Vanderver A (2020) Estimating the relative frequency of leukodystrophies and recommendations for carrier screening in the era of next-generation sequencing. Am J Med Genet A 182:1906-1912.
Simons C, Wolf N I, McNeil N, Caldovic L, Devaney J M, Takanohashi A, Crawford J, Ru K, Grimmond S M, Miller D, Tonduti D, Schmidt J L, Chudnow R S, van Coster R, Lagae L, Kisler J, Sperner J, van der Knaap M S, Schiffmann R, Taft R J, and Vanderver A (2013) A de novo mutation in the β-tubulin gene TUBB4A results in the leukoencephalopathy hypomyelination with atrophy of the basal ganglia and cerebellum. Am J Hum Genet 92:767-773.
Stadelmann C, Timmler S, Barrantes-Freer A, and Simons M (2019) Myelin in the Central Nervous System: Structure, Function, and Pathology. Physiological Reviews 99:1381-1431, American Physiological Society.
Toro Cabrera G, and Mueller C (2016) Design of shRNA and miRNA for Delivery to the CNS. Methods Mol Biol 1382:67-80.
van den Berg F T, Rossi J J, Arbuthnot P, and Weinberg M S (2016) Design of Effective Primary MicroRNA Mimics With Different Basal Stem Conformations. Mol Ther Nucleic Acids 5:e278.
Vert J-P, Foveau N, Lajaunie C, and Vandenbrouck Y (2006) An accurate and interpretable model for siRNA efficacy prediction. BMC Bioinformatics 7:520.
Wenning G K, Stefanova N, Jellinger K A, Poewe W, and Schlossmacher M G (2008) Multiple system atrophy: A primary oligodendrogliopathy. Annals of Neurology 64:239-246.
Williams G P, Marmion D J, Schonhoff A M, Jurkuvenaite A, Won W-J, Standaert D G, Kordower J H, and Harms A S (2020) T cell infiltration in both human multiple system atrophy and a novel mouse model of the disease. Acta Neuropathol 139:855-874.
Yoda M, Cifuentes D, Izumi N, Sakaguchi Y, Suzuki T, Giraldez A J, and Tomari Y (2013) PARN mediates 3′-end trimming of Argonaute2-cleaved precursor microRNAs. Cell Rep 5:10.1016/j.celrep.2013.09.029.
Yue J, Sheng Y, Ren A, and Penmatsa S (2010) A miR-21 hairpin structure-based gene knockdown vector. Biochem Biophys Res Commun 394:667-672.
Zeng Y, Wagner E J, and Cullen B R (2002) Both Natural and Designed Micro RNAs Can Inhibit the Expression of Cognate mRNAs When Expressed in Human Cells. Molecular Cell 9:1327-1333, Elsevier.
Zuker M (2003) Mfold web server for nucleic acid folding and hybridization prediction. Nucleic Acids Res 31:3406-3415.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method of reducing expression of a target gene in an oligodendrocyte or of treating an inherited or acquired disorder of myelin, the method comprising:
providing an adeno-associated virus (AAV) particle with preferential tropism for the oligodendrocyte cell surface; wherein the AAV particle encapsidates a nucleic acid that comprises from 5′ to 3′:
a 5′ inverted terminal repeat (ITR),
a promoter sequence region,
a polynucleotide encoding a pri- or pre-miRNA targeting said target gene, and
a 3′ ITR, and
contacting the AAV particle with the oligodendrocyte.

2. The method of claim 1, wherein the nucleic acid further comprises one or more of a post-transcriptional regulatory element and a polyA signal sequence region between the polynucleotide and the 3′ ITR.

3. The method of claim 1, wherein the pri- or pre-miRNA comprises
(a) a pri- or pre-miRNA scaffold,
(b) a heterologous guide strand, and
(c) a heterologous passenger strand.

4. The method of claim 3, wherein the pri- or pre-miRNA scaffold is a human pri- or pre-miRNA scaffold derived from a human miRNA.

5. (canceled)

6. (canceled)

7. (canceled)

8. The method of claim 3, wherein the heterologous guide strand is complementary to the mRNA of the target gene.

9. (canceled)

10. The method of claim 3, wherein the heterologous guide strand is complementary to the mRNA of a protein the elimination of which improves a treatment outcome of the inherited or acquired disorder of myelin.

11. The method of claim 10, wherein the inherited or acquired disorder of myelin is Pelizaeus-Merzbacher disease, multiple system atrophy, or Hypomyelination with Atrophy of the Basal Ganglia and Cerebellum (H-ABC).

12. The method of claim 11, wherein the protein is PLP1, alpha-synuclein, or microtubule associated protein tubulin beta-4a.

13. The method of claim 12, wherein the heterologous guide strand comprises a nucleotide sequence having at least 90% identity to one of SEQ ID NOs: 42 to 80, SEQ ID NOs: 122 to 161, or SEQ ID NOs: 204 to 244.

14. (canceled)

15. (canceled)

16. (canceled)

17. (canceled)

18. (canceled)

19. (canceled)

20. (canceled)

21. An RNA molecule comprising a first RNA sequence and a second RNA sequence, wherein the first and second RNA sequence are substantially complementary, wherein the first RNA sequence has a sequence length of at least 19 nucleotides and is at least 90% complementarity to one selected from the group consisting of SEQ ID NOs: 2-40, 82-121, and 163-203.

22. The RNA molecule of claim 21, wherein the RNA molecule is comprised in a pre-miRNA scaffold or a pri-miRNA scaffold derived from a human microRNA.

23. (canceled)

24. (canceled)

25. (canceled)

26. (canceled)

27. A polynucleotide encoding the RNA molecule of claim 21.

28. (canceled)

29. An expression cassette or expression vector comprising the polynucleotide of claim 27, comprising from 5′ to 3′ one or more of:
a 5′ inverted terminal repeat (ITR),
a promoter sequence region,
a post-transcriptional regulatory element,
a polyA signal sequence region, and
a 3′ ITR.

30. The expression vector of claim 29, wherein the expression vector is a viral vector.

31. The expression vector of claim 30, wherein the viral vector is an AAV vector.

32. The expression vector of claim 31, wherein the AAV vector has a preferential tropism for an oligodendrocyte cell or wherein said AAV vector is AAV-Olig001, AAV-Olig002, AAV-Olig003, or AAV9.

33. (canceled)

34. A host cell comprising the polynucleotide of claim 27 or an expression cassette or expression vector comprising said polynucleotide.

35. A pharmaceutical composition comprising
the RNA molecule of claim 21, or a polynucleotide encoding said RNA molecule, or an expression cassette or expression vector comprising said polynucleotide, or a host cell comprising said polynucleotide or said expression cassette or expression vector, and
a pharmaceutically acceptable carrier.

36. A method of treating an inherited or acquired disorder of myelin, comprising administering to a subject in need thereof the RNA molecule of claim 21, or a polynucleotide encoding said RNA molecule, or an expression cassette or expression vector comprising said polynucleotide, or a host cell comprising said polynucleotide or said expression cassette or expression vector, or a pharmaceutical composition comprising said RNA molecule, said polynucleotide, said expression cassette or expression vector, or said host cell.

37. The method of claim 36, wherein the subject is a human.

38. The method of claim 36, wherein the administration is via injection.

39. The method of claim 36, wherein the RNA molecule, or the polynucleotide, or the expression cassette, or the expression vector, or the host cell, or the pharmaceutical composition is (i) administered to a region of the central nervous system selected from the group consisting of brain parenchyma, spinal canal, subarachnoid space, a ventricle of the brain, cisterna magna and a combination thereof or (ii) administered by a method selected from the group consisting of intraparenchymal administration, intrathecal administration, intracerebroventricular administration, intracisternal magna administration and a combination thereof.

40. (canceled)

41. The method of claim 10, wherein the disorder is Alexander disease, Mitchell disease, autosomal dominant leukodystrophy with autonomic diseases (ADLD), central dysmyelinating leukodystrophy, Waardenburg syndrome, Hirschsprung disease, adult Polyglucosan Body Disease (APBD), hereditary diffuse leukoencephalopathy with spheroids, Aicardi-Goutieres syndrome, Canavan disease, Krabbe disease, Globoid cell leukodystrophy, X-linked adrenoleukodystrophy, Metachromatic leukodystrophy, hypomyelinating leukodystrophy-2, Niemann-Pick disease type C, 4H Leukodystrophy/Pol III-related leukodystrophy, Zellweger Spectrum Disorders, Childhood ataxia with central nervous system hypomyelination, Cerebrotendinous xanthomatosis, SOX10-associated peripheral demyelinating neuropathy, Adult Refsum disease, Autism Spectrum Disorder, Alzheimer's disease, Parkinson's disease, Fragile X syndrome, schizophrenia, multiple sclerosis, neuromyelitis optica, progressive multifocal leukoencephalopathy, encephalomyelitis, central pontine myelolysis, adrenoleukodystrophy, Wallerian Degeneration, optic neuritis, transverse myelitis, amyotrophic lateral sclerosis, Huntington's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Marie-Charcot-Tooth disease, or Bell's palsy.

42. (canceled)


SEQUENCE LISTING (285 sequences)

SEQ ID NO: 1 - DNA, 774 bp, Homo sapiens
  source 1..774: mol_type = unassigned DNA
atgggcttgttagagtgctgtgcagtggcactgttctgtggctgtggacatgaagccctcactggcacagaaaagctaattgagacctatttctccaaaaactaccaagactatgagtatctcatcaatgtgatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaagtttgtgggcatcacctatgccctgaccgttgtgtggctcctggtgtttgcctgctctgctgtgcctgtgtacatttacttcaacacctggaccacctgccagtctattgccttccccagcaagacctctgccagtataggcagtctctgtgctgatgccagaatgtatggtgttctcccatggaatgctttccctggcaaggtttgtggctccaaccttctgtccatctgcaaaacagctgagttccaaatgaccttccacctgtttattgctgcatttgtgggggctgcagctacactggtttccctgctcaccttcatgattgctgccacttacaactttgccgtccttaaactcatgggccgaggcaccaagttctga

SEQ ID NO: 2 - DNA, 21 bp, Homo sapiens
  source 1..21: mol_type = unassigned DNA
ctaccaagactatgagtatct

SEQ ID NO: 3 - DNA, 21 bp, Homo sapiens
  source 1..21: mol_type = unassigned DNA
actaccaagactatgagtatc

SEQ ID NO: 4 - DNA, 21 bp, Homo sapiens
  source 1..21: mol_type = unassigned DNA
caagactatgagtatctcatc

SEQ ID NO: 5 - DNA, 21 bp, Homo sapiens
  source 1..21: mol_type = unassigned DNA
gactatgagtatctcatcaat

SEQ ID NO: 6 - DNA, 21 bp, Homo sapiens
  source 1..21: mol_type = unassigned DNA
actatgagtatctcatcaatg

SEQ ID NO: 7 - DNA, 21 bp, Homo sapiens
  source 1..21: mol_type = unassigned DNA
gagtatctcatcaatgtgatc

SEQ ID NO: 8 - DNA, 21 bp, Homo sapiens
  source 1..21: mol_type = unassigned DNA
tgatccatgccttccagtatg

SEQ ID NO: 9 - DNA, 21 bp, Homo sapiens
  source 1..21: mol_type = unassigned DNA
ccttccagtatgtcatctatg

SEQ ID NO: 10 - DNA, 21 bp, Homo sapiens
  source 1..21: mol_type = unassigned DNA
cagtatgtcatctatggaact

SEQ ID NO: 11 - DNA, 21 bp, Homo sapiens
  source 1..21: mol_type = unassigned DNA
atctatggaactgcctctttc

SEQ ID NO: 12 - DNA, 21 bp, Homo sapiens
  source 1..21: mol_type = unassigned DNA
cctctttcttcttcctttatg

SEQ ID NO: 13 - DNA, 21 bp, Homo sapiens
  source 1..21: mol_type = unassigned DNA
tgcctctttcttcttccttta

SEQ ID NO: 14 - DNA, 21 bp, Homo sapiens
  source 1..21: mol_type = unassigned DNA
gcctctttcttcttcctttat

SEQ ID NO: 15 - DNA, 21 bp, Homo sapiens
  source 1..21: mol_type = unassigned DNA
cctctttcttcttcctttatg

SEQ ID NO: 16 - DNA, 21 bp, Homo sapiens
  source 1..21: mol_type = unassigned DNA
ggcgactacaagaccaccatc

SEQ ID NO: 17 - DNA, 21 bp, Homo sapiens
  source 1..21: mol_type = unassigned DNA
gcgactacaagaccaccatct

SEQ ID NO: 18 - DNA, 21 bp, Homo sapiens
  source 1..21: mol_type = unassigned DNA
aggccaacatcaagctcattc

SEQ ID NO: 19 - DNA, 21 bp, Homo sapiens
  source 1..21: mol_type = unassigned DNA
caacatcaagctcattctttg

SEQ ID NO: 20 - DNA, 21 bp, Homo sapiens
  source 1..21: mol_type = unassigned DNA
tttggagcgggtgtgtcattg

SEQ ID NO: 21 - DNA, 21 bp, Homo sapiens
  source 1..21: mol_type = unassigned DNA
gagcgggtgtgtcattgtttg

SEQ ID NO: 22 - DNA, 21 bp, Homo sapiens
  source 1..21: mol_type = unassigned DNA
ggtgtgtcattgtttgggaaa

SEQ ID NO: 23 - DNA, 21 bp, Homo sapiens
  source 1..21: mol_type = unassigned DNA
ggctaggacatcccgacaagt

SEQ ID NO: 24 - DNA, 21 bp, Homo sapiens
  source 1..21: mol_type = unassigned DNA
aggacatcccgacaagtttgt

SEQ ID NO: 25 - DNA, 21 bp, Homo sapiens
  source 1..21: mol_type = unassigned DNA
agtttgtgggcatcacctatg

SEQ ID NO: 26 - DNA, 21 bp, Homo sapiens
  source 1..21: mol_type = unassigned DNA
gctctgctgtgcctgtgtaca

SEQ ID NO: 27 - DNA, 21 bp, Homo sapiens
  source 1..21: mol_type = unassigned DNA
gctgtgcctgtgtacatttac

SEQ ID NO: 28 - DNA, 21 bp, Homo sapiens
  source 1..21: mol_type = unassigned DNA
ctgctgtgcctgtgtacattt

SEQ ID NO: 29 - DNA, 21 bp, Homo sapiens
  source 1..21: mol_type = unassigned DNA
ctgtgcctgtgtacatttact

SEQ ID NO: 30 - DNA, 21 bp, Homo sapiens
  source 1..21: mol_type = unassigned DNA
tgctgtgcctgtgtacattta

SEQ ID NO: 31 - DNA, 21 bp, Homo sapiens
  source 1..21: mol_type = unassigned DNA
gctgtgcctgtgtacatttac

SEQ ID NO: 32 - DNA, 21 bp, Homo sapiens
  source 1..21: mol_type = unassigned DNA
ctgtgtacatttacttcaaca

SEQ ID NO: 33 - DNA, 21 bp, Homo sapiens
  source 1..21: mol_type = unassigned DNA
tggaccacctgccagtctatt

SEQ ID NO: 34 - DNA, 21 bp, Homo sapiens
  source 1..21: mol_type = unassigned DNA
agcaagacctctgccagtata

SEQ ID NO: 35 - DNA, 21 bp, Homo sapiens
  source 1..21: mol_type = unassigned DNA
atgaccttccacctgtttatt

SEQ ID NO: 36 - DNA, 21 bp, Homo sapiens
  source 1..21: mol_type = unassigned DNA
tgaccttccacctgtttattg

SEQ ID NO: 37 - DNA, 21 bp, Homo sapiens
  source 1..21: mol_type = unassigned DNA
ccacctgtttattgctgcatt

SEQ ID NO: 38 - DNA, 21 bp, Homo sapiens
  source 1..21: mol_type = unassigned DNA
cctgtttattgctgcatttgt

SEQ ID NO: 39 - DNA, 21 bp, Homo sapiens
  source 1..21: mol_type = unassigned DNA
tacaactttgccgtccttaaa

SEQ ID NO: 40 - DNA, 21 bp, Homo sapiens
  source 1..21: mol_type = unassigned DNA
acaactttgccgtccttaaac

SEQ ID NO: 41 - skipped

SEQ ID NO: 42 - DNA, 21 bp, Homo sapiens
  source 1..21: mol_type = unassigned DNA
agatactcatagtcttggtag

SEQ ID NO: 43 - DNA, 21 bp, Homo sapiens
  source 1..21: mol_type = unassigned DNA
gatactcatagtcttggtagt

SEQ ID NO: 44 - DNA, 21 bp, Homo sapiens
  source 1..21: mol_type = unassigned DNA
gatgagatactcatagtcttg

SEQ ID NO: 45 - DNA, 21 bp, Homo sapiens
  source 1..21: mol_type = unassigned DNA
attgatgagatactcatagtc

SEQ ID NO: 46 - DNA, 21 bp, Homo sapiens
  source 1..21: mol_type = unassigned DNA
cattgatgagatactcatagt

SEQ ID NO: 47 - DNA, 21 bp, Homo sapiens
  source 1..21: mol_type = unassigned DNA
gatcacattgatgagatactc

SEQ ID NO: 48 - DNA, 21 bp, Homo sapiens
  source 1..21: mol_type = unassigned DNA
catactggaaggcatggatca

SEQ ID NO: 49 - DNA, 21 bp, Homo sapiens
  source 1..21: mol_type = unassigned DNA
catagatgacatactggaagg

SEQ ID NO: 50 - DNA, 21 bp, Homo sapiens
  source 1..21: mol_type = unassigned DNA
agttccatagatgacatactg

SEQ ID NO: 51 - DNA, 21 bp, Homo sapiens
  source 1..21: mol_type = unassigned DNA
gaaagaggcagttccatagat

SEQ ID NO: 52 - DNA, 21 bp, Homo sapiens
  source 1..21: mol_type = unassigned DNA
cataaaggaagaagaaagagg

SEQ ID NO: 53 - DNA, 21 bp, Homo sapiens
  source 1..21: mol_type = unassigned DNA
taaaggaagaagaaagaggca

SEQ ID NO: 54 - DNA, 21 bp, Homo sapiens
  source 1..21: mol_type = unassigned DNA
ataaaggaagaagaaagaggc

SEQ ID NO: 55 - DNA, 21 bp, Homo sapiens
  source 1..21: mol_type = unassigned DNA
cataaaggaagaagaaagagg

SEQ ID NO: 56 - DNA, 21 bp, Homo sapiens
  source 1..21: mol_type = unassigned DNA
gatggtggtcttgtagtcgcc

SEQ ID NO: 57 - DNA, 21 bp, Homo sapiens
  source 1..21: mol_type = unassigned DNA
agatggtggtcttgtagtcgc

SEQ ID NO: 58 - DNA, 21 bp, Homo sapiens
  source 1..21: mol_type = unassigned DNA
gaatgagcttgatgttggcct

SEQ ID NO: 59 - DNA, 21 bp, Homo sapiens
  source 1..21: mol_type = unassigned DNA
caaagaatgagcttgatgttg

SEQ ID NO: 60 - DNA, 21 bp, Homo sapiens
  source 1..21: mol_type = unassigned DNA
caatgacacacccgctccaaa

SEQ ID NO: 61 - DNA, 21 bp, Homo sapiens
  source 1..21: mol_type = unassigned DNA
caaacaatgacacacccgctc

SEQ ID NO: 62 - DNA, 21 bp, Homo sapiens
  source 1..21: mol_type = unassigned DNA
tttcccaaacaatgacacacc

SEQ ID NO: 63 - DNA, 21 bp, Homo sapiens
  source 1..21: mol_type = unassigned DNA
acttgtcgggatgtcctagcc

SEQ ID NO: 64 - DNA, 21 bp, Homo sapiens
  source 1..21: mol_type = unassigned DNA
acaaacttgtcgggatgtcct

SEQ ID NO: 65 - DNA, 21 bp, Homo sapiens
  source 1..21: mol_type = unassigned DNA
cataggtgatgcccacaaact

SEQ ID NO: 66 - DNA, 21 bp, Homo sapiens
  source 1..21: mol_type = unassigned DNA
tgtacacaggcacagcagagc

SEQ ID NO: 67 - DNA, 21 bp, Homo sapiens
  source 1..21: mol_type = unassigned DNA
gtaaatgtacacaggcacagc

SEQ ID NO: 68 - DNA, 21 bp, Homo sapiens
  source 1..21: mol_type = unassigned DNA
aaatgtacacaggcacagcag

SEQ ID NO: 69 - DNA, 21 bp, Homo sapiens
  source 1..21: mol_type = unassigned DNA
agtaaatgtacacaggcacag

SEQ ID NO: 70 - DNA, 21 bp, Homo sapiens
  source 1..21: mol_type = unassigned DNA
taaatgtacacaggcacagca

SEQ ID NO: 71 - DNA, 21 bp, Homo sapiens
  source 1..21: mol_type = unassigned DNA
gtaaatgtacacaggcacagc

SEQ ID NO: 72 - DNA, 21 bp, Homo sapiens
  source 1..21: mol_type = unassigned DNA
tgttgaagtaaatgtacacag

SEQ ID NO: 73 - DNA, 21 bp, Homo sapiens
  source 1..21: mol_type = unassigned DNA
aatagactggcaggtggtcca

SEQ ID NO: 74 - DNA, 21 bp, Homo sapiens
  source 1..21: mol_type = unassigned DNA
tatactggcagaggtcttgct

SEQ ID NO: 75 - DNA, 21 bp, Homo sapiens
  source 1..21: mol_type = unassigned DNA
aataaacaggtggaaggtcat

SEQ ID NO: 76 - DNA, 21 bp, Homo sapiens
  source 1..21: mol_type = unassigned DNA
caataaacaggtggaaggtca

SEQ ID NO: 77 - DNA, 21 bp, Homo sapiens
  source 1..21: mol_type = unassigned DNA
aatgcagcaataaacaggtgg

SEQ ID NO: 78 - DNA, 21 bp, Homo sapiens
  source 1..21: mol_type = unassigned DNA
acaaatgcagcaataaacagg

SEQ ID NO: 79 - DNA, 21 bp, Homo sapiens
  source 1..21: mol_type = unassigned DNA
tttaaggacggcaaagttgta

SEQ ID NO: 80 - DNA, 21 bp, Homo sapiens
  source 1..21: mol_type = unassigned DNA
gtttaaggacggcaaagttgt

SEQ ID NO: 81 - DNA, 423 bp, Homo sapiens
  source 1..423: mol_type = unassigned DNA
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 82 - DNA, 21 bp, Homo sapiens
  source 1..21: mol_type = unassigned DNA
ggatgtattcatgaaaggact

SEQ ID NO: 83 - DNA, 21 bp, Homo sapiens
  source 1..21: mol_type = unassigned DNA
atgtattcatgaaaggacttt

SEQ ID NO: 84 - DNA, 21 bp, Homo sapiens
  source 1..21: mol_type = unassigned DNA
tgtattcatgaaaggactttc

SEQ ID NO: 85 - DNA, 21 bp, Homo sapiens
  source 1..21: mol_type = unassigned DNA
agttgtggctgctgctgagaa

SEQ ID NO: 86 - DNA, 21 bp, Homo sapiens
  source 1..21: mol_type = unassigned DNA
gttgtggctgctgctgagaaa

SEQ ID NO: 87 - DNA, 21 bp, Homo sapiens
  source 1..21: mol_type = unassigned DNA
tgtggcagaagcagcaggaaa

SEQ ID NO: 88 - DNA, 21 bp, Homo sapiens
  source 1..21: mol_type = unassigned DNA
tggcagaagcagcaggaaaga

SEQ ID NO: 89 - DNA, 21 bp, Homo sapiens
  source 1..21: mol_type = unassigned DNA
agagggtgttctctatgtagg

SEQ ID NO: 90 - DNA, 21 bp, Homo sapiens
  source 1..21: mol_type = unassigned DNA
cagtggctgagaagaccaaag

SEQ ID NO: 91 - DNA, 21 bp, Homo sapiens
  source 1..21: mol_type = unassigned DNA
gaccaaagagcaagtgacaaa

SEQ ID NO: 92 - DNA, 21 bp, Homo sapiens
  source 1..21: mol_type = unassigned DNA
ccaaagagcaagtgacaaatg

SEQ ID NO: 93 - DNA, 21 bp, Homo sapiens
  source 1..21: mol_type = unassigned DNA
caaagagcaagtgacaaatgt

SEQ ID NO: 94 - DNA, 21 bp, Homo sapiens
  source 1..21: mol_type = unassigned DNA
accaaagagcaagtgacaaat

SEQ ID NO: 95 - DNA, 21 bp, Homo sapiens
  source 1..21: mol_type = unassigned DNA
ccaaagagcaagtgacaaatg

SEQ ID NO: 96 - DNA, 21 bp, Homo sapiens
  source 1..21: mol_type = unassigned DNA
caaagagcaagtgacaaatgt

SEQ ID NO: 97 - DNA, 21 bp, Homo sapiens
  source 1..21: mol_type = unassigned DNA
agagcaagtgacaaatgttgg

SEQ ID NO: 98 - DNA, 21 bp, Homo sapiens
  source 1..21: mol_type = unassigned DNA
gcaagtgacaaatgttggagg

SEQ ID NO: 99 - DNA, 21 bp, Homo sapiens
  source 1..21: mol_type = unassigned DNA
agtgacaaatgttggaggagc

SEQ ID NO: 100 - DNA, 21 bp, Homo sapiens
  source 1..21: mol_type = unassigned DNA
ggaccagttgggcaagaatga

SEQ ID NO: 101 - DNA, 21 bp, Homo sapiens
  source 1..21: mol_type = unassigned DNA
aggaccagttgggcaagaatg

SEQ ID NO: 102 - DNA, 21 bp, Homo sapiens
  source 1..21: mol_type = unassigned DNA
gaccagttgggcaagaatgaa

SEQ ID NO: 103 - DNA, 21 bp, Homo sapiens
  source 1..21: mol_type = unassigned DNA
gttgggcaagaatgaagaagg

SEQ ID NO: 104 - DNA, 21 bp, Homo sapiens
  source 1..21: mol_type = unassigned DNA
gggcaagaatgaagaaggagc

SEQ ID NO: 105 - DNA, 21 bp, Homo sapiens
  source 1..21: mol_type = unassigned DNA
ggaaggaattctggaagatat

SEQ ID NO: 106 - DNA, 21 bp, Homo sapiens
  source 1..21: mol_type = unassigned DNA
gaaggaattctggaagatatg

SEQ ID NO: 107 - DNA, 21 bp, Homo sapiens
  source 1..21: mol_type = unassigned DNA
ggaagatatgcctgtggatcc

SEQ ID NO: 108 - DNA, 21 bp, Homo sapiens
  source 1..21: mol_type = unassigned DNA
gcctgtggatcctgacaatga

SEQ ID NO: 109 - DNA, 21 bp, Homo sapiens
  source 1..21: mol_type = unassigned DNA
tgcctgtggatcctgacaatg

SEQ ID NO: 110 - DNA, 21 bp, Homo sapiens
  source 1..21: mol_type = unassigned DNA
gatcctgacaatgaggcttat

SEQ ID NO: 111 - DNA, 21 bp, Homo sapiens
  source 1..21: mol_type = unassigned DNA
atcctgacaatgaggcttatg

SEQ ID NO: 112 - DNA, 21 bp, Homo sapiens
  source 1..21: mol_type = unassigned DNA
tcctgacaatgaggcttatga

SEQ ID NO: 113 - DNA, 21 bp, Homo sapiens
  source 1..21: mol_type = unassigned DNA
cctgacaatgaggcttatgaa

SEQ ID NO: 114 - DNA, 21 bp, Homo sapiens
  source 1..21: mol_type = unassigned DNA
gacaatgaggcttatgaaatg

SEQ ID NO: 115 - DNA, 21 bp, Homo sapiens
  source 1..21: mol_type = unassigned DNA
ctgacaatgaggcttatgaaa

SEQ ID NO: 116 - DNA, 21 bp, Homo sapiens
  source 1..21: mol_type = unassigned DNA
tgacaatgaggcttatgaaat

SEQ ID NO: 117 - DNA, 21 bp, Homo sapiens
  source 1..21: mol_type = unassigned DNA
gacaatgaggcttatgaaatg

SEQ ID NO: 118 - DNA, 21 bp, Homo sapiens
  source 1..21: mol_type = unassigned DNA
aggaagggtatcaagactacg

SEQ ID NO: 119 - DNA, 21 bp, Homo sapiens
  source 1..21: mol_type = unassigned DNA
agggtatcaagactacgaacc

SEQ ID NO: 120 - DNA, 21 bp, Homo sapiens
  source 1..21: mol_type = unassigned DNA
tcaagactacgaacctgaagc

SEQ ID NO: 121 - DNA, 19 bp, Homo sapiens
  source 1..19: mol_type = unassigned DNA
ctacgaacctgaagcctaa

SEQ ID NO: 122 - DNA, 21 bp, Homo sapiens
  source 1..21: mol_type = unassigned DNA
agtcctttcatgaatacatcc

SEQ ID NO: 123 - DNA, 21 bp, Homo sapiens
  source 1..21: mol_type = unassigned DNA
aaagtcctttcatgaatacat

SEQ ID NO: 124 - DNA, 21 bp, Homo sapiens
  source 1..21: mol_type = unassigned DNA
gaaagtcctttcatgaataca

SEQ ID NO: 125 - DNA, 21 bp, Homo sapiens
  source 1..21: mol_type = unassigned DNA
ttctcagcagcagccacaact

SEQ ID NO: 126 - DNA, 21 bp, Homo sapiens
  source 1..21: mol_type = unassigned DNA
tttctcagcagcagccacaac

SEQ ID NO: 127 - DNA, 21 bp, Homo sapiens
  source 1..21: mol_type = unassigned DNA
tttcctgctgcttctgccaca

SEQ ID NO: 128 - DNA, 21 bp, Homo sapiens
  source 1..21: mol_type = unassigned DNA
tctttcctgctgcttctgcca

SEQ ID NO: 129 - DNA, 21 bp, Homo sapiens
  source 1..21: mol_type = unassigned DNA
cctacatagagaacaccctct

SEQ ID NO: 130 - DNA, 21 bp, Homo sapiens
  source 1..21: mol_type = unassigned DNA
ctttggtcttctcagccactg

SEQ ID NO: 131 - DNA, 21 bp, Homo sapiens
  source 1..21: mol_type = unassigned DNA
tttgtcacttgctctttggtc

SEQ ID NO: 132 - DNA, 21 bp, Homo sapiens
  source 1..21: mol_type = unassigned DNA
catttgtcacttgctctttgg

SEQ ID NO: 133 - DNA, 21 bp, Homo sapiens
  source 1..21: mol_type = unassigned DNA
acatttgtcacttgctctttg

SEQ ID NO: 134 - DNA, 21 bp, Homo sapiens
  source 1..21: mol_type = unassigned DNA
atttgtcacttgctctttggt

SEQ ID NO: 135 - DNA, 21 bp, Homo sapiens
  source 1..21: mol_type = unassigned DNA
catttgtcacttgctctttgg

SEQ ID NO: 136 - DNA, 21 bp, Homo sapiens
  source 1..21: mol_type = unassigned DNA
acatttgtcacttgctctttg

SEQ ID NO: 137 - DNA, 21 bp, Homo sapiens
  source 1..21: mol_type = unassigned DNA
ccaacatttgtcacttgctct

SEQ ID NO: 138 - DNA, 21 bp, Homo sapiens
  source 1..21: mol_type = unassigned DNA
cctccaacatttgtcacttgc

SEQ ID NO: 139 - DNA, 21 bp, Homo sapiens
  source 1..21: mol_type = unassigned DNA
gctcctccaacatttgtcact

SEQ ID NO: 140 - DNA, 21 bp, Homo sapiens
  source 1..21: mol_type = unassigned DNA
tcattcttgcccaactggtcc

SEQ ID NO: 141 - DNA, 21 bp, Homo sapiens
  source 1..21: mol_type = unassigned DNA
cattcttgcccaactggtcct

SEQ ID NO: 142 - DNA, 21 bp, Homo sapiens
  source 1..21: mol_type = unassigned DNA
ttcattcttgcccaactggtc

SEQ ID NO: 143 - DNA, 21 bp, Homo sapiens
  source 1..21: mol_type = unassigned DNA
ccttcttcattcttgcccaac

SEQ ID NO: 144 - DNA, 21 bp, Homo sapiens
  source 1..21: mol_type = unassigned DNA
gctccttcttcattcttgccc

SEQ ID NO: 145 - DNA, 21 bp, Homo sapiens
  source 1..21: mol_type = unassigned DNA
atatcttccagaattccttcc

SEQ ID NO: 146 - DNA, 21 bp, Homo sapiens
  source 1..21: mol_type = unassigned DNA
catatcttccagaattccttc

SEQ ID NO: 147 - DNA, 21 bp, Homo sapiens
  source 1..21: mol_type = unassigned DNA
ggatccacaggcatatcttcc

SEQ ID NO: 148 - DNA, 21 bp, Homo sapiens
  source 1..21: mol_type = unassigned DNA
tcattgtcaggatccacaggc

SEQ ID NO: 149 - DNA, 21 bp, Homo sapiens
  source 1..21: mol_type = unassigned DNA
cattgtcaggatccacaggca

SEQ ID NO: 150 - DNA, 21 bp, Homo sapiens
  source 1..21: mol_type = unassigned DNA
ataagcctcattgtcaggatc

SEQ ID NO: 151 - DNA, 21 bp, Homo sapiens
  source 1..21: mol_type = unassigned DNA
cataagcctcattgtcaggat

SEQ ID NO: 152 - DNA, 21 bp, Homo sapiens
  source 1..21: mol_type = unassigned DNA
tcataagcctcattgtcagga

SEQ ID NO: 153 - DNA, 21 bp, Homo sapiens
  source 1..21: mol_type = unassigned DNA
ttcataagcctcattgtcagg

SEQ ID NO: 154 - DNA, 21 bp, Homo sapiens
  source 1..21: mol_type = unassigned DNA
catttcataagcctcattgtc

SEQ ID NO: 155 - DNA, 21 bp, Homo sapiens
  source 1..21: mol_type = unassigned DNA
tttcataagcctcattgtcag

SEQ ID NO: 156 - DNA, 21 bp, Homo sapiens
  source 1..21: mol_type = unassigned DNA
atttcataagcctcattgtca

SEQ ID NO: 157 - DNA, 21 bp, Homo sapiens
  source 1..21: mol_type = unassigned DNA
catttcataagcctcattgtc

SEQ ID NO: 158 - DNA, 21 bp, Homo sapiens
  source 1..21: mol_type = unassigned DNA
cgtagtcttgatacccttcct

SEQ ID NO: 159 - DNA, 21 bp, Homo sapiens
  source 1..21: mol_type = unassigned DNA
ggttcgtagtcttgataccct

SEQ ID NO: 160 - DNA, 21 bp, Homo sapiens
  source 1..21: mol_type = unassigned DNA
gcttcaggttcgtagtcttga

SEQ ID NO: 161 - DNA, 19 bp, Homo sapiens
  source 1..19: mol_type = unassigned DNA
ttaggcttcaggttcgtag

SEQ ID NO: 162 - DNA, 1488 bp, Homo sapiens
  source 1..1488: mol_type = unassigned DNA
atgagaaggggggctgcggaccgagaaactgagcggctcccgggggcgcagggaccgtgctccgccgtctccgccgcatcttccaccctcgccgccgccgcagctccccgcgctcgtgccaccgccgccgcgtccaccctcagcgccaccgccatgcgggagatcgtgcacctgcaggccggccagtgcggcaaccagatcggggccaagttttgggaggttatcagtgacgaacatggcatcgaccccacaggcacataccatggggacagtgacctgcaactggagaggatcaacgtgtactacaacgaggccacaggaggaaattatgtccccagagcggtgctggtggacctggaacccggcaccatggactctgtccgttctggccccttcggtcagatctttcggccggacaacttcgtgtttggccaatccggagccggcaacaactgggcaaaggggcactacacggagggcgcagagctggtggacgctgtcctggacgtagtccggaaggaggccgagagctgcgactgccttcagggcttccagctgacccactcgctggggggtggcacggggtccggaatgggcacgctgctcatcagtaagatccgcgaggagttcccagaccgcatcatgaacaccttcagcgtggtgccctcgcccaaagtgtcagacacggtggtggagccctacaacgccacgctgtctgtgcaccagctggtggagaatacggatgagacctactgcatcgacaacgaggcactctacgacatctgtttccgcaccctcaagctgaccacccccacctacggggacctcaaccacctggtgtcggccaccatgagcggggtcaccacctgcctgcgcttcccgggccagctgaacgccgacctgcgcaagctggccgtcaacatggttccctttcctcgcctgcacttcttcatgcccggcttcgcacccctgaccagccggggcagccagcagtaccgggccctgacggtgcccgagctcacccagcagatgttcgatgccaagaacatgatggcggcgtgcgacccgcgccacggccgctacctgaccgtggccgccgtgttccggggccgcatgtccatgaaggaggtggacgagcagatgctgagcgtgcagagcaagaacagcagctacttcgtggagtggatccccaacaacgtgaagacggccgtgtgcgacatcccgccccgcggcctgaagatggccgcgaccttcatcggcaacagcacggccatccaggagctgttcaagcgcatctccgagcagttcacggccatgttccggcgcaaggccttcttgcactggtacacgggcgagggcatggacgagatggagttcaccgaggccgagagcaacatgaatgacctggtatctgagtaccagcagtaccaggacgccacggccgaggagggcgagttcgaggaggaggcggaggaggaggtggcctag

SEQ ID NO: 163 - DNA, 21 bp, Homo sapiens
  source 1..21: mol_type = unassigned DNA
gggaggttatcagtgacgaac

SEQ ID NO: 164 - DNA, 21 bp, Homo sapiens
  source 1..21: mol_type = unassigned DNA
ggaggttatcagtgacgaaca

SEQ ID NO: 165 - DNA, 21 bp, Homo sapiens
  source 1..21: mol_type = unassigned DNA
ggttatcagtgacgaacatgg

SEQ ID NO: 166 - DNA, 21 bp, Homo sapiens
  source 1..21: mol_type = unassigned DNA
gaggttatcagtgacgaacat

SEQ ID NO: 167 - DNA, 21 bp, Homo sapiens
  source 1..21: mol_type = unassigned DNA
aggttatcagtgacgaacatg

SEQ ID NO: 168 - DNA, 21 bp, Homo sapiens
  source 1..21: mol_type = unassigned DNA
cctgcaactggagaggatcaa

SEQ ID NO: 169 - DNA, 21 bp, Homo sapiens
  source 1..21: mol_type = unassigned DNA
ctgcaactggagaggatcaac

SEQ ID NO: 170 - DNA, 21 bp, Homo sapiens
  source 1..21: mol_type = unassigned DNA
tggagaggatcaacgtgtact

SEQ ID NO: 171 - DNA, 21 bp, Homo sapiens
  source 1..21: mol_type = unassigned DNA
ggagaggatcaacgtgtacta

SEQ ID NO: 172 - DNA, 21 bp, Homo sapiens
  source 1..21: mol_type = unassigned DNA
gagaggatcaacgtgtactac

SEQ ID NO: 173 - DNA, 21 bp, Homo sapiens
  source 1..21: mol_type = unassigned DNA
agaggatcaacgtgtactaca

SEQ ID NO: 174 - DNA, 21 bp, Homo sapiens
  source 1..21: mol_type = unassigned DNA
ggatcaacgtgtactacaacg

SEQ ID NO: 175 - DNA, 21 bp, Homo sapiens
  source 1..21: mol_type = unassigned DNA
gaggatcaacgtgtactacaa

SEQ ID NO: 176 - DNA, 21 bp, Homo sapiens
  source 1..21: mol_type = unassigned DNA
acgaggccacaggaggaaatt

SEQ ID NO: 177 - DNA, 21 bp, Homo sapiens
  source 1..21: mol_type = unassigned DNA
cgaggccacaggaggaaatta

SEQ ID NO: 178 - DNA, 21 bp, Homo sapiens
  source 1..21: mol_type = unassigned DNA
gaggccacaggaggaaattat

SEQ ID NO: 179 - DNA, 21 bp, Homo sapiens
  source 1..21: mol_type = unassigned DNA
aggccacaggaggaaattatg

SEQ ID NO: 180 - DNA, 21 bp, Homo sapiens
  source 1..21: mol_type = unassigned DNA
ggccacaggaggaaattatgt

SEQ ID NO: 181 - DNA, 21 bp, Homo sapiens
  source 1..21: mol_type = unassigned DNA
ccagagcggtgctggtggacc

SEQ ID NO: 182 - DNA, 21 bp, Homo sapiens
  source 1..21: mol_type = unassigned DNA
ggtgctggtggacctggaacc

SEQ ID NO: 183 - DNA, 21 bp, Homo sapiens
  source 1..21: mol_type = unassigned DNA
agatctttcggccggacaact

SEQ ID NO: 184 - DNA, 21 bp, Homo sapiens
  source 1..21: mol_type = unassigned DNA
caacttcgtgtttggccaatc

SEQ ID NO: 185 - DNA, 21 bp, Homo sapiens
  source 1..21: mol_type = unassigned DNA
gcacgctgctcatcagtaaga

SEQ ID NO: 186 - DNA, 21 bp, Homo sapiens
  source 1..21: mol_type = unassigned DNA
cacgctgctcatcagtaagat

SEQ ID NO: 187 - DNA, 21 bp, Homo sapiens
  source 1..21: mol_type = unassigned DNA
cggtggtggagccctacaacg

SEQ ID NO: 188 - DNA, 21 bp, Homo sapiens
  source 1..21: mol_type = unassigned DNA
tgcaccagctggtggagaata

SEQ ID NO: 189 - DNA, 21 bp, Homo sapiens
  source 1..21: mol_type = unassigned DNA
gctggtggagaatacggatga

SEQ ID NO: 190 - DNA, 21 bp, Homo sapiens
  source 1..21: mol_type = unassigned DNA
cggatgagacctactgcatcg

SEQ ID NO: 191 - DNA, 21 bp, Homo sapiens
  source 1..21: mol_type = unassigned DNA
gagacctactgcatcgacaac

SEQ ID NO: 192 - DNA, 21 bp, Homo sapiens
  source 1..21: mol_type = unassigned DNA
agacctactgcatcgacaacg

SEQ ID NO: 193 - DNA, 21 bp, Homo sapiens
  source 1..21: mol_type = unassigned DNA
cctactgcatcgacaacgagg

SEQ ID NO: 194 - DNA, 21 bp, Homo sapiens
  source 1..21: mol_type = unassigned DNA
tcgacaacgaggcactctacg

SEQ ID NO: 195 - DNA, 21 bp, Homo sapiens
  source 1..21: mol_type = unassigned DNA
acgaggcactctacgacatct

SEQ ID NO: 196 - DNA, 21 bp, Homo sapiens
  source 1..21: mol_type = unassigned DNA
gcactctacgacatctgtttc

SEQ ID NO: 197 - DNA, 21 bp, Homo sapiens
  source 1..21: mol_type = unassigned DNA
ccgtcaacatggttccctttc

SEQ ID NO: 198 - DNA, 21 bp, Homo sapiens
  source 1..21: mol_type = unassigned DNA
gctacttcgtggagtggatcc

SEQ ID NO: 199 - DNA, 21 bp, Homo sapiens
  source 1..21: mol_type = unassigned DNA
caggagctgttcaagcgcatc

SEQ ID NO: 200 - DNA, 21 bp, Homo sapiens
  source 1..21: mol_type = unassigned DNA
aggccttcttgcactggtaca

SEQ ID NO: 201 - DNA, 21 bp, Homo sapiens
  source 1..21: mol_type = unassigned DNA
gaggccgagagcaacatgaat

SEQ ID NO: 202 - DNA, 21 bp, Homo sapiens
  source 1..21: mol_type = unassigned DNA
aggccgagagcaacatgaatg

SEQ ID NO: 203 - DNA, 21 bp, Homo sapiens
  source 1..21: mol_type = unassigned DNA
caacatgaatgacctggtatc

SEQ ID NO: 204 - DNA, 21 bp, Homo sapiens
  source 1..21: mol_type = unassigned DNA
gttcgtcactgataacctccc

SEQ ID NO: 205 - DNA, 21 bp, Homo sapiens
  source 1..21: mol_type = unassigned DNA
tgttcgtcactgataacctcc

SEQ ID NO: 206 - DNA, 21 bp, Homo sapiens
  source 1..21: mol_type = unassigned DNA
ccatgttcgtcactgataacc

SEQ ID NO: 207 - DNA, 21 bp, Homo sapiens
  source 1..21: mol_type = unassigned DNA
atgttcgtcactgataacctc

SEQ ID NO: 208 - DNA, 21 bp, Homo sapiens
  source 1..21: mol_type = unassigned DNA
catgttcgtcactgataacct

SEQ ID NO: 209 - DNA, 21 bp, Homo sapiens
  source 1..21: mol_type = unassigned DNA
ttgatcctctccagttgcagg

SEQ ID NO: 210 - DNA, 21 bp, Homo sapiens
  source 1..21: mol_type = unassigned DNA
gttgatcctctccagttgcag

SEQ ID NO: 211 - DNA, 21 bp, Homo sapiens
  source 1..21: mol_type = unassigned DNA
agtacacgttgatcctctcca

SEQ ID NO: 212 - DNA, 21 bp, Homo sapiens
  source 1..21: mol_type = unassigned DNA
tagtacacgttgatcctctcc

SEQ ID NO: 213 - DNA, 21 bp, Homo sapiens
  source 1..21: mol_type = unassigned DNA
gtagtacacgttgatcctctc

SEQ ID NO: 214 - DNA, 21 bp, Homo sapiens
  source 1..21: mol_type = unassigned DNA
tgtagtacacgttgatcctct

SEQ ID NO: 215 - DNA, 21 bp, Homo sapiens
  source 1..21: mol_type = unassigned DNA
cgttgtagtacacgttgatcc

SEQ ID NO: 216 - DNA, 21 bp, Homo sapiens
  source 1..21: mol_type = unassigned DNA
ttgtagtacacgttgatcctc

SEQ ID NO: 217 - DNA, 21 bp, Homo sapiens
  source 1..21: mol_type = unassigned DNA
aatttcctcctgtggcctcgt

SEQ ID NO: 218 - DNA, 21 bp, Homo sapiens
  source 1..21: mol_type = unassigned DNA
taatttcctcctgtggcctcg

SEQ ID NO: 219 - DNA, 21 bp, Homo sapiens
  source 1..21: mol_type = unassigned DNA
ataatttcctcctgtggcctc

SEQ ID NO: 220 - DNA, 21 bp, Homo sapiens
  source 1..21: mol_type = unassigned DNA
cataatttcctcctgtggcct

SEQ ID NO: 221 - DNA, 21 bp, Homo sapiens
  source 1..21: mol_type = unassigned DNA
acataatttcctcctgtggcc

SEQ ID NO: 222 - DNA, 21 bp, Homo sapiens
  source 1..21: mol_type = unassigned DNA
ggtccaccagcaccgctctgg

SEQ ID NO: 223 - DNA, 21 bp, Homo sapiens
  source 1..21: mol_type = unassigned DNA
ggttccaggtccaccagcacc

SEQ ID NO: 224 - DNA, 21 bp, Homo sapiens
  source 1..21: mol_type = unassigned DNA
agttgtccggccgaaagatct

SEQ ID NO: 225 - DNA, 21 bp, Homo sapiens
  source 1..21: mol_type = unassigned DNA
gattggccaaacacgaagttg

SEQ ID NO: 226 - DNA, 21 bp, Homo sapiens
  source 1..21: mol_type = unassigned DNA
tcttactgatgagcagcgtgc

SEQ ID NO: 227 - DNA, 21 bp, Homo sapiens
  source 1..21: mol_type = unassigned DNA
atcttactgatgagcagcgtg

SEQ ID NO: 228 - DNA, 21 bp, Homo sapiens
  source 1..21: mol_type = unassigned DNA
cgttgtagggctccaccaccg

SEQ ID NO: 229 - DNA, 21 bp, Homo sapiens
  source 1..21: mol_type = unassigned DNA
tattctccaccagctggtgca

SEQ ID NO: 230 - DNA, 21 bp, Homo sapiens
  source 1..21: mol_type = unassigned DNA
tcatccgtattctccaccagc

SEQ ID NO: 231 - DNA, 21 bp, Homo sapiens
  source 1..21: mol_type = unassigned DNA
cgatgcagtaggtctcatccg

SEQ ID NO: 232 - DNA, 21 bp, Homo sapiens
  source 1..21: mol_type = unassigned DNA
gttgtcgatgcagtaggtctc

SEQ ID NO: 233 - DNA, 21 bp, Homo sapiens
  source 1..21: mol_type = unassigned DNA
cgttgtcgatgcagtaggtct

SEQ ID NO: 234 - DNA, 21 bp, Homo sapiens
  source 1..21: mol_type = unassigned DNA
cctcgttgtcgatgcagtagg

SEQ ID NO: 235 - DNA, 21 bp, Homo sapiens
  source 1..21: mol_type = unassigned DNA
cgtagagtgcctcgttgtcga

SEQ ID NO: 236 - DNA, 21 bp, Homo sapiens
  source 1..21: mol_type = unassigned DNA
agatgtcgtagagtgcctcgt

SEQ ID NO: 237 - DNA, 21 bp, Homo sapiens
  source 1..21: mol_type = unassigned DNA
gaaacagatgtcgtagagtgc

SEQ ID NO: 238 - DNA, 21 bp, Homo sapiens
  source 1..21: mol_type = unassigned DNA
gaaagggaaccatgttgacgg

SEQ ID NO: 239 - DNA, 21 bp, Homo sapiens
  source 1..21: mol_type = unassigned DNA
ggatccactccacgaagtagc

SEQ ID NO: 240 - DNA, 21 bp, Homo sapiens
  source 1..21: mol_type = unassigned DNA
gatgcgcttgaacagctcctg

SEQ ID NO: 241 - DNA, 21 bp, Homo sapiens
  source 1..21: mol_type = unassigned DNA
tgtaccagtgcaagaaggcct

SEQ ID NO: 242 - DNA, 21 bp, Homo sapiens
  source 1..21: mol_type = unassigned DNA
attcatgttgctctcggcctc

SEQ ID NO: 243 - DNA, 21 bp, Homo sapiens
  source 1..21: mol_type = unassigned DNA
cattcatgttgctctcggcct

SEQ ID NO: 244 - DNA, 21 bp, Homo sapiens
  source 1..21: mol_type = unassigned DNA
gataccaggtcattcatgttg

SEQ ID NO: 245 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEQSPQEPDSSSGIGKTGQQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTVQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 246 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTDNNGVKTIANNLTSTVQVFTDSDYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 247 - protein, 736 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLQGDTSFGGNLGRAVFQAKKRVLEPLGLVEEGAKTAPGKKRPVEQSPQEPDSSSGIGETGQQPAKKRLNFGQTGDSESVPDPQPLGEPPATPAAVGPTTMASGGGAPMADNNEGADGVGSSSGNWHCDSQWLGDRVITTSTRTWALPTYNNHLYKQISSASTGASNDNHYFGYSTPWGYFDFNRFHCHFSPRDWQRLINNNWGFRPKRLSFKLFNIQVKEVTDNNGVKTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDKERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPHPIGTRYLTRPL

SEQ ID NO: 248 - DNA, 116 bp, Homo sapiens
  source 1..116: mol_type = unassigned DNA
aatggtttgtggattttgaaaaggtgcaggccatattgtgctgcctcaaaaatacaaggatctgatcttctgaagaaaatatatttctttttattcatagctcttatgatagcaat

SEQ ID NO: 249 - RNA, 89 nt, synthetic construct
  source 1..89: mol_type = other RNA
gtcagcagtgccttagcagcacgtaaatattggcgttaagattctaaaattatctccagtattaactgtgctgctgaagtaaggttgac

SEQ ID NO: 250 - DNA, 115 bp, Homo sapiens
  source 1..115: mol_type = unassigned DNA
catactctacagttgtgttttaatgtatattaatgttactaatgtgttttcagttttattgatagtcttttcagtattattgataatcttgttatttttagtatgattctgtaaa

SEQ ID NO: 251 - DNA, 92 bp, Homo sapiens
  source 1..92: mol_type = unassigned DNA
ggtttgtttttgtttttgtttttttatcaaatcctgcctgactgtctgcttgttttgcctaccatcgtgacatctccatggctgtaccacct

SEQ ID NO: 252 - DNA, 72 bp, Homo sapiens
  source 1..72: mol_type = unassigned DNA
tgtcgggtagcttatcagactgatgttgactgttgaatctcatggcaacaccagtcgatgggctgtctgaca

SEQ ID NO: 253 - RNA, 72 nt, synthetic construct
  source 1..72: mol_type = other RNA
tgtcgggtagcttatcagactgatgttgactgttgaatctcatggcaacaccagtcgatgggctgtctgaca

SEQ ID NO: 254 - DNA, 95 bp, Homo sapiens
  source 1..95: mol_type = unassigned DNA
ttttggtatctttcatctgaccatccatatccaatgttctcatttaaacattacccagcatcattgtttataatcagaaactctggtccttctgt

SEQ ID NO: 255 - DNA, 95 bp, Homo sapiens
  source 1..95: mol_type = unassigned DNA
cccaaatctcattacctcctttgctctctctctctttctcccctccaggtgccagcctctggccccgcccggtgcccccctcacccctgtgccac

SEQ ID NO: 256 - RNA, 73 nt, synthetic construct
  source 1..73: mol_type = other RNA
ggccggctggggttcctggggatgggatttgcttcctgtcacaaatcacattgccagggatttccaaccgacc

SEQ ID NO: 257 - DNA, 95 bp, Homo sapiens
  source 1..95: mol_type = unassigned DNA
ctgagctctgccaccgaggatgctgcccggggacggggtggcagagaggccccgaagcctgtgcctggcctgaggagcagggcttagctgcttgt

SEQ ID NO: 258 - DNA, 93 bp, Homo sapiens
  source 1..93: mol_type = unassigned DNA
cgttgcctgcacatcttggaaacacttgctgggattacttcttcaggttaacccaacagaaggctaaagaaggtatattgctgttgacagtga

SEQ ID NO: 259 - DNA, 71 bp, Homo sapiens
  source 1..71: mol_type = unassigned DNA
gcgactgtaaacatcctcgactggaagctgtgaagccacagatgggctttcagtcggatgtttgcagctgc

SEQ ID NO: 260 - RNA, 71 nt, synthetic construct
  source 1..71: mol_type = other RNA
gcgactgtaaacatcctcgactggaagctgtgaagccacagatgggctttcagtcggatgtttgcagctgc

SEQ ID NO: 261 - DNA, 94 bp, Homo sapiens
  source 1..94: mol_type = unassigned DNA
ctactgcctcggacttcaaggggctactttaggagcaattatcttgtttactaaaactgaataccttgctatctctttgatacatttttacaaa

SEQ ID NO: 262 - DNA, 96 bp, Homo sapiens
  source 1..96: mol_type = unassigned DNA
aattaatgagtgtgttttccctccctcaggtgaaaggaaaaattttggaaaagtaaaacactgaagagtcatagtattctcctgtaacttggaact

SEQ ID NO: 263 - DNA, 71 bp, Homo sapiens
  source 1..71: mol_type = unassigned DNA
ggagaggaggcaagatgctggcatagctgttgaactgggaacctgctatgccaacatattgccatctttcc

SEQ ID NO: 264 - RNA, 71 nt, synthetic construct
  source 1..71: mol_type = other RNA
ggagaggaggcaagatgctggcatagctgttgaactgggaacctgctatgccaacatattgccatctttcc

SEQ ID NO: 265 - DNA, 93 bp, Homo sapiens
  source 1..93: mol_type = unassigned DNA
tgtctgacagcagccatggccacctgcatgccagtccttcgtgtattgctgtgtatgtgcgcccttccttggatgtggatttccatgacatgg

SEQ ID NO: 266 - DNA, 118 bp, Homo sapiens
  source 1..118: mol_type = unassigned DNA
cgtgatgcttcttttctctgcttaggtcacaatatgtggagctgacaaggttcccctattatcagtgacaatggtggaatgtggaggtgaagttaacaccttcgtggctacagagttt

SEQ ID NO: 267 - DNA, 85 bp, Homo sapiens
  source 1..85: mol_type = unassigned DNA
ccttagcagagctgtggagtgtgacaatggtgtttgtgtctaaactatcaaacgccattatcacactaaatagctactgctaggc

SEQ ID NO: 268 - RNA, 85 nt, synthetic construct
  source 1..85: mol_type = other RNA
ccttagcagagctgtggagtgtgacaatggtgtttgtgtctaaactatcaaacgccattatcacactaaatagctactgctaggc

SEQ ID NO: 269 - DNA, 117 bp, Homo sapiens
  source 1..117: mol_type = unassigned DNA
aatccttccctcgataaatgtcttggcatcgtttgctttgagcaagaaggttcatctgatatcagtcttctcaatcttgtgtacttactgaataaagtctggctcttttgcactcat

SEQ ID NO: 270 - DNA, 87 bp, Homo sapiens
  source 1..87: mol_type = unassigned DNA
gtggcacaaaccaggaaggggaaatctgtggtttaaattctttatgcctcatcctctgagtgctgaaggcttgctgtaggctgtatg

SEQ ID NO: 271 - DNA, 65 bp, Homo sapiens
  source 1..65: mol_type = unassigned DNA
ctgttaatgctaatcgtgataggggtttttgcctccaactgactcctacatattagcattaacag

SEQ ID NO: 272 - RNA, 65 nt, synthetic construct
  source 1..65: mol_type = other RNA
ctgttaatgctaatcgtgataggggtttttgcctccaactgactcctacatattagcattaacag

SEQ ID NO: 273 - DNA, 87 bp, Homo sapiens
  source 1..87: mol_type = unassigned DNA
tgtatgatgcctgttactagcattcacatggaacaaattgctgccgtgggaggatgacaaagaagcatgagtcaccctgctggataa

SEQ ID NO: 274 - DNA, 96 bp, Homo sapiens
  source 1..96: mol_type = unassigned DNA
ccccagctctggagcctgacaaggaggacaggagagatgctgcaagcccaagaagctctctgctcagcctgtcacaacctactgactgccagggca

SEQ ID NO: 275 - RNA, 72 nt, synthetic construct
  source 1..72: mol_type = other RNA
cttgggaatggcaaggaaaccgttaccattactgagtttagtaatggtaatggttctcttgctatacccaga

SEQ ID NO: 276 - RNA, 72 nt, synthetic construct; identical to SEQ ID NO: 275
  source 1..72: mol_type = other RNA

SEQ ID NO: 277 - DNA, 94 bp, Homo sapiens
  source 1..94: mol_type = unassigned DNA
aaacgtgccaggaagagaactcaggaccctgaagcagactactggaagggagactccagctcaaacaaggcaggggtgggggcgtgggattggg

SEQ ID NO: 278 - DNA, 37 bp, Homo sapiens
  source 1..37: mol_type = unassigned DNA
gttccaaatgaccttccacctgtttattgctgcattt

SEQ ID NO: 279 - DNA, 93 bp, Homo sapiens; identical to SEQ ID NO: 258
  source 1..93: mol_type = unassigned DNA

SEQ ID NO: 280 - RNA, 71 nt, synthetic construct; identical to SEQ ID NO: 260
  source 1..71: mol_type = other RNA

SEQ ID NO: 281 - RNA, 71 nt, synthetic construct
  source 1..71: mol_type = other RNA
gcgacaataaacaggaggaaggtcatgctgtgaagccacagatgggcatgacctttcctgtttattgctgc

SEQ ID NO: 282 - DNA, 94 bp, Homo sapiens; identical to SEQ ID NO: 261
  source 1..94: mol_type = unassigned DNA

SEQ ID NO: 283 - RNA, 71 nt, Homo sapiens; identical to SEQ ID NO: 260
  source 1..71: mol_type = unassigned RNA

SEQ ID NO: 284 - RNA, 71 nt, synthetic construct
  source 1..71: mol_type = other RNA
gcgacaataaacaggtggaaggtcatgctgtgaagccacagatgggcatgaccttacctgtttattgctgc

SEQ ID NO: 285 - RNA, 63 nt, synthetic construct
  source 1..63: mol_type = other RNA
acctctttcttcttcctttatgtagtgaagccacagatgtacataaaggaagaagaaagaggc